(19) United States
(12) Patent Application Publication
(10) Pub. No.: US 2022/0293275 A1
(43) Pub. Date: Sep. 15, 2022

(54) METHOD FOR DETECTING GUT DYSBIOSIS OF INFANT

(71) Applicant: CJ Bioscience. Inc, Seoul (KR)

(72) Inventors: Hyeonseok OH, Seoul (KR); Uigi MIN, Seoul (KR); Namil KIM, Seoul (KR)

(21) Appl. No.: 17/617,667

(22) PCT Filed: Jun. 10, 2020
(86) PCT No.: PCT/KR2020/007494

§ 371 Date: Dec. 9, 2021

(30) Foreign Application Priority Data
Jun. 11, 2019 (KR) 10-2019-0068576

(51) Int. Cl. G16H 50/30; G16H 50/70; A61B 5/00; G06F 17/18

(52) U.S. Cl. CPC G16H 50/30; G16H 50/70; A61B 5/7275; G06F 17/18

(57) ABSTRACT

The present invention relates to a method for providing information on gut microbiota dysbiosis of a test infant by using an analysis of the gut microbiota of the infant, and can be applied for diagnosing dysbiosis in the infant or to quantitatively predicting dysbiosis in the infant. More particularly, the present invention provides a method for defiling a developmental stage of gut microbiota and a degree of gut microbiota dysbiosis in infant by using an analysis of the gut microbiota thereof, and providing information on gut microbiota dysbiosis in infant on the basis of the developmental stage.

### TECHNICAL FIELD

The present disclosure relates to a method for detecting gut microbiota dysbiosis in infants by analyzing samples and/or metadata information of infants and a method for alleviating dysbiosis in infants on the basis of results from the detection method.

### BACKGROUND ART

In the human body, more microbes than human cells live, interacting with the human body at various sites such as the skin, the digestive system, the respiratory system, etc. Most microbes present in the human body dwell in the gut. Repeated research and development of experimental techniques have gradually discovered the functions and effects of gut microbes. Gut microbes are related to human immunity and nutritional absorption, and affect even mood or mental affairs by controlling the secretion of the stress hormone cortisol. Gut microbes vary depending dietary habits and host conditions and differ in formation mode from one person to another.

Human gut microbes begin to form just after birth and play an important role in terms of immunity, metabolism, and nutrition in the early stage of life. The composition of gut flora in infants is distinctively different from that of gut flora in adults, with gradual resemblance therebetween in group structure with time of lactating foods, weaning foods, and general foods. In addition to intake of nutrition, main factors that have great influence on the formation of gut flora include delivery modes such as natural delivery or cesarean section, administration of antibiotics, and the like.

Dysbiosis is a term referring to the condition of having imbalances in the microbial communities in the body. There has recently been increasing research that reveals the direct or indirect association of dysbiosis with modern diseases such as inflammatory bowel disease, obesity, diabetes mellitus, autism, and so on. Dysbiosis is known to be caused by factors including indiscriminate intake of processed foods, antibiotic use, etc.

Research on the gut microbiomes in infants has been conducted from the perspective of the succession process and the maturity of gut microbes in infants, but still remains insufficient in terms of the interpretation of gut microbes from the view of dysbiosis. The gut microbial ecosystem in infants is not stable even when they are growing normally. It is therefore necessary to attempt to make a proper definition of dysbiosis in infants according to the maturity stage. The identification of the dysbiosis in infants using a database including gut microbial data and/or metadata is an essential research topic for interactions with the human body, such as physiological functions and immunity of gut microbes.

### DISCLOSURE

### Technical Problem

In the present disclosure, a database of gut microbiomes in infants are constructed using a non-culturing analysis method, and research has been conducted into significant patterns of gut microbiomes by considering various metadata which are an indicator accounting for dysbiosis together. In addition, infant developmental stages, infant dysbiosis states, and index species for infant dysbiosis were determined by machine learning effective for mass data analysis from the perspective of gut microbes.

An aspect of the present disclosure is to provide a method for detecting or analyzing a degree of gut microbiota dysbiosis in infants through analysis of gut microbiome in infant. The infant gut microbiome analysis may utilize a microbial biomarker detecting the infant gut dysbiosis.

An aspect of the present disclosure is to provide a method for detecting or analyzing a degree of gut microbiota dysbiosis in infants through analysis of gut microbiome in infant. The infant gut microbiome analysis may utilize microbial biomarkers detecting the infant gut dysbiosis.

An additional aspect of the present disclosure is to provide a composition or a kit for detecting gut dysbiosis degree and/or gut developmental stages in infants, comprising the microbial biomarkers detecting the infant gut dysbiosis or an agent for detecting the biomarker.

An additional aspect of the present disclosure is to provide a composition or a kit for detecting infant gut maturity by using gut microbes, the composition or kit comprising a microbial biomarker for detecting gut dysbiosis in infants or an agent for detecting the biomarker.

An additional aspect of the present disclosure pertains to a method for alleviating the infant dysbiosis obtained above or for increasing gut microbial maturity in infants. An additional aspect of the present disclosure is to provide a kit for determining dysbiosis or predicting a degree of risk of dysbiosis in infants, comprising an agent for detecting the biomarker.

### Technical Solution

The present disclosure is to provide a biomarker for determining dysbiosis in an infant or for predicting a degree of risk of dysbiosis in an infant, with accuracy at the level of genus or species in culture-independent methods (CIMs). In addition, the present disclosure pertains to a method for determining dysbiosis or predicting a degree of risk of dysbiosis in an infant. Accordingly, the present disclosure pertains to a method for improving a growth condition of an infant, the method comprising an additional treatment step for solving dysbiosis. In detail, the present disclosure may provide information on diagnosis of dysbiosis and growth state in an infant by analyzing a gut microbiome in the infant.

In the period of infancy, gut microbial environments are created and gut microbiota continues to vary, with the gradual establishment of balanced gut microbial environments. Unlike adults, the determination of the balance or imbalance of gut microbiome in infants should thus be established in consideration of various factors. Accordingly, the present disclosure is to probe an imbalance or a developmental state of gut microbiota by using gut microbiome so as to identify whether gut microflora is balanced or imbalanced in infants, or the developmental stage of gut microbiota. More preferably, the imbalance and developmental stage of microbiota may be probed from a combination of metadata information of infants, such as information on months of age, diet, delivery modes, history of antibiotic administration, and soon.

As used herein, the term “imbalanced group of gut microbiota” or “dysbiosis” in an infant refers to a sample group possessing a gut microbiome that has a positive correlation with an imbalance of gut flora or is associated with metadata contributing to or incurring an imbalance of gut flora. The term “balanced group” refers to a sample group possessing a gut microbiome that has a positive correlation with balance of gut flora or is associated with metadata contributing to or incurring balancing of gut flora.

For example, the metadata associated with an imbalance of gut flora include diarrhea, cesarean dissection, administration of antibiotics, and formula feeding, which are known to incur an imbalance of microflora. A sample group distinct from dysbiosis-associated groups within the same developmental stage is related to breast feeding and natural delivery and as such, can be defined as a group related to the balance of gut microflora. Metadata factors that strongly correlate with imbalance and balance of gut microflora are summarized as follows. The developmental stage of infants is divided into groups 1 and 2. In developmental stage 1, a balance group includes natural delivery and breast feeding while an imbalance group includes diarrhea and a history of antibiotic administration. In developmental stage 2, a balance group includes natural delivery and an imbalance group includes diarrhea and a history of antibiotic administration.

The developmental stage of infants can be determined on the basis of at least one standard selected from the group consisting of a dietary step, months of age, and an infant development index (based on information on gut microbiome). Infant development indices can be divided according to biomarkers characteristic of developmental stages, and species of microbial biomarkers characteristic of each developmental stage and a proportion (abundance ratio) of the species in gut microflora are analyzed to calculate a development index. A cut-off value is set for the development index by using accuracy, sensitivity, and specificity. Developmental stage 1 is given to a case where the development index is less than the cut-off value while developmental stage 2 is given to a case where the development index is as high as or higher than the cut-off value. According to an embodiment of the present disclosure, microbial biomarkers characteristic of infant developmental stages are exemplified by the biomarkers characteristic of infant developmental stage 1 in Tables 10 and 11, below and by the biomarkers characteristic of infant developmental stage 2 in Tables 12 and 13, below.

The term “dysbiosis” in infants refers to similarity with the gut microbiome environment (balance of gut microflora) in conformity with the growth rate of infants or with the development state of gut microbiome in infants. In the present disclosure, a degree of imbalance of gut microbiome in an infant is detected to figure out the development state of gut microbiome in the infant, and a balance of gut microbiome is achieved at a suitable rate in conformity with the growth rate of the infant.

In an exemplary embodiment of the present disclosure, the discrimination between gut microbe imbalance and balance groups in infants according to the present disclosure may be conducted using biomarkers characteristic of the imbalance group and the balance group by developmental stage.

In detail, the biomarkers characteristic of the balance group in developmental stage 1 are listed in Tables 29 and 30 and depicted in the phylogenetic tree of FIG. 10. The biomarkers characteristic of the imbalance group in developmental stage 1 are listed in Tables 33 and 34 and depicted in the phylogenetic tree of FIG. 11. In addition, biomarkers characteristic of the balance group in developmental stage 2 are listed in Tables 31 and 32 and depicted in FIG. 12 while biomarkers characteristic of the imbalance group in developmental stage 2 are listed in Tables 35 and 36 and depicted in FIG. 13.

The detecting results of dysbiosis in infants, analyzed by the probing method according to the present disclosure, can provide information on microbiota age, commensal diversity, beneficial microbes, or gut microbial dominant species in infant peers. The term “microbiota age” refers to maturity of gut microbiome in conformity with the growth rate of infants and may be expressed as, for example, a balance or imbalance of gut microbiome by months of age of infants. Commensal diversity means a diversity of gut microbial species and may be expressed by various kinds of microbes existing in the intestine. The term “beneficial microbes” means a distribution of microbes that have positive influence on the growth of infants. Insufficient development of beneficial microbes in gut microbiome increases a risk of disease. Lactobacillus contribute to nutrient absorption and strengthens immunity by helping digestion in early gut microbiome, and when the gut microbiome becomes stable, the Lactobacillus bacteria disappear and decrease in relative abundance.

Below, a method for detecting a developmental stage of gut microbiota and/or a degree of gut microbiota dysbiosis in infant will be described in detail in a stepwise manner.

In an embodiment of the present disclosure, the method for probing dysbiosis in infants may comprise the following steps of:

(A) obtaining gut microbiome information of microbial types discriminated at a species level and abundance ratios of the microbial types for gut microbiome in a test infant;

(B) obtaining metadata information of the test infant;

(C) determining a developmental stage of the gut microbiome according to criteria for classifying developmental stage of a reference infant, on the basis of at least one selected from the group consisting of the gut microbiome information of step (A) and the metadata information of step (B); and

(D) determining the degree of the gut microbiota dysbiosis according to the determined developmental stage, by using biomarkers characteristic of imbalance group and biomarkers characteristic of balance group in each developmental stage.

According to an embodiment of the present invention, in greater detail, the step (A) of obtaining gut microbiome information of microbial types discriminated at a species level and abundance ratios of the microbial types for gut microbiome in a test infant in the method for detecting a degree of gut microbiota dysbiosis in infant can be achieved in various manners and, for example, may comprise the steps of:

(A-1) obtaining genomic DNA of gut microbes from a fecal specimen of the test subject;

(A-2) obtaining 16S rRNA genetic information from the genomic DNA of the gut microbes; and

(A-3) analyzing the gut microbiome information of microbial types discriminated at a species level and abundance ratios of the microbial types for gut microbiome in the test subject, by performing an analysis of the 16S rRNA information of gut microbes.

(A-1) Step of Obtaining Genomic DNA of Gut Microbes from a Fecal Specimen of the Test Subject

The subject to be tested may be an infant. As used herein, the term “infant” refers to a newborn or baby at 36 months or less of age.

In an embodiment of the present disclosure, a total of 120 fecal samples were collected from infants at 4 weeks to 3 years (36 months) of age through respective legal guardians thereof according to the stipulations of the World Health Organization (WHO). The fecal samples of the subjects to be tested were collected in a buffer preventive of the mutation of microbes. The buffer contained 4% (w/v) SDS (Sodium Dodecyl Sulfate), 50 mM Tris-HCl, 50 mM EDTA, and 500 mM NaCl.

The step of obtaining DNA from the collected fecal samples may be conducted in culture-independent methods (CIMs). Use of the culture-independent methods can prevent data distortion that may be generated during the cultivation of microbes, and allows the acquisition of information on a microbiome composition similar to an actual gut microbial ecosystem.

(A-2) Step of Obtaining 16S rRNA Genetic Information from the Genomic DNA of the Gut Microbes

The step of obtaining 16S rRNA genetic information may be a step of sequencing 16S rRNA genes of the extracted DNA through a next-generation sequencing (NGS) platform.

The step of sequencing 16S rRNA genes of the extracted DNA may comprise a step of performing PCR with a set of primers capable of specifically amplifying a variable region of 16S rRNA, preferably with a set of primers capable of specifically amplifying V3 to V4 regions of 16S rRNA, and more preferably with universal primers having the following sequences, to produce an amplicon. Exemplary sequences of the universal primers are as follows:

Forward universal primer (SEQ ID NO: 161):
5′-CCTACGGGNGGCWGCAG-3′
Reverse universal primer (SEQ ID NO: 162):
5′-GACTACHVGGGTATCTAATCC-3′

(A-3) Step of Analyzing the Gut Microbiome Information of Microbial Types Discriminated at a Species Level and Abundance Ratios of the Microbial Types for Gut Microbiome in the Test Infant, by Performing an Analysis of the 16S rRNA Information of Gut Microbes

The analysis of gut microbiome information may be conducted by a step of analyzing bacterial community information at levels from phylum to species with the aid of the 16S ribosomal RNA gene sequence database (EzTaxon) of standard strains and non-cultured microbes and the EasyBioCloud analysis system (http://www.ezbiocloud.com) on the basis of thousands of gene sequences generated by the next-generation sequencing technique from one sample. When products of the next-generation sequencing technique are identical, the method for microbiome information analysis is not limited to Eztaxon and the EasyBioCloud analysis system.

The microbial biomarker may be selected based on a ratio of the number of times each microbe is determined to be characteristic of a specific developmental stage to the total number of bootstrap repetitions for performing the machine learning. Preferably, after the selecting step of the microbial marker, when the microbiome composition for the selected biomarker in the corresponding developmental stage is lower than that in other developmental stages, a verification step exclusive of the corresponding microbial marker, may be further included. Preferably, the microbial marker may be a microbe possessing 16S rRNA including at least one of the nucleotide sequences of SEQ ID NOS: 1 to 160.

The step of analyzing microbiome may comprise a step of analyzing a composition of the microbial biomarkers possessing at least one of the nucleotide sequences of SEQ ID NOS: 1 to 160 by using a database of 16S rRNA sequences of standard strains and non-cultured microbes. The step of analyzing microbiome is designed to determine the presence or absence of microbes possessing 16S rRNA sequence selected from the sequences of SEQ ID NOS: 1 to 160 provided in the present disclosure and analyze only the microbes identified to be present, whereby time and labor necessary for dysbiosis determination and prognosis prediction in infants can be reduced, compared to identifying a composition of entire microbes.

The step of analyzing microbiome may comprise a step of identifying and classifying microbes at a level of genus or species and/or a step of analyzing a composition of each microbiome.

The database used for identifying and classifying microbes may be appropriately selected, as necessary, by a person skilled in the art. For example, the database may be at least one selected from the group consisting of EzBioCloud, SILVA, RDP, and Greengene, with no limitations thereto.

The composition of microbiome may be expressed as a relative abundance (%) of a specific microbiome in the entire gut microflora. The relative abundance (%) of a microbiome may be a percentage of 16S rRNA read frequencies of the specific microbe in the total sequencing reads. The specific microbe may be a microbial biomarker for determining or predicting the dysbiosis in an infant provided by the present disclosure.

(B) Step of Obtaining Metadata Information of Test Infant

The method for providing information on determination or prediction of dysbiosis in infants according to the present disclosure may comprise a step (B) of collecting metadata information of test infant.

The collection of metadata may be conducted at the same time and/or a different time for the step (A-1) of collecting a fecal sample from a test infant.

So long as it is useful for determining an infant's developmental stage, health state, and/or dysbiosis state, any factor may be collected within the metadata and used for analysis. For example, data including at least one factor selected from the group consisting of an infant's sex, months of age, height, weight, diet type, feeding mode for the infant, feeding of lactic acid bacterium-containing diet, fecal type, fecal color, information on antibiotic use, information on diagnosed diseases, mother's diet type during a gestation period, and mother's diet type and antibiotic administration after delivery may be collected, but with no limitations thereto.

Among the information collected for the metadata of the present disclosure, the information on diet type may be at least one selected from the group consisting of information on ingestion of a lactic acid bacterium-containing diet, information on ingestion of fermented foods, and information on ingestion of non-fermented health functional foods or non-fermented foods, but with no limitations thereto.

In an embodiment of the present disclosure, metadata related to information on dysbiosis was collected using various questionnaire items. The step of obtaining metadata may be a step in which answers to questionnaire items including factors that have influence on dysbiosis are appended to the analyzed 16S rRNA sequence data and stored in a database.

Specific questionnaire items were divided into three dietary types: A (feeding), B (weaning), and C (general) so that the questionnaire could be filled out with corresponding dietary types at the time of sample collection, and questionnaire types were selected by the judgment of legal guardians of the test infants. The questionnaire items consist of delivery modes, breastfeeding methods, types of weaning foods, baby foods, and general foods, and types of feces. Specific questionnaire items are given in Table 2.

(C) Step of Determining a Developmental Stage of the Gut Microbiome According to Criteria for Classifying Developmental Stage of a Reference Infant, on the Basis of at Least One Selected from the Group Consisting of the Gut Microbiome Information of Step (A) and the Metadata Information of Step (B)

In the step of selecting classification criteria for a developmental stage of a test infant, the criteria may include at least one selected from the group consisting of the dietary stage, months of age, and infant development index (based on information on gut microbiome). With respect to the infant development index, the biomarkers in Tables 10 to 13 below are biomarkers that classify the developmental stage of gut microbiome of infants, and use the final biomarkers secondarily selected. In Table 14 below, methods for determining developmental stages according to determination criteria for the developmental stages are summarized.

In detail, developmental stages of infants may be discriminated using dietary steps, months of age, or biomarkers characteristic of developmental stages.

The method for determining developmental stages of infants by using biomarkers characteristic of developmental stages comprises a step of applying analysis results of 16S rRNA collected from feces of the test infants to a gut microbial developmental stage prediction model of infants to calculate infant development indices.

The criteria for classifying developmental stage of a reference infant are obtained by performing the steps comprising:

(A′) obtaining gut microbiome information of microbial types discriminated at a species level and abundance ratios of the microbial types for gut microbiome in the reference infant;

(B′) obtaining metadata information of the reference infant; and

(C′) determining criteria for classifying developmental stage of the reference infant, on the basis of at least one selected from the group consisting of the gut microbiome information of step (A′) and the metadata information of step (B′).

The step (C′) of determining classification criteria for developmental stages of the reference infant may be conducted using at least one selected from the group consisting of dietary step, months of age, and microbial biomarkers characteristic of developmental stages.

In the step (C) of determining developmental stage, the classification criteria for developmental stages of a reference include solid diet feeding, whether the infant is older than 15 months or not, and whether the infant development index meets 1.19 or not.

Classification of Infant Developmental Stage in Terms of Dietary Step

The classification of infant developmental stages through dietary steps is designed to divide diets of infants into liquid-type feeding foods, gel-type weaning foods, solid-type infant foods, and solid-type general foods, and to set the dietary step of liquid-type feeding foods or gel-type weaning foods as developmental stage 1 and the dietary step of solid-type foods, that is, infant foods or general foods as developmental stage 2 on the basis of the metadata information (diet) of infants. Thus, the time at which infants fed with liquid- or gel-type feeding foods or weaning foods ingest a solid-type food is a criterion for infant developmental stages.

Classification of Infant Developmental Stage in Terms of Months of Age

For classification of developmental stages according to months after birth (months of age), developmental stage 1 is set for a test infant who is under 15 months after birth and developmental stage 2 is set when a test infant is 15 or more months old. The criterion of 15 months was defined with reference to the time when the diet type is converted from gel-type to solid-type foods and the time when the data groups were classified through the DMM grouping method of Example 4-2. Therefore, the criterion of 15 months defined by the above method means the time when the dietary steps are most clearly divided and when microbial compositions and their respective abundance ratio in gut microbiome are most greatly changed. Infant gut microbes consist mainly of microbes that contribute to immunity, digestion of breast milk, and intestinal stabilization, immediately after birth, and exhibit a greatly increased spectrum of microbial kinds with the predominance of microbes associated with metabolisms of various foods, such as dietary fibers, etc., since the time of 15 months after birth.

Classification of Infant Developmental Stage in Terms of Biomarker Characteristic of Developmental Stage

In a case where an infant development index is adopted as a criterion, kinds (species) of each microbial biomarker characteristic of developmental stage and a proportion (abundance ratio) of the characteristic species in gut microflora are analyzed on the basis of the microbiome analysis data for the collected gut microbes and applied to the above-mentioned prediction model for infant developmental stage to classify the developmental stage.

In case where developmental stage 1 and developmental stage 2 may be classified by using microbial biomarkers characteristic of developmental stages, the biomarker characteristic of developmental stage 1 may be at least one selected from the group consisting of microbes listed in Tables 10 and 11 while the biomarker characteristic of developmental stage 2 may be at least one selected from the group consisting of microbes listed in Tables 12 and 13.

Species of microbial biomarkers characteristic of each developmental stage and a proportion (abundance ratio) of the species in gut microflora are analyzed to calculate a development index, and a cut-off value is set for the development index in terms of accuracy, sensitivity, and specificity. Developmental stage 1 is given to a case where the development index is lower than the cut-off value, while developmental stage 2 is given to a case where the development index is equal to or higher than the cut-off value.

In the step (C′) of determining classification criteria for developmental stages of the reference infant, species of microbial biomarkers characteristic of developmental stages 1 and 2 and a proportion (abundance ratio) of the species in gut microflora are analyzed to calculate a development index, and a cut-off value is set for the development index in terms of accuracy, sensitivity, and specificity. Developmental stage 1 is given to a case where the development index is lower than the cut-off value, while developmental stage 2 is given to a case where the development index is equal to or higher than the cut-off value.

In the step (C) of determining developmental stage, species of microbial biomarkers characteristic of developmental stages of test infants and proportions (relative abundance) of the species in gut microflora are analyzed to calculate development indices of the infants, and developmental stage 1 is given to a case where the development index is lower than the cut-off value which is the classification criterion for developmental stages of the reference infant, while developmental stage 2 is given to a case where the development index is equal to or higher than the cut-off value.

In the present disclosure, as explained in Examples 4-7, decision is made of developmental stage 1 for a measurement being lower than the development index 1.19 and developmental stage 2 for a measurement being equal to or higher than the development index 1.19.

When developmental stages are classified according to answers to a questionnaire for dietary steps and months after birth, answers to the questionnaire including items of Table 2 should take precedence. For classification on the basis of the infant development index, analysis of gut microbes using the method of Example 2 should take precedence.

The gut microbial ecosystem of infants is established as microbes residing in parents and surrounding environments are transferred to and settled down in newborns free of germs, and the abundance and diversity of microbial species in infants increase with their growth and diet. In this increasing trend, biomarkers characteristic of developmental stages of infants account specifically for the development pattern of the intestinal microbial ecosystem according to the growth of infants. Biomarkers characteristic of developmental stage 1 are given in Tables 10 and 11, while biomarkers characteristic of developmental stage 2 are listed in Tables 12 and 13.

The infant development index may be calculated using the following Mathematical Formulas 4 to 7:

$$\begin{matrix}p = \frac{1}{1 + {e}^{- β \cdot X}} = {logit}^{- 1} ( β \cdot X ) = {logit}^{- 1} ( {β}_{0} + {\sum }_{j = 1}^{m} {β}_{j} {x}_{j} ) & [ Mathematical Formula 4 ] \\ {min}_{β} λ {‖ β ‖}_{1} + {\sum }_{i = 1}^{n} log ( {e}^{- {y}_{i} ( β \cdot {X}_{i} )} + 1 ) & [ Mathematical Formula 5 ] \\ \hat{p} = {logit}^{- 1} ( \hat{β} \cdot {X}^{′} ) = \frac{1}{1 + {e}^{- \hat{β} \cdot {X}^{′}}} & [ Mathematical Formula 6 ] \\ Infant development index = \frac{\hat{p}}{{p}_{o}} = \frac{\hat{p}}{{N}_{case} / {N}_{train}} & [ Mathematical Formula 7 ]\end{matrix}$$

(D) Step of Determining Whether the Gut Microbiome is an Imbalance Group or a Balance Group According to the Determined Developmental Stage by Referring to Biomarkers Characteristic of Imbalance Groups by Developmental Stage and Biomarkers Characteristic of Balance Groups by Developmental Stage.

Determination of dysbiosis in a test infant according to the selected developmental stages of the infant may be conducted by utilizing biomarkers characteristic of imbalance groups by developmental stage and biomarkers characteristic of balance groups by developmental stage.

Discrimination between the gut microbiome imbalance and balance groups of infants according to the present disclosure may be performed using a biomarker characteristic of the imbalance group by development stage and a biomarker characteristic of the balance group by development stage.

In detail, biomarkers characteristic of the balance group of developmental stage 1 are listed in Tables 29 and 30 and depicted in the phylogenetic tree of FIG. 10 and biomarkers characteristic of the imbalance group of developmental stage 1 are listed in Tables 33 and 34 and depicted in the phylogenetic tree of FIG. 11. In addition, biomarkers characteristic of the balance group of developmental stage 2 are listed in Tables 31 and 32 and depicted in the phylogenetic tree of FIG. 12 and biomarkers characteristic of the imbalance group of developmental stage 2 are listed in Tables 35 and 36 and depicted in the phylogenetic tree of FIG. 13.

In a particular embodiment, the determining step comprises the step of applying the analysis result of 16S rRNA collected from feces of the test infants to an infant dysbiosis prediction model to calculate an infant dysbiosis index.

The infant dysbiosis prediction model is to provide a parameter for calculating a dysbiosis index of a test infant by comparing a gut microbiome composition of microbial biomarkers for predicting infant gut microbe imbalance and/or balance with a database.

The infant dysbiosis prediction model is utilized for determining and/or predicting infant dysbiosis by applying a list of biomarkers characteristic of infant imbalance and/or balance groups, detected in the test infants, and coefficient values to a machine learning function and indexing mathematical formulas (mathematical formulas 1 to 7) to calculate a dysbiosis index for an unknown sample.

The database may utilize a database of gut microflora in an infant sample group collected to specify a microbial biomarker and, specifically, may be a human gut microbiome database recruited from infants aged more than 4 weeks to under 3 years (36 months).

The infant dysbiosis prediction model is characterized by an ability to select microbial biomarkers characteristic of gut microbe imbalance and/or balance groups through machine learning and to determine infant dysbiosis by calculating the infant dysbiosis index.

The step of indexing the microbiome analysis result may comprise applying the result to the machine learning functions and indexing mathematical formulas (Mathematical Formulas 1 to 7) and calculating an infant dysbiosis index for infant dysbiosis determination by using microbial biomarkers and coefficient values of the corresponding markers.

After the step (C′) of determining classification criteria for developmental stages of the reference infant, the method may further comprise a step (D′) of selecting determination criteria for imbalance groups by developmental stage. In step (D′), the determination criteria for imbalance groups by developmental stage may be used for determining species of microbial biomarkers characteristic of balance and imbalance groups of each developmental stage in the reference infant, analyzing a proportion (relative abundance) of the species in gut microflora to calculate a development index, setting a cut-off value for the development index in terms of accuracy, sensitivity, and specificity, and determining a balance group for a development index measurement less than the cut-off value and an imbalance group for a development index measurement as high as or higher than the cut-off value.

The step of determining whether the infant to be tested is in a dysbiosis state may comprise a step of determining a position of the index on the distribution of infant dysbiosis indices in the entire database. When the infant dysbiosis index is included within or is closer to the balance section in the infant dysbiosis index distribution of the entire database, the prognosis of dysbiosis may be determined to be improved. The entire database may be, for example, an infant dysbiosis index database of all samples including the training set, the test set, and the test sample used in the construction of the prediction model, but is not limited thereto.

The step (D) of determining whether the gut microbiome is an imbalance group or a balance group may be conducted by determining species of microbial biomarkers characteristic of balance and imbalance groups of each developmental stage in the reference infant, analyzing a proportion (abundance ratio) of the species in gut microflora to calculate an imbalance determination index, and determining the test infant as a balance group when the calculated imbalance determination index of the test infant is less than a cut-off value set as a reference criterion for the imbalance determination index of a reference infant and as an imbalance group when the calculated imbalance determination of the test infant is as high as or higher than the cut-off value.

The imbalance determination index can be calculated using the following Mathematical Formulas 4 to 7:

$$\begin{matrix}p = \frac{1}{1 + {e}^{- β \cdot X}} = {logit}^{- 1} ( β \cdot X ) = {logit}^{- 1} ( {β}_{0} + {\sum }_{j = 1}^{m} {β}_{j} {x}_{j} ) & [ Mathematical Formula 4 ] \\ {min}_{β} λ {‖ β ‖}_{1} + {\sum }_{i = 1}^{n} log ( {e}^{- {y}_{i} ( β \cdot {X}_{i} )} + 1 ) & [ Mathematical Formula 5 ] \\ \hat{p} = {logit}^{- 1} ( \hat{β} \cdot {X}^{′} ) = \frac{1}{1 + {e}^{- \hat{β} \cdot {X}^{′}}} & [ Mathematical Formula 6 ] \\ Infant dysbiosis index = \frac{\hat{p}}{{p}_{o}} = \frac{\hat{p}}{{N}_{case} / {N}_{train}} & [ Mathematical Formula 7 ]\end{matrix}$$

The infant dysbiosis index is expressed for at least two sections into which the distribution interval of infant dysbiosis index is divided and preferably for the three discrete sections of propriety, fastness, and slowness according to developmental stage.

The interval may be divided based on the highest value for specificity for the dysbiosis index of infants.

According to an embodiment of the present disclosure, imbalance and balance steps in each of developmental stage 1 and developmental stage 2 are classified with reference to the dysbiosis index. Classification is made of a balance step for a dysbiosis index corresponding to a lower limit of 0% to 70% and an imbalance step for a dysbiosis index corresponding to a lower limit of 70% to 100% in each developmental stage.

In greater detail, a propriety step is classified for a dysbiosis index corresponding to a lower limit of 0% to 70% and a “fastness” step for a dysbiosis index corresponding to a lower limit of 70% to 100% in developmental stage 1 and a propriety step is classified for a dysbiosis index corresponding to a lower limit of 0% to 70% and a “slowness” step for a dysbiosis corresponding to a lower limit of 70% to 100% in developmental stage 2.

In the classification, the “fastness” and “slowness” steps are defined on the basis of the feature where the biomarkers characteristic of dysbiosis in developmental stage 1 are microbes dominant in developmental stage 2 and biomarkers characteristic of dysbiosis in developmental stage 2 are microbes dominant in developmental stage 1.

The method for providing information on prediction of dysbiosis in an infant may further comprise a step of monitoring a change of dysbiosis index in the test infant with time.

The step of monitoring a change of dysbiosis index in the infant with time may be characterized in that the prognosis is determined to become better over time as the distribution section of the infant dysbiosis index approaches the lower limit of 0%.

The method for detecting dysbiosis of an infant may further comprise a step (E) of alleviating dysbiosis or improving gut maturity in the infant by conducing at least one selected from the group consisting of suggestions for prebiotics, probiotics, medication, diets, and life habits according to the group determined in the step of determining whether the gut microbiome of the infant is balanced or not.

For the probiotics or prebiotics, the type and content of microbes may be determined using the gut microbe developmental stage and the dysbiosis in the test infant.

Examples of the probiotics may include marker microbes for the gut microbial balance group in the reference infant group according to the developmental stage of the infant gut microbes. In detail, the probiotics include at least one microbial biomarker characteristic of the balance group of developmental stage 1, shown in Tables 29 and 30, when the test infant is analyzed to be in developmental stage 1 and at one microbial biomarker characteristic of the balance group of developmental stage 2 when the test infant is analyzed to be in developmental stage 2.

In addition, when the test infant is analyzed to be under developmental stage 1, the prebiotics may include a material inducing an increase in the relative abundance of at least one of the microbial biomarkers characteristic of the balance group of developmental stage 1, listed in Tables 29 and 30, and/or a material inducing a decrease in the relative abundance of at least one of the microbial biomarkers characteristic of the imbalance group of developmental stage 1, listed in Tables 33 and 34. Alternatively, when the test infant is analyzed to be under developmental stage 2, the prebiotics may include a material inducing an increase in the relative abundance (relative abundance) of at least one of the microbial biomarkers characteristic of the balance group of developmental stage 2, listed in Tables 31 and 32, and/or a material inducing a decrease in the relative abundance of at least one of the microbial biomarkers characteristic of the imbalance group of developmental stage 2, listed in Tables 35 and 36.

The provision of infant dysbiosis index through biomarkers characteristic of the infant gut microbial imbalance group and/or balance group and the infant dysbiosis prediction model using the same may be conducted through the following steps of:

(1) collecting a fecal sample from a test infant,

(2) extracting DNA of a target subject from the fecal sample and performing PCR in the presence of universal primers for 16S rRNA, with the extracted DNA serving as a template, to generate amplicons,

(3) sequencing 16S rRNA genes of the amplicons through a next-generation sequencing (NGS) platform,

(4) analyzing the 16S rRNA gene sequences by using a database of 16S rRNA gene sequences of standard strains and non-culture microbes to perform a microbiome analysis of the target subject,

(5) collecting metadata including dysbiosis-related items from the test infant,

(6) determining a developmental stage of the test infant, on the basis of the result of (4) or (5) according to the classification of developmental stages for a reference infant,

(7) comparing microbiomes of gut microbial imbalance and balance groups and relative abundances of constituent microbes between the test infant and relative abundance of a reference infant microbial composition which is in the corresponding developmental stage of the test infant, and

(8) determining an imbalance of the gut microbiome in the test infant when the infant meets an infant dysbiosis index criterium as a result of the comparison.

The infant dysbiosis prediction results may be indexed and provided as an analysis report. The analysis report may include the following information.

(1) Developmental stage and dysbiosis index of test subject

The report includes the results of calculating infant dysbiosis indices by applying an infant dysbiosis prediction model to the infants.

(2) Information on dysbiosis microbial biomarkers detected in infants

In addition, the analysis report may indicate the description and ratio of representative microbes among microbes corresponding to the dysbiosis biomarkers of infants.

### Advantageous Effects

The infant dysbiosis biomarker provided by the present disclosure allows the decision of infant dysbiosis with respect to gut microbe analysis data. Specifically, the present disclosure provides an infant dysbiosis biomarker, and a method or a kit for determining or predicting infant dysbiosis, using same, whereby it is possible to determine infant dysbiosis or to quantitatively predict infant dysbiosis.

### DESCRIPTION OF DRAWINGS

FIG. 1 is a schematic diagram illustrating sample pretreatment and quality control steps for analyzing infant gut microbiome according to an embodiment of the present disclosure.

FIG. 2 is a map showing changes in relative abundance of 11 taxonomic groups of gut microbes with age (month).

FIGS. 3A and 3B are plots of dietary step distributions against months of age of infants. FIG. 3A show distributions of lactating food, weaning food, and general food steps and FIG. 3B shows distributions of early, middle, and late stages of weaning food and baby food step.

FIGS. 4A and 4B are plots of infant samples grouped using the DMM grouping method of Example 3-1 according to developmental stage. In FIG. 4A, circular dots indicate the first group while triangular dots indicate the second group. FIG. 4B shows distributions of cluster 1 and cluster 2 against months of age of infants wherein the horizontal axis and the vertical axis mean months of age and distribution density of sample, respectively, and the vertical lines stand for reference months of age at which the two groups cross.

FIG. 5 is an ROC and AUC plot showing results as assayed for developmental stages of the test set by a machine learning model for determining infant developmental stages assayed.

FIG. 6 is a plot showing sensitivity, specificity, and accuracy results calculated according to cut-off values so as to select determination indices for infant developmental stages according to Example 4-7, in which the horizontal axis means cut-off values and the vertical axis means sensitivity, specificity, and accuracy values calculated.

FIGS. 7A and 7B are diagrams showing center coordinates of dysbiosis-related factors as calculated on the basis of the subdivision of each of infant developmental stages 1 and 2 into two groups resulting from the DMM grouping. FIG. 7A is a diagram for samples in developmental stage 1 while FIG. 7B is a diagram for samples in developmental stage 2.

FIGS. 8A and 8B are ROC and AUC plots for verifying infant dysbiosis prediction models according to each of infant developmental stages 1 and 2. FIG. 8A is a plot for samples in developmental stage 1 while FIG. 8B is a plot for samples in developmental stage 2.

FIGS. 9A and 9B are plots showing sensitivity, specificity, and accuracy results calculated according to cut-off values so as to determine whether there is a microbial imbalance in each developmental stage of infants. FIG. 9A is a plot for samples in developmental stage 1 while FIG. 9B is a plot for samples in developmental stage 2.

FIG. 10 shows phylogenetic trees of subgroups of biomarkers characteristic of the gut microbe balance group in developmental stage 1 of infants as identified at genus or species levels according to genetic distances.

FIG. 11 shows phylogenetic trees of subgroups of biomarkers characteristic of the gut microbe imbalance group in developmental stage 1 of infants as identified at genus or species levels according to genetic distances.

FIG. 12 shows phylogenetic trees of subgroups of biomarkers characteristic of the gut microbe balance group in developmental stage 2 of infants as identified at genus or species levels according to genetic distances.

FIG. 13 shows phylogenetic trees of subgroups of biomarkers characteristic of the gut microbe balance group in developmental stage 2 of infants as identified at genus or species levels according to genetic distances.

### MODE FOR INVENTION

Below, a better understanding of the present disclosure may be obtained via the following examples which are set forth to illustrate, but are not to be construed as limiting, the present disclosure.

### Example 1. Collection of Infant Sample and Metadata

1-1. Selection Criterion of Infants and Sample Collection

For this experiment, infants at 4 weeks to 3 years (36 months) of age were selected according to the regulations of the World Health Organization (WHO) and a total of 120 fecal samples was transferred from legal guardians of the infants. The feces samples were delivered while being stored in a buffer preventive of the degradation of microbes. The composition of the buffer is given in Table 1.

TABLE 1

| | Final | |
|---|---|---|
| Component | Concentration | Product name |
| SDS | 4% | 10% sodium dodecyl sulfate |
| | | (Sigma, Cat. No. 71736-500 ML) |
| Tris-HCl | 50 mM | 1M Tris-HCl, pH 8.0 |
| | | (BIOSESANG, Cat. No. T2016-8.0) |
| EDTA | 50 mM | 500 mM Ethylenediamine tetraacetic acid, |
| | | pH 8.0 (BIOSESANG, Cat. No. E2002) |
| NaCl | 500 mM | 5M Sodium chloride |
| | | (Sigma, Cat. No. 71386-1L) |

1-2. Collection of Infant Metadata

Together with collection of each sample, a questionnaire including items to figure out infant dietary habits was prepared and submitted.

The questionnaire was divided into three dietary types: A (feeding), B (weaning), and C (general) so that the questionnaire could be filled out with corresponding dietary types at the time of sample collection, and questionnaire types were selected by the judgment of legal guardians of the test infants. The questionnaire items consist of delivery modes, breastfeeding methods, types of weaning foods, baby foods, and general foods, and types of feces. Specific questionnaire items are given in Table 2.

### Example 2. Analysis of Gut Microbiome by Next Generation Sequencing (NGS)

2-1. Acquisition and Analysis of 16S Ribosomal RNA Gene Sequence

From the fecal samples collected using the method of Example 1-1, genomic DNA was extracted. All of the samples were collected while being stored in a DNA buffer. Just after collection, the samples were homogenized with FastPrep (MP Biomedicals) for 40 seconds at a speed of 6.0 to extract genomic DNA in a physical manner.

In brief, PCR was performed on the extracted genomic DNA using universal primers to various types of amplicons for a broad range of taxonomic groups. PCR pre-mix and conditions are given in Tables 3 and 4 and sequences of the universal primers are as follows.

Forward universal primer (SEQ ID NO: 161):
5′-CCTACGGGNGGCWGCAG-3′
Reverse universal primer (SEQ ID NO: 162):
5′-GACTACHVGGGTATCTAATCC-3′

TABLE 3

| Component | Content (IX) |
|---|---|
| Template (genomic DNA) | 0.5 ul |
| 2× buffer | 10 ul |
| Forward universal primer (10 pmole) | 0.5 ul |
| Reverse universal primer (10 pmole) | 0.5 ul |
| Polymerase | 0.3 ul |
| 3′ D.W | 8.2 ul |
| Total | 20 ul |

TABLE 4

| Cycle step | Temperature | Time |
|---|---|---|
| Initial denaturation | 95° C. | 3 min |
| Denaturation annealing& | 95° C. | 30 sec |
| Extension (25 cycles) | 55° C. | 30 sec |
| | 72° C. | 30 sec |
| Final extension | 72° C. 4° C. | 5 min ∞ |

The amplicons thus produced were purified and then subjected to quality control (QC) using Bioanalyzer (Agilent), qPCR, etc. to identify the presence of 16S rRNA sequences of gut microbes therein. Thereafter, 16s ribosomal RNA gene sequences in the samples were analyzed by next generation sequence (NGS) using the MiSeq (Illumina) system.

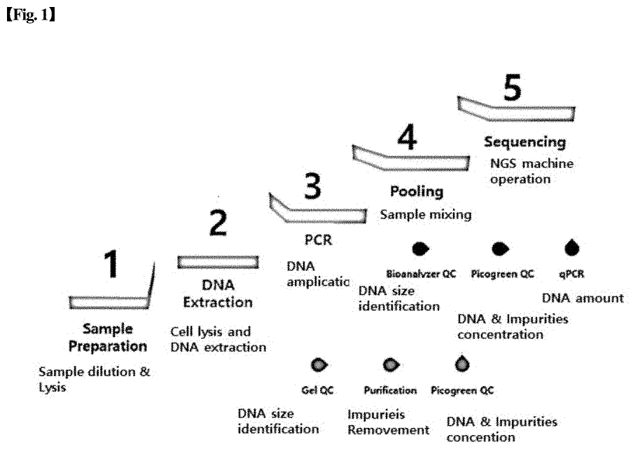

A schematic diagram illustrating sample pretreatment and QC procedures is depicted in FIG. 1. In brief, a DNA band was detected at around 650 bp in the DNA amplification process, as measured by Gel QC. The DNA sample was concentrated to 5 ng/μl as quantitatively analyzed for DNA using PicoGreen reagent. In the sample mixing step, Bioanalyser QC was performed to examine whether short peaks other than main peaks appeared in the DNA peaks. Quality control was conducted to set a DNA concentration of 5 ng/μl as measured by PicoGreen QC assay.

2-2. Microbiome Assay

After production of thousands of gene sequences from one sample by the next-generation sequencing technique, bacterial community information was analyzed at levels from phylum to species with the aid of the 16S ribosomal RNA gene sequence database (EzTaxon) of standard strains and non-cultured microbes and the EasyBioCloud analysis system (http://www.ezbiocloud.com).

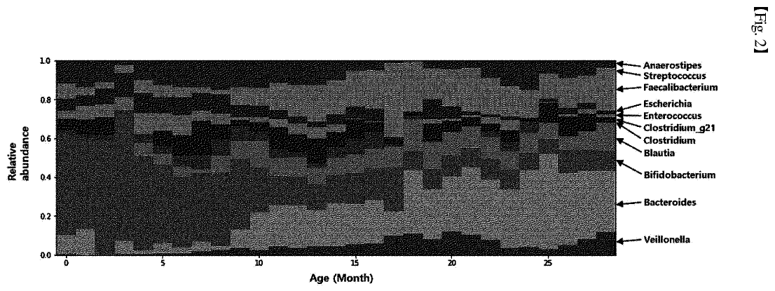

Eleven taxonomic groups of microbes which exhibited highest relative abundances in terms of 16S rRNA in the samples are depicted for relative abundance with age (month) in FIG. 2. The 11 taxonomic groups included the genus Anaerostipes, the genus Bacterioides, the genus Bifidobacterium, the genus Blautia, the genus Clostridium, an unreported genus in the family Lachnospiraceae, the genus Enterococcus, the genus Escherichia, the genus Faecalibacterium, the genus Streptococcus, and the genus Veillonella.

For details of change of the 11 taxonomic groups with months of age in infants, a decrease in the abundance of the genus Bifidobacterium and an increase in the abundance of the genera Bacteroides and Faecalibacterium appeared remarkable with the growth of infants. Bacteria in the Bifidobacterium genus, which are representative of lactic acid bacteria effective for immunopotentiation and nutritional absorption in infants, are known to be delivered to the infant gut through breast milk and help the settlement of gut microflora in the early stage. As shown in FIG. 2, when the total size of gut microflora was set to 1, the size of Bifidobacterium bacteria increased to a level of 0.7 in the second month and then decreased to a level of 0.2 after 10 months.

Microbes in the Bacteroides and Faecalibacterium genera, which show remarkable growth over time, are associated with metabolism of vegetable carbohydrates and the production of short-chain fatty acids. As the age of infant increases and the number of infants eating weaning food and baby food increases, it can be estimated that dietary fiber decomposition and short-chain fatty acid producing bacteria increase. Short-chain fatty acids, which are the main metabolites produced through degradation of dietary fiber, are known to have beneficial effects on the human body, such as promotion of energy production and vitamin production, and reinforcement of colonocyte association.

In infants before 8 months of age, the proportion of Bacteroides in the total microflora was at a very low level of about 0.05, but after 9 months, the proportion gradually increased, amounting to a level of 0.48 at two years of age.

As for microbes in the Faecalibacteium genus, their distribution appeared very low until three months of age, but the abundance ratio gradually increased after three months of age, reaching a level of 0.2 by 12 months of age and afterward was maintained at a level of about 0.25.

### Example 3. Gut Microbiome Analysis Data Grouping and Selection of Feature by Group

3-1. Gut Microbiome Analysis Data Grouping by DMM Grouping

Dirichlet multinomial mixtures (DMM) grouping is an analysis method for community grouping of microbial community profiling data including various factors and is suitable for reflecting vast amounts of gut microbiome analysis data. According to the DMM grouping method, the optimal group was found by setting the probability distribution of the gut microbiome as shown in Mathematical Formula 1, below.

$$P ( {\overline{p}}_{i} \vert Q ) = {\sum }_{k = 1}^{K} Dir ( {\overline{p}}_{i} \vert {\overline{α}}_{k} ) {π}_{k} , \\ Q = ( K , {\overline{α}}_{I} , … , {\overline{α}}_{K} , {π}_{I} , … , {π}_{K} ) \qquad [ Mathematical Formula 1 ]$$

First, the group of each sample is expressed as a probability vector p̅ᵢ (i=1, . . . , N) for the taxonomic group, wherein N accounts for the total number of samples. The probability vector is generated from the mixture prior distributions having different hyperparameters x̅ₖ (k=1, . . . , K) according to community groups in the Dirichlet distribution. K is the total number of community groups and πₖ accounts for a weight value of the mixture model.

Observed values X for samples are generated by multinomial sampling from the probability vector by group. Finally, the likelihood of an observed sample is defined as Mathematical Formula 2.

$$L ( X \vert {\overline{p}}_{1} , … , {\overline{p}}_{N} ) = {\prod }_{i = 1}^{N} {L}_{i} ( {\overline{X}}_{i} \vert {\overline{p}}_{i} ) . \\ {L}_{i} ( {\overline{X}}_{i} \vert {\overline{p}}_{1} ) = {J}_{i} ! {\prod }_{j = 1}^{S} \frac{{p}_{ij}^{{x}_{ij}}}{{X}_{ij} !} , {J}_{i} = {\sum }_{j = 1}^{S} {X}_{ij} \qquad [ Mathematical Formula 2 ]$$

Combining the likelihood and the prior distribution can obtain the posterior distribution of Mathematical Formula 3.

$$P ( {\overline{p}}_{i} \vert {\overline{X}}_{i} , Q ) = \frac{{\sum }_{k = 1}^{K} {L}_{i} ( {\overline{X}}_{i} \vert {\overline{p}}_{i} ) Dir ( {\overline{p}}_{i} \vert {\overline{α}}_{k} ) {π}_{k}}{{\sum }_{k = 1}^{K} P ( {\overline{X}}_{i} \vert {\overline{α}}_{k} ) {π}_{k}} . \qquad [ Mathematical Formula 3 ]$$

In a Bayesian approach, a hyperparameter is found for the model with the distribution maximized therein. In this regard, the Expectation-maximization algorithm was used. Model fit was determined using the Laplace approximation. This was calculated using the Dirichlet Multinomial package of the statistical analysis program R.

3-2 Machine Learning Model Construction

For machine learning, all infant samples were divided into a training set and a test set. The training set was used for training a machine learning model while the test set was used for evaluating the machine learning model. Samples from each group divided by the method of Example 3-1 were randomly selected at a ratio of about 2:1 to define a test set and a training set. When constructing the machine learning model, the sample selection process was repeated 100 times with bootstrap replication to derive the expected value of the regression coefficient, and the test set and training set were randomly reset for each bootstrap replication.

The machine learning is a step of recognizing gut microbial patterns for each of the groups divided by the method of Example 3-1, with statistical significance. The prediction model utilized least absolute shrinkage and selection operator (LASSO). LASSO's feature selection algorithm is characterized by selecting only microbes that exhibit the strongest correlation with the prediction variable that allows the division of groups by applying a penalty to a sum of regression coefficients in the model (Friedman, Hastie & Thirani, J Stat Softw, 2010., S. J. Kim, K. Koh, M. Lustig, S. Boyd and D. Gorinevsky, in IEEE Journal of Selected Topics in Signal Processing, 2007.).

The prediction function of LASSO model is as defined by Mathematical Formula 4.

$$\begin{matrix}p = \frac{1}{1 + {e}^{- β \cdot X}} = {logit}^{- 1} ( β \cdot X ) = {logit}^{- 1} ( {β}_{0} + {\sum }_{j = 1}^{m} {β}_{j} {x}_{j} ) & [ Mathematical Formula 4 ] \\ {min}_{β} λ {‖ β ‖}_{1} + {\sum }_{i = 1}^{n} log ( {e}^{- {y}_{i} ( β \cdot {X}_{i} )} + 1 ) & [ Mathematical Formula 5 ]\end{matrix}$$

The variables are as follows.

X is an independent variable of the model, accounting for a proportion of the gut microbiome in the fecal analysis data of infants.

β is a regression coefficient of the model and represents correlation between a microbe and a prediction variable.

p is a prediction score in the model and has a probability value between 0 and 1.

Xᵢ corresponds to a proportion of the microbiome in the n samples used for training, and yᵢ corresponds to the actual data from samples used (values of 0 and 1, respectively, depending on the actual variables used for grouping).

m is the number of the taxonomic groups of microbes used for training and has a natural value.

λ is a hyper parameter of the machine learning model.

In this regard, the first step is to set a regularization parameter, which is a weight to be used, according to the microbiome data. To this end, the process of selecting model parameters that give the best prediction result (the highest AUC value) was performed by equally cutting the regularization parameters into 10 models on an exponential scale between 0.0001 and 10000.

Through such a grid search, optimized hyperparameters could be obtained.

3-3. Screening of Biomarker by Group

The feature selection process was performed using the optimal model parameter obtained in Example 3-2. In the 100 rounds of learning replications, the frequency determined by the marker of each group is defined as robustness, and a value obtained by averaging the relevance (β) of each group is defined as a coefficient. The coefficient value indicates the influence of a biomarker, and also includes information on the group of which each biomarker is characteristic in each group.

In the case of a larger proportion of composition in each group, the coefficient values are distributed as negative and positive values, and are applied to the logistic function such as Mathematical Formula 4 to determine specificity for each group. It was set that a negative value is expressed for a case where a larger distribution is given to the first group and a positive value is expressed for a case where a larger distribution is given to the second group.

3-4. Feature Selection of Biomarker by Group

The LASSO application result of Example 3-3 is corrected according to the criteria for classifying each group to select a final microbial biomarker. For example, the microbes identified as biomarkers characteristic of the first group should show a higher proportion of the microbial taxa in the first group than in the second group. Therefore, the final biomarkers of the first group are selected by excluding the microbial taxa in which the proportion of the microbial taxa is higher in the second group. Through this process, biomarkers obtained by applying LASSO can be corrected according to the predefined criteria for dividing each group.

3-5. Model Verification Using Test Set

The test set selected through the 100 replications in Example 3-2 was applied to an optimized machine learning model. A prediction score for group identification can be calculated using the specific marker selected in Example 3-5 and the coefficient value of the marker.

When the coefficient of the microbes selected in Example 3-5 is \hat{β} and a proportion of the selected microbes in gut microflora is X′, the prediction score is calculated as shown in Mathematic Formula 6 below. In Mathematical Formula 6, the parameters are as defined above.

$$\hat{p} = {logit}^{- 1} ( \hat{β} \cdot {X}^{′} ) = \frac{1}{1 + {e}^{- \hat{β} \cdot {X}^{′}}} \qquad [ Mathematical Formula 6 ]$$

wherein,

\hat{β} is a coefficient of the selected microbes, and

X′ is a proportion of the selected microbes.

The prediction score is calculated as a value of 0 to 1 by finding the microbial marker selected from the gut microbiome data of the test set in Example 3-5 and multiplying the proportion of the microbial marker with the coefficient of the corresponding biomarker.

Verification can be made through the ROC curve (receiver operating characteristic curve) and AUC (area under curve) of the prediction model application result for the test set. It can be seen that the prediction model applied to the test set is significant by examining whether the ROC curve is greatly bent in a bow shape or whether the AUC value is close to 1.

3-6. Indexing of Prediction Model Determination Result

The prediction probability of the machine learning model is the probability calculated based on the determination results of the training set, but is not a probability accurately determined in the actual population. In order to give an accurate clinical interpretation to the prediction probability, the probability value between 0 and 1 was rescaled by dividing it by the ratio of the first group and the second group used for training. In Mathematical Formula 7, parameters are as defined above.

$$determination index = \frac{\hat{p}}{{p}_{o}} = \frac{\hat{p}}{{N}_{case} / {N}_{train}} \qquad [ Mathematical Formula 7 ]$$

wherein,

\hat{p} is a prediction score of a test subject for determining a specific group,

P₀ is a proportion of the second group samples present in the training set used to construct the prediction model,

N_{case} is the number of samples of the second group in the training set, and

Nₜᵣₐᵢₙ is the total number of samples in the training set.

Through the discrimination index obtained above, values of sensitivity, specificity, and accuracy are confirmed. Sensitivity refers to the proportion of the samples which are true positives to the second group out of the total number of samples in the second group, specificity refers to the proportion of the samples which are true negatives to the second group out of the total samples in the second group, and accuracy refers to a proportion of samples that are accurately determined to be in the first group or second group.

In detail, the cut-off of the discrimination index is determined by dividing the sensitivity, specificity, and accuracy values distributed in the entire samples into 20 equal parts. The sensitivity, the specificity, and the accuracy are calculated as shown by Mathematical Formulas 8 to 10, below. In Mathematical Formulas 8 to 10, the parameters are as defined above.

$$\begin{matrix}sensitivity = \frac{TP}{TP + FN} & [ Mathematical Formula 8 ] \\ specificity = \frac{TN}{TN + FP} & [ Mathematical Formula 9 ] \\ accuracy = \frac{TP + TN}{TP + TN + FP + FN} & [ Mathematical Formula 10 ]\end{matrix}$$

In Mathematical Formulas 8 to 10,

TP is the number of cases in which the determination index (\hat{p}) is greater than the cut-off in the samples corresponding to the second group,

TN is the number of cases in which the determination index (\hat{p}) is smaller than the cut-off in the samples corresponding to the second group,

FP is the number of cases in which the determination index (\hat{p}) is greater than the cut-off in the samples corresponding to the first group, and

FN is the number of cases in which the determination index (\hat{p}) is smaller than the cut-off in the samples corresponding to the first group.

When discrimination is made based on the index calculated to be the highest accuracy, accurate discrimination ability for the first or second group can be expected with the specificity or sensitivity accounted for by the index.

### Example 4. Determination of Developmental Stage in Infant

4-1. Classification of Infant Sample Through Dietary Step

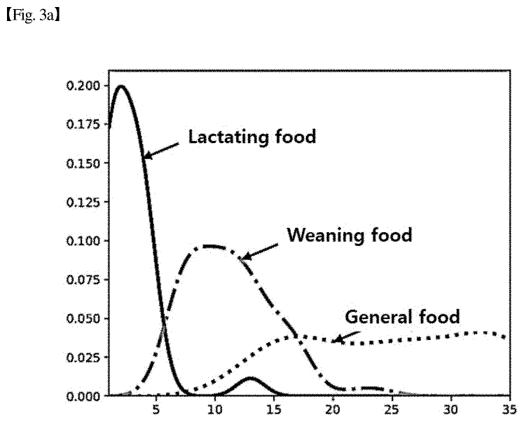

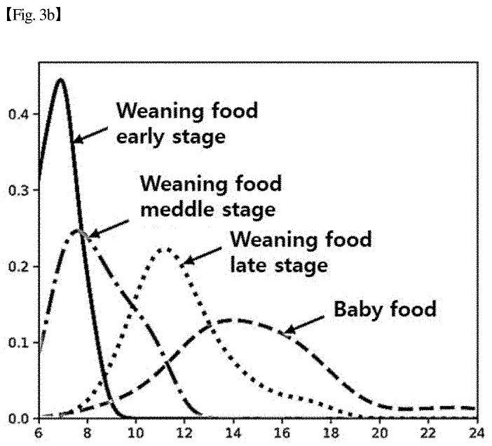

Based on the questionnaire designed to learn about the dietary habits of infants at the time of sample collection in Example 1, the distribution of feeding, weaning, and general foods in a total of 120 infant samples is shown in FIG. 3A and age (months) distribution was examined according to dietary step. The dietary steps of feeding, weaning, and general foods are defined according to the type of foods consumed by infants, and refer to the dietary mode in which infants consume liquid-type (feeding), gel-type (weaning), and solid-type (general) foods, respectively. According to FIG. 3B, the dietary steps are distributed in different patterns before and after 6 months and 15 months of age. The general food means the same solid diet as for an adult.

However, the dietary steps of infants aged 6 to 24 months are widely mixed. In order to examine the dietary steps of this period in detail, the dietary steps were divided into early-stage weaning food (brown), mid-stage weaning food (pink), late-stage weaning food (grey), and baby food (yellow), and examined in terms of months of age. For the step of weaning food, question 10 of the B-type questionnaire in Table 2 was used, and the results are depicted in FIG. 3B. According to FIG. 3B, distribution patterns of early, middle, and late weaning foods and baby foods are changed at about 15 months of age. According to the answers, the early-, mid-, and late-stage weaning foods and the baby food correspond to watery porridge (gel phase), thin porridge with smashed matter (gel phase), thick porridge (gel phase), and foods (solid phase) other than porridge, respectively.

Referring to FIGS. 3A and 3B, the samples of Example 1 can be divided into two groups in terms of diet and specifically into a group including feeding food (gel phase) and weaning food (gel phase) and a group including a baby food and a general food (solid phase).

Therefore, infants were defined to be in developmental stage 1 and developmental stage 2 according to the period of infants who ingest different types of foods including liquid and gel foods and solid foods. Distributions of the infant samples by diet step are summarized according to months of age in Table 5.

TABLE 5
Sample Distribution According to Infant Diet Step

| | Month | Month | Month | |
|---|---|---|---|---|
| Step | 0-10 | 11-14 | 15-36 | Sum |
| Feeding | 28 | 0 | 0 | 28 |
| Early weaning food | 4 | 0 | 0 | 4 |
| Mid weaning food | 18 | 1 | 0 | 19 |
| Late weaning food | 2 | 9 | 1 | 12 |
| Baby food | 1 | 3 | 7 | 11 |
| General food | 0 | 1 | 45 | 46 |
| Total | 53 | 14 | 53 | 120 |

4-2. Grouping of Developmental Stage Through Gut Microbe Analysis Data

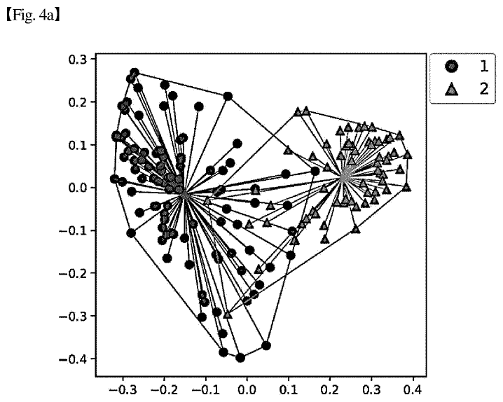

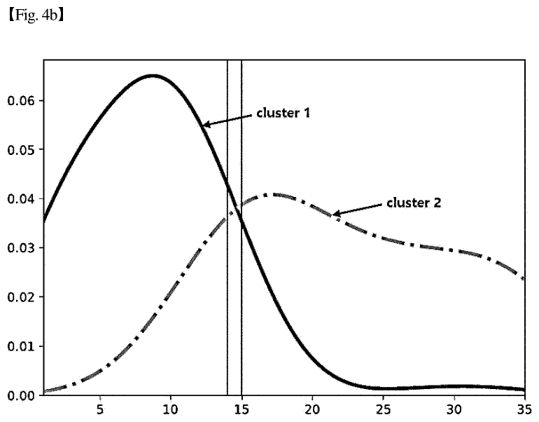

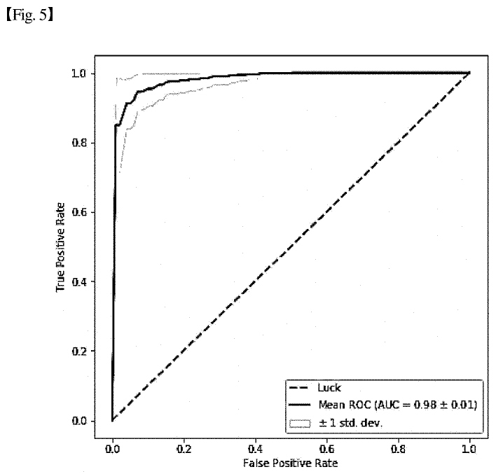

The entire infant samples were grouped using gut microbial data according to the DMM grouping method of Example 3-1, and the results are depicted in FIGS. 4A and 4B. According to DMM grouping, the entire infant samples were divided to a total of two developmental stage groups as shown in FIG. 4A. The first group included 69 samples while the second group included 51 samples. As a result of applying age (months post birth) to the first and second groups, the samples were divisionally distributed in two group patterns, based on about 15 months of age. The result is depicted in FIG. 4B. Therefore, the grouping results were observed to be in significant correlation with months of age in infants. With reference to Example 4-1, the first group and the second group were named developmental stage 1 and developmental stage 2, respectively.

Total number of samples: 120

| | |
|---|---|
| First group: developmental | Number of samples in first |
| stage 1 | group: 69 |
| Second group: developmental | Number of samples in second |
| stage 2 | group: 51 |

4-3. Application of Machine Learning Model According to Developmental Stage

Gut microbiome analysis data according to the developmental stages defined in Example 3-2 was applied to machine learning. The regularization parameter corresponding to a hyperparameter for the model, that is, the infant developmental stage prediction model optimized according to the present disclosure was selected as a value that allowed the best prediction result, among the λ values of Mathematical Formula 1. The optimized prediction value (hyperparameter) for determining the developmental stages was defined as 10.

4-4. Feature Selection of Biomarker by Developmental Stage Prediction Model (Primary)

According to the results of Example 4-3, selection was made of characteristic biomarkers that appeared primarily at each developmental stage. Biomarkers relevant to developmental stage 1 were found in 44 taxa at the species level and 12 taxa at the genus level. On the other hand, biomarkers relevant to developmental stage were found in 59 taxa at the species level and 22 taxa at the genus level. In Tables 6 to 9, biomarkers relevant to developmental stages 1 and 2 at species and genus levels are summarized.

In Tables 6 to 9, the coefficient corresponds to (3 of Mathematical Formula 4, and its negative and positive values mean microbes characteristic of developmental stage 1 and developmental stage 2, respectively. Robustness is the ratio of the number of times each microbe appeared at each developmental stage among 100 bootstrap replications, and robustness closer to 1 means that the microbe is more characteristic of the corresponding group. In addition, balance group and imbalance group proportions, which mean abundance ratio of respective microbiomes, are expressed as percentages of reads of corresponding microbes relative to a total number of reads of entire microbes.

The biomarkers in Tables 6 to 9 were primarily selected for classifying developmental stages of gut microbiomes in infants. In Tables 7 and 9, below, biomarkers at the genus level refer to microbial biomarkers which allow the discrimination of species, but are not specifically identified to the species level and thus mean microbial biomarkers discriminated at the species level.

TABLE 6
Developmental Stage 1-Related Biomarker at Species Level (Primary)

| | | | Balance | Imbalance |
|---|---|---|---|---|
| Sample No. | | | group | group |
| (species level) | | | proportion | proportion |
| (Microbe Name) | Coefficients | Robustness | (%) | (%) |
| Enterococcus | −0.618223 | 1 | 1.885624 | 0.01247 |
| faecalis | | | | |
| Streptococcus | −0.497649 | 0.95 | 0.414597 | 0.014475 |
| peroris | | | | |
| Bifidobacterium | −0.314397 | 1 | 28.477828 | 4.755615 |
| longum | | | | |
| Bifidobacterium | −0.244088 | 0.916667 | 1.498041 | 0.681195 |
| scardovii | | | | |
| Enterococcus | −0.201899 | 0.95 | 7.106151 | 1.48029 |
| faecium | | | | |
| Rothia | −0.152262 | 0.533333 | 0.177708 | 0.001652 |
| mucilaginosa | | | | |
| Veillonella parvula | −0.129975 | 0.716667 | 0.876127 | 0.032537 |
| Clostridioides | −0.061403 | 0.35 | 0.331878 | 0.100077 |
| difficile | | | | |
| Veillonella dispar | −0.056745 | 0.5 | 2.840242 | 0.776448 |
| Bifidobacterium | −0.055155 | 0.333333 | 0.102172 | 0.032258 |
| pseudolongum | | | | |
| Lactobacillus | −0.052383 | 0.3 | 0.662389 | 0.099017 |
| paracasei | | | | |
| Lactobacillus | −0.047133 | 0.3 | 0.182245 | 0.111444 |
| fermentum | | | | |
| Staphylococcus | −0.046645 | 0.266667 | 0.190544 | 0.003756 |
| aureus | | | | |
| Streptococcus | −0.03878 | 0.2 | 0.124871 | 0.123921 |
| sinensis | | | | |
| Lactobacillus | −0.036624 | 0.316667 | 0.034776 | 0.000525 |
| delbrueckii | | | | |
| Streptococcus | −0.029187 | 0.25 | 4.33701 | 2.858201 |
| salivarius | | | | |
| Clostridium | −0.024316 | 0.15 | 0.225459 | 0.042078 |
| paraputrificum | | | | |
| Bacteroides caccae | −0.024235 | 0.15 | 0.209986 | 0.211941 |
| Clostridium tertium | −0.023736 | 0.183333 | 0.243511 | 0.010923 |
| Bifidobacterium | −0.012494 | 0.15 | 0.226015 | 0.217978 |
| animalis | | | | |
| Clostridium | −0.012275 | 0.116667 | 0.172851 | 0.003612 |
| butyricum | | | | |
| Granulicatella | −0.011808 | 0.133333 | 0.032141 | 0.016157 |
| adiacens | | | | |
| FWNZ_s | −0.011279 | 0.116667 | 1.046689 | 0.030994 |
| (Genus Klebsiella) | | | | |
| Streptococcus | −0.010265 | 0.183333 | 1.033063 | 0.269234 |
| gallolyticus | | | | |
| Enterobacteriaceae | −0.009719 | 0.166667 | 1.469605 | 0.367475 |
| Bifidobacterium | −0.009272 | 0.183333 | 7.049017 | 1.053326 |
| breve | | | | |
| Clostridium | −0.009186 | 0.1 | 0.152768 | 0.005388 |
| perfringens | | | | |
| Escherichia coli | −0.007864 | 0.166667 | 6.548644 | 1.762086 |
| Terrisporobacter | −0.006267 | 0.083333 | 0.025722 | 0.080401 |
| petrolearius | | | | |
| Bacteroides vulgatus | −0.005773 | 0.1 | 1.839026 | 1.638793 |
| PAC001163_s | −0.004732 | 0.066667 | 0.336045 | 0.060607 |
| (Genus Blautia) | | | | |
| KQ235774_s | −0.004721 | 0.05 | 0.113268 | 0.106438 |
| (Genus Klebsiella) | | | | |
| Sutterella | −0.004474 | 0.05 | 0.027164 | 0.112334 |
| wadsworthensis | | | | |
| Clostridium | −0.002546 | 0.05 | 0.713797 | 0.236914 |
| ramosum | | | | |
| Bacteroides dorei | −0.001847 | 0.033333 | 0.2586 | 1.951592 |
| Prevotella copri | −0.001655 | 0.016667 | 0.087912 | 0.017899 |
| Veillonella atypica | −0.001547 | 0.033333 | 0.447658 | 0.065735 |
| Citrobacter koseri | −0.001237 | 0.016667 | 0.041994 | 0.023602 |
| CP011914_s | −0.00086 | 0.016667 | 0.013024 | 0.033725 |
| (Genus Eubacterium) | | | | |
| Clostridium celatum | −0.000719 | 0.016667 | 0.598248 | 1.369989 |
| PAC001178_s | −0.000559 | 0.016667 | 0.284282 | 0.030382 |
| (Genus Epulopiscium) | | | | |
| Collinsella | −0.000549 | 0.016667 | 0.210585 | 0.184206 |
| aerofaciens | | | | |
| Leuconostoc lactis | −0.000532 | 0.016667 | 0.033191 | 0.012436 |
| Bacteroides | −0.000289 | 0.033333 | 0.091656 | 1.41397 |
| uniformis | | | | |

TABLE 7
Developmental Stage 1-Related Biomarker at Genus Level (Primary)

| | | | Balance | Imbalance |
|---|---|---|---|---|
| Sample No. | | | group | group |
| (species level) | | | proportion | proportion |
| (Microbe Name) | Coefficients | Robustness | (%) | (%) |
| Enterococcus | −0.205616 | 0.983333 | 9.101523 | 1.514777 |
| Bifidobacterium | −0.13326 | 0.983333 | 40.409598 | 14.897967 |
| Streptococcus | −0.078202 | 0.733333 | 6.331705 | 3.528536 |
| Lactobacillus | −0.04119 | 0.633333 | 2.375967 | 0.931366 |
| Rothia | −0.013894 | 0.283333 | 0.191178 | 0.00428 |
| Veillonella | −0.005144 | 0.15 | 5.807609 | 2.012367 |
| Clostridioides | −0.004674 | 0.066667 | 0.342586 | 0.10047 |
| Enterobacteriaceae_g | −0.0043 | 0.1 | 1.469605 | 0.367475 |
| (Genus | | | | |
| Enterobacteriaceae) | | | | |
| Klebsiella | −0.002829 | 0.066667 | 1.051937 | 0.031341 |
| Actinomyces | −0.001029 | 0.05 | 0.306497 | 0.029024 |
| Clostridium | −0.000232 | 0.016667 | 2.230602 | 1.767117 |
| Staphylococcus | −0.000149 | 0.033333 | 0.191791 | 0.003814 |

TABLE 8
Developmental Stage 2-Related Biomarker at Species Level (Primary)

| | | | Balance | Imbalance |
|---|---|---|---|---|
| Sample No. | | | group | group |
| (species level) | | | proportion | proportion |
| (Microbe Name) | Coefficients | Robustness | (%) | (%) |
| Hungatella | 0.000251 | 0.016667 | 0.094561 | 0.073049 |
| hathewayi | | | | |
| Clostridium | 0.000519 | 0.016667 | 0.536483 | 0.496771 |
| innocuum | | | | |
| Blautia obeum | 0.000728 | 0.016667 | 0.025596 | 0.283337 |
| Roseburia | 0.000808 | 0.016667 | 0.033406 | 0.376978 |
| intestinalis | | | | |
| Clostridium | 0.000935 | 0.066667 | 0.561752 | 0.061132 |
| neonatale | | | | |
| Bacteroides ovatus | 0.001029 | 0.016667 | 0.170532 | 1.268938 |
| DQ799557_s | 0.001128 | 0.016667 | 0.079239 | 0.269888 |
| (Genus Bacteroides) | | | | |
| PAC001177_s | 0.001172 | 0.1 | 0.095936 | 0.16772 |
| (Family | | | | |
| Lachnospiraceae) | | | | |
| Coprobacillus | 0.001199 | 0.016667 | 0.032492 | 0.015334 |
| cateniformis | | | | |
| LT907848_s | 0.001287 | 0.016667 | 0.180159 | 0.692164 |
| (Genus | | | | |
| Anaerobutyricum) | | | | |
| PAC001143_s | 0.001374 | 0.016667 | 0.012706 | 0.27321 |
| (Genus | | | | |
| Eisenbergiella) | | | | |
| PAC001046_s | 0.001967 | 0.016667 | 0.000392 | 0.434514 |
| (Family | | | | |
| Lachnospiraceae) | | | | |
| PAC001305_s | 0.001981 | 0.033333 | 0.000453 | 0.177736 |
| (Family | | | | |
| Lachnospiraceae) | | | | |
| Intestinibacter | 0.001984 | 0.033333 | 0.534782 | 0.886648 |
| bartlettii | | | | |
| Bacteroides | 0.003104 | 0.05 | 0.043177 | 0.756637 |
| xylanisolvens | | | | |
| CCMM_s | 0.003396 | 0.1 | 0.126861 | 0.53309 |
| (Family | | | | |
| Erysipelotrichaceae) | | | | |
| KQ968618_s | 0.003604 | 0.066667 | 0.000306 | 0.666025 |
| (Genus Akkermansia) | | | | |
| Megasphaer | 0.003705 | 0.016667 | 0.033965 | 0.040222 |
| micronuciformis | | | | |
| Clostridium nexile | 0.003745 | 0.116667 | 0.591177 | 0.435947 |
| Roseburia | 0.003771 | 0.05 | 0.035658 | 0.738106 |
| inulinivorans | | | | |
| Ruminococcus | 0.004187 | 0.183333 | 3.57294 | 2.883981 |
| gnavus | | | | |
| Eggerthella lenta | 0.005097 | 0.066667 | 0.143669 | 0.123881 |
| Bifidobacterium | 0.005224 | 0.05 | 0.006449 | 1.546615 |
| adolescentis | | | | |
| Romboutsia | 0.005618 | 0.083333 | 0.666966 | 0.798258 |
| timonensis | | | | |
| Lactobacillus | 0.005688 | 0.083333 | 0.052894 | 0.563106 |
| rogosae | | | | |
| DQ799511_s | 0.006537 | 0.066667 | 0.002242 | 0.054269 |
| (Genus Blautia) | | | | |
| Clostridium | 0.008845 | 0.083333 | 0.124666 | 0.190663 |
| clostridioforme | | | | |
| Akkermansia | 0.011069 | 0.183333 | 0.605722 | 1.20175 |
| muciniphila | | | | |
| Cellulosilyticum | 0.011502 | 0.066667 | 0.022776 | 0.037526 |
| lentocellum | | | | |
| Parasutterella | 0.014228 | 0.1 | 0.001163 | 0.321641 |
| excrementihominis | | | | |
| Agathobaculum | 0.015198 | 0.116667 | 0.000282 | 0.165948 |
| butyriciproducens | | | | |
| Eubacterium hallii | 0.015404 | 0.2 | 0.094473 | 1.256658 |
| Faecalimonas | 0.016121 | 0.166667 | 0.049223 | 0.204978 |
| umbilicata | | | | |
| LN913006_s | 0.016775 | 0.133333 | 0.036213 | 0.425286 |
| (Genus Blautia) | | | | |
| Ruminococcus bromii | 0.019532 | 0.183333 | 0.015017 | 0.603128 |
| PAC001136_s | 0.021142 | 0.233333 | 0.004555 | 0.188245 |
| (Genus Clostridium) | | | | |
| Fusicatenibacter | 0.024694 | 0.216667 | 0.314287 | 1.907538 |
| saccharivorans | | | | |
| Ruminococcus faecis | 0.027194 | 0.15 | 0.124086 | 0.669968 |
| Bifidobacterium | 0.027944 | 0.316667 | 1.915472 | 5.482113 |
| catenulatum | | | | |
| Faecalibacterium | 0.03736 | 0.383333 | 0.98172 | 9.068555 |
| prausnitzii | | | | |
| Bacteroides fragilis | 0.049211 | 0.5 | 1.637415 | 5.670545 |
| Prevotella buccae | 0.049889 | 0.25 | 0.000156 | 0.560138 |
| Blautia faecis | 0.05423 | 0.35 | 0.000464 | 0.520471 |
| Sellimonas | 0.054853 | 0.366667 | 0.032135 | 0.243094 |
| intestinalis | | | | |
| Lactobacillus | 0.057454 | 0.4 | 0.600968 | 0.335825 |
| plantarum | | | | |
| PAC001048_s | 0.063003 | 0.35 | 0.002535 | 0.281593 |
| (Genus | | | | |
| Ruminococcaceae) | | | | |
| Roseburia cecicola | 0.072292 | 0.383333 | 0.108276 | 0.459604 |
| Clostridium | 0.096748 | 0.383333 | 0.07068 | 0.044219 |
| spiroforme | | | | |
| Veillonella ratti | 0.120458 | 0.7 | 1.346032 | 1.103508 |
| Agathobacter rectalis | 0.133908 | 0.666667 | 0.057285 | 0.579458 |
| Clostridium | 0.13445 | 0.65 | 0.145693 | 0.118909 |
| symbiosum | | | | |
| Anaerostipes hadrus | 0.171178 | 0.816667 | 0.304226 | 3.597979 |
| Gemmiger formicilis | 0.175736 | 0.75 | 0.080487 | 1.546753 |
| Alistipes onderdonkii | 0.202069 | 0.616667 | 0.000477 | 0.411531 |
| Blautia hansenii | 0.271272 | 0.883333 | 0.151363 | 0.166795 |
| PAC001148_s | 0.28366 | 0.933333 | 0.271439 | 0.640186 |
| (Family | | | | |
| Lachnospiraceae) | | | | |
| Bifidobacterium | 0.309518 | 0.916667 | 0.87911 | 0.874891 |
| bifidum | | | | |
| Ruminococcus | 0.379602 | 0.866667 | 0.002731 | 0.228165 |
| torques | | | | |
| Blautia wexlerae | 0.660876 | 1 | 0.634202 | 6.432939 |

TABLE 9
Developmental Stage 2-Related Biomarker at Genus Level (Primary)

| Sample No. (species level) | | | Balance group | Imbalance group |
|---|---|---|---|---|
| (Microbe Name) | Coefficients | Robustness | proportion (%) | proportion (%) |
| Coprococcus_g2 | 0.000345 | 0.033333 | 0.634298 | 0.518112 |
| (Family Lachnospiraceae) | | | | |
| Prevotella | 0.000503 | 0.033333 | 0.728 | 1.326638 |
| Agathobacter | 0.000716 | 0.066667 | 0.057737 | 0.59893 |
| PAC000672_g | 0.00116 | 0.033333 | 0.002775 | 0.282116 |
| (Family Ruminococcaceae) | | | | |
| Pseudoflavonifractor | 0.001503 | 0.05 | 0.159467 | 0.221574 |
| Lachnospira | 0.001773 | 0.016667 | 0.316929 | 1.607492 |
| Eubacterium_g5 | 0.001984 | 0.05 | 0.278224 | 2.044322 |
| (Family Lachnospiraceae) | | | | |
| Alistipes | 0.00218 | 0.05 | 0.000794 | 0.539207 |
| Clostridium_g24 | 0.003903 | 0.1 | 0.479107 | 1.181915 |
| (Family Lachnospiraceae) | | | | |
| Akkermansia | 0.004259 | 0.1 | 0.607531 | 1.890071 |
| Ruminococcus_g5 | 0.005001 | 0.133333 | 3.606215 | 2.937409 |
| (Family Lachnospiraceae) | | | | |
| Roseburia | 0.021967 | 0.416667 | 0.179598 | 1.660123 |
| Fusicatenibacter | 0.022191 | 0.366667 | 0.317638 | 1.958465 |
| Sellimonas | 0.0305 | 0.5 | 0.033008 | 0.279463 |
| Ruminococcus_g2 | 0.033029 | 0.483333 | 0.020473 | 1.177464 |
| (Family Ruminococcaceae) | | | | |
| Bacteroides | 0.03449 | 0.7 | 4.901392 | 16.590086 |
| Eisenbergiella | 0.036326 | 0.4 | 0.042349 | 0.415958 |
| Subdoligranulum | 0.043866 | 0.65 | 0.145427 | 1.898761 |
| Ruminococcus_g4 | 0.054933 | 0.683333 | 0.178463 | 1.097635 |
| (Family Lachnospiraceae) | | | | |
| Anaerostipes | 0.146638 | 0.883333 | 0.552492 | 3.926105 |
| Faecalibacterium | 0.153494 | 0.95 | 0.98321 | 9.194765 |
| Blautia | 0.326798 | 1 | 1.464729 | 8.879734 |

4-5. Feature Selection of Biomarker by Developmental Stage Prediction Model (Secondary)

The characteristic biomarkers primarily selected by the method of Example 4-4 were corrected by the method described in Examples 3-4. In brief, a total of 7 microbial species, such as Bacteroides caccae, Terrisporobacter pertrolearius, etc., which showed larger proportions of microbial taxa in developmental stage 2, were excluded out of the species-level biomarkers characteristic of developmental stage 1 in Table 6. In addition, a total of 14 microbial species such as Lachnospiraceae, unpublished species, Clostridium innocuum, Hungatella hathewayi, which showed larger proportions of microbial taxa in developmental stage 1, were excluded out of biomarkers characteristic of developmental stage 2 in Table 8.

In consideration of the excluded microbial taxa, characteristic biomarkers for each developmental stage are shown in Tables 10 to 13, below. The biomarkers of Tables 10 to 13 below are the final biomarkers selected secondarily as biomarkers for discriminating the developmental stages of gut microbiomes in infants. Biomarkers characteristic of developmental stage 1 were found in 37 taxa at the species level and 12 taxa at the genus level. On the other hand, biomarkers characteristic of developmental stage were found in 47 taxa at the species level and 20 taxa at the genus level. In Tables 6 to 9, biomarkers relevant to developmental stages 1 and 2 at species and genus levels are summarized. In Tables 11 and 13, below, biomarkers at the genus level refer to microbial biomarkers which allow the discrimination of species, but are not specifically identified to the species level and thus mean microbial biomarkers discriminated at the species level.

TABLE 10
Developmental Stage 1-Related Biomarker (Secondary)

| | | | | Imbalance |
|---|---|---|---|---|
| Sample No. (species level) | | | Balance group | group |
| (Microbe Name) | Coefficients | Robustness | proportion (%) | proportion (%) |
| Enterococcus faecalis | −0.618223 | 1 | 1.885624 | 0.01247 |
| Streptococcus peroris | −0.497649 | 0.95 | 0.414597 | 0.014475 |
| Bifidobacterium longum | −0.314397 | 1 | 28.477828 | 4.755615 |
| Bifidobacterium scardovii | −0.244088 | 0.916667 | 1.498041 | 0.681195 |
| Enterococcus faecium | −0.201899 | 0.95 | 7.106151 | 1.48029 |
| Rothia mucilaginosa | −0.152262 | 0.533333 | 0.177708 | 0.001652 |
| Veillonella parvula | −0.129975 | 0.716667 | 0.876127 | 0.032537 |
| Clostridioides difficile | −0.061403 | 0.35 | 0.331878 | 0.100077 |
| Veillonella dispar | −0.056745 | 0.5 | 2.840242 | 0.776448 |
| Bifidobacterium | −0.055155 | 0.333333 | 0.102172 | 0.032258 |
| pseudolongum | | | | |
| Lactobacillus paracasei | −0.052383 | 0.3 | 0.662389 | 0.099017 |
| Lactobacillus fermentum | −0.047133 | 0.3 | 0.182245 | 0.111444 |
| Staphylococcus aureus | −0.046645 | 0.266667 | 0.190544 | 0.003756 |
| Streptococcus sinensis | −0.03878 | 0.2 | 0.124871 | 0.123921 |
| Lactobacillus delbrueckii | −0.036624 | 0.316667 | 0.034776 | 0.000525 |
| Streptococcus salivarius | −0.029187 | 0.25 | 4.33701 | 2.858201 |
| Clostridium paraputrificum | −0.024316 | 0.15 | 0.225459 | 0.042078 |
| Clostridium tertium | −0.023736 | 0.183333 | 0.243511 | 0.010923 |
| Bifidobacterium animalis | −0.012494 | 0.15 | 0.226015 | 0.217978 |
| Clostridium butyricum | −0.012275 | 0.116667 | 0.172851 | 0.003612 |
| Granulicatella adiacens | −0.011808 | 0.133333 | 0.032141 | 0.016157 |
| FWNZ_s (Genus Klebsiella) | −0.011279 | 0.116667 | 1.046689 | 0.030994 |
| Streptococcus gallolyticus | −0.010265 | 0.183333 | 1.033063 | 0.269234 |
| Enterobacteriaceae | −0.009719 | 0.166667 | 1.469605 | 0.367475 |
| Bifidobacterium breve | −0.009272 | 0.183333 | 7.049017 | 1.053326 |
| Clostridium perfringens | −0.009186 | 0.1 | 0.152768 | 0.005388 |
| Escherichia coli | −0.007864 | 0.166667 | 6.548644 | 1.762086 |
| Bacteroides vulgatus | −0.005773 | 0.1 | 1.839026 | 1.638793 |
| PAC001163_s (Genus Blautia) | −0.004732 | 0.066667 | 0.336045 | 0.060607 |
| KQ235774_s (Genus | −0.004721 | 0.05 | 0.113268 | 0.106438 |
| Klebsiella) | | | | |
| Clostridium ramosum | −0.002546 | 0.05 | 0.713797 | 0.236914 |
| Prevotella copri | −0.001655 | 0.016667 | 0.087912 | 0.017899 |
| Veillonella atypica | −0.001547 | 0.033333 | 0.447658 | 0.065735 |
| Citrobacter koseri | −0.001237 | 0.016667 | 0.041994 | 0.023602 |
| PAC001178_s (Genus | −0.000559 | 0.016667 | 0.284282 | 0.030382 |
| Epulopiscium) | | | | |
| Collinsella aerofaciens | −0.000549 | 0.016667 | 0.210585 | 0.184206 |
| Leuconostoc lactis | −0.000532 | 0.016667 | 0.033191 | 0.012436 |

TABLE 11
Developmental Stage 1-Related Biomarker at Genus Level (Secondary)

| | | | | Imbalance |
|---|---|---|---|---|
| Sample No. (species level) | | | Balance group | group |
| (Microbe Name) | Coefficients | Robustness | proportion (%) | proportion (%) |
| Enterococcus | −0.205616 | 0.983333 | 9.101523 | 1.514777 |
| Bifidobacterium | −0.13326 | 0.983333 | 40.409598 | 14.897967 |
| Streptococcus | −0.078202 | 0.733333 | 6.331705 | 3.528536 |
| Lactobacillus | −0.04119 | 0.633333 | 2.375967 | 0.931366 |
| Rothia | −0.013894 | 0.283333 | 0.191178 | 0.00428 |
| Veillonella | −0.005144 | 0.15 | 5.807609 | 2.012367 |
| Clostridioides | −0.004674 | 0.066667 | 0.342586 | 0.10047 |
| Enterobacteriaceae_g (Genus | −0.0043 | 0.1 | 1.469605 | 0.367475 |
| Enterobacteriaceae) | | | | |
| Klebsiella | −0.002829 | 0.066667 | 1.051937 | 0.031341 |
| Actinomyces | −0.001029 | 0.05 | 0.306497 | 0.029024 |
| Clostridium | −0.000232 | 0.016667 | 2.230602 | 1.767117 |
| Staphylococcus | −0.000149 | 0.033333 | 0.191791 | 0.003814 |

TABLE 12
Developmental Stage 2-Related Biomarker at Species Level (Secondary)

| | | | Balance | |
|---|---|---|---|---|
| | | | group | |
| Sample No. (species level) | | | proportion | Imbalance group |
| (Microbe Name) | Coefficients | Robustness | (%) | proportion (%) |
| Blautia obeum | 0.000728 | 0.016667 | 0.025596 | 0.283337 |
| Roseburia intestinalis | 0.000808 | 0.016667 | 0.033406 | 0.376978 |
| Bacteroides ovatus | 0.001029 | 0.016667 | 0.170532 | 1.268938 |
| DQ799557_s | 0.001128 | 0.016667 | 0.079239 | 0.269888 |
| (Genus Bacteroides) | | | | |
| PAC001177_s | 0.001172 | 0.1 | 0.095936 | 0.16772 |
| (Family Lachnospiraceae) | | | | |
| LT907848_s | 0.001287 | 0.016667 | 0.180159 | 0.692164 |
| (Genus Anaerobutyricum) | | | | |
| PAC001143_s | 0.001374 | 0.016667 | 0.012706 | 0.27321 |
| (Genus Eisenbergiella) | | | | |
| PAC001046_s | 0.001967 | 0.016667 | 0.000392 | 0.434514 |
| (Family Lachnospiraceae) | | | | |
| PAC001305_s | 0.001981 | 0.033333 | 0.000453 | 0.177736 |
| (Family Lachnospiraceae) | | | | |
| Intestinibacter bartlettii | 0.001984 | 0.033333 | 0.534782 | 0.886648 |
| Bacteroides xylanisolvens | 0.003104 | 0.05 | 0.043177 | 0.756637 |
| CCMM_s | 0.003396 | 0.1 | 0.126861 | 0.53309 |
| (Family Erysipelotrichaceae) | | | | |
| KQ968618_s | 0.003604 | 0.066667 | 0.000306 | 0.666025 |
| (Genus Akkermansia) | | | | |
| Megasphaera micronuciformis | 0.003705 | 0.016667 | 0.033965 | 0.040222 |
| Roseburia inulinivorans | 0.003771 | 0.05 | 0.035658 | 0.738106 |
| Bifidobacterium adolescentis | 0.005224 | 0.05 | 0.006449 | 1.546615 |
| Romboutsia timonensis | 0.005618 | 0.083333 | 0.666966 | 0.798258 |
| Lactobacillus rogosae | 0.005688 | 0.083333 | 0.052894 | 0.563106 |
| DQ799511_s (Genus Blautia) | 0.006537 | 0.066667 | 0.002242 | 0.054269 |
| Clostridium clostridioforme | 0.008845 | 0.083333 | 0.124666 | 0.190663 |
| Akkermansia muciniphila | 0.011069 | 0.183333 | 0.605722 | 1.20175 |
| Cellulosilyticum lentocellum | 0.011502 | 0.066667 | 0.022776 | 0.037526 |
| Parasutterella | 0.014228 | 0.1 | 0.001163 | 0.321641 |
| excrementihominis | | | | |
| Agathobaculum | 0.015198 | 0.116667 | 0.000282 | 0.165948 |
| butyriciproducens | | | | |
| Eubacterium hallii | 0.015404 | 0.2 | 0.094473 | 1.256658 |
| Faecalimonas umbilicata | 0.016121 | 0.166667 | 0.049223 | 0.204978 |
| LN913006_s (Genus Blautia) | 0.016775 | 0.133333 | 0.036213 | 0.425286 |
| Ruminococcus bromii | 0.019532 | 0.183333 | 0.015017 | 0.603128 |
| PAC001136_s (Genus | 0.021142 | 0.233333 | 0.004555 | 0.188245 |
| Clostridium) | | | | |
| Fusicatenibacter saccharivorans | 0.024694 | 0.216667 | 0.314287 | 1.907538 |
| Ruminococcus faecis | 0.027194 | 0.15 | 0.124086 | 0.669968 |
| Bifidobacterium catenulatum | 0.027944 | 0.316667 | 1.915472 | 5.482113 |
| Faecalibacterium prausnitzii | 0.03736 | 0.383333 | 0.98172 | 9.068555 |
| Bacteroides fragilis | 0.049211 | 0.5 | 1.637415 | 5.670545 |
| Prevotella buccae | 0.049889 | 0.25 | 0.000156 | 0.560138 |
| Blautia faecis | 0.05423 | 0.35 | 0.000464 | 0.520471 |
| Sellimonas intestinalis | 0.054853 | 0.366667 | 0.032135 | 0.243094 |
| PAC001048_s | 0.063003 | 0.35 | 0.002535 | 0.281593 |
| (Genus Lachnospiraceae) | | | | |
| Roseburia cecicola | 0.072292 | 0.383333 | 0.108276 | 0.459604 |
| Agathobacter rectalis | 0.133908 | 0.666667 | 0.057285 | 0.579458 |
| Anaerostipes hadrus | 0.171178 | 0.816667 | 0.304226 | 3.597979 |
| Gemmiger formicilis | 0.175736 | 0.75 | 0.080487 | 1.546753 |
| Alistipes onderdonkii | 0.202069 | 0.616667 | 0.000477 | 0.411531 |
| Blautia hansenii | 0.271272 | 0.883333 | 0.151363 | 0.166795 |
| PAC001148_s | 0.28366 | 0.933333 | 0.271439 | 0.640186 |
| (Family Lachnospiraceae) | | | | |
| Ruminococcus torques | 0.379602 | 0.866667 | 0.002731 | 0.228165 |
| Blautia wexlerae | 0.660876 | 1 | 0.634202 | 6.432939 |

TABLE 13
Developmental Stage 2-Related Biomarker at Genus Level (Secondary)

| | | | Balance group | Imbalance |
|---|---|---|---|---|
| Sample No. (species level) | | | proportion | group |
| (Microbe Name) | Coefficients | Robustness | (%) | proportion (%) |
| Prevotella | 0.000503 | 0.033333 | 0.728 | 1.326638 |
| Agathobacter | 0.000716 | 0.066667 | 0.057737 | 0.59893 |
| PAC000672_g | 0.00116 | 0.033333 | 0.002775 | 0.282116 |
| (Family Ruminococcaceae) | | | | |
| Pseudoflavonifractor | 0.001503 | 0.05 | 0.159467 | 0.221574 |
| Lachnospira | 0.001773 | 0.016667 | 0.316929 | 1.607492 |
| Eubacterium_g5 | 0.001984 | 0.05 | 0.278224 | 2.044322 |
| (Family Lachnospiraceae) | | | | |
| Alistipes | 0.00218 | 0.05 | 0.000794 | 0.539207 |
| Clostridium_g24 | 0.003903 | 0.1 | 0.479107 | 1.181915 |
| (Family Lachnospiraceae) | | | | |
| Akkermansia | 0.004259 | 0.1 | 0.607531 | 1.890071 |
| Roseburia | 0.021967 | 0.416667 | 0.179598 | 1.660123 |
| Fusicatenibacter | 0.022191 | 0.366667 | 0.317638 | 1.958465 |
| Sellimonas | 0.0305 | 0.5 | 0.033008 | 0.279463 |
| Ruminococcus_g2 | 0.033029 | 0.483333 | 0.020473 | 1.177464 |
| (Family Ruminococcaceae) | | | | |
| Bacteroides | 0.03449 | 0.7 | 4.901392 | 16.590086 |
| Eisenbergiella | 0.036326 | 0.4 | 0.042349 | 0.415958 |
| Subdoligranulum | 0.043866 | 0.65 | 0.145427 | 1.898761 |
| Ruminococcus_g4 | 0.054933 | 0.683333 | 0.178463 | 1.097635 |
| (Family Lachnospiraceae) | | | | |
| Anaerostipes | 0.146638 | 0.883333 | 0.552492 | 3.926105 |
| Faecalibacterium | 0.153494 | 0.95 | 0.98321 | 9.194765 |
| Blautia | 0.326798 | 1 | 1.464729 | 8.879734 |

4-6. Verification of Infant Developmental Stage Prediction Model

Using the method of Example 3-5, it was examined whether the optimized machine learning model that had been trained for infant developmental stages can accurately distinguish the infant developmental stages.

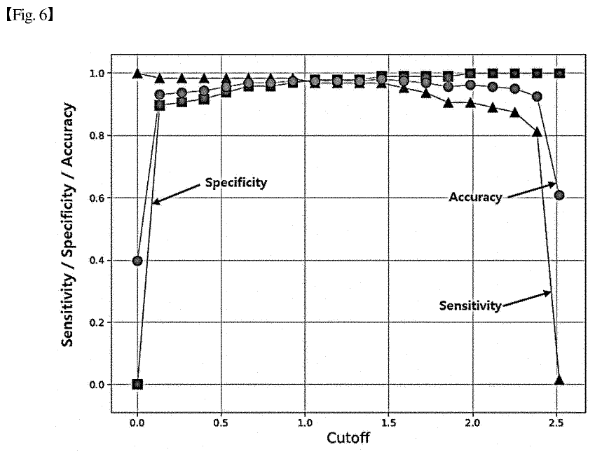

The ROC curve (receiver operating characteristic curve) and AUC (area under curve) as a result of application of the optimized machine learning model to determining developmental stages for the test sets are depicted in FIG. 6. As shown, the ROC curve is greatly bent in a bow shape or the AUC value is close to 1, thus demonstrating that the prediction results for infant developmental stages in Example 4-3 are significant.

4-7. Determination Index of Infant Developmental Stage

In order to give an accurate clinical interpretation to the prediction result of Example 4-6, the probability value between 0 and 1 calculated by multiplying the of the microbial marker with the coefficient of the corresponding biomarker was rescaled by dividing it by the ratio of the developmental stage 1 and the developmental stage 2 used for training

In Mathematical Formula 7,

\hat{p} is a prediction score of a test subject for determining developmental stage 2,

P₀ is a proportion of samples, corresponding to developmental stage 2, present in the training set used to construct the prediction model,

N_{case} is the number of samples corresponding to developmental stage 2 in the training set, and

Nₜᵣₐᵢₙ is the total number of samples in the training set. The calculated index is named “infant development index”.

Through the infant development index, the sensitivity, the specificity, and the accuracy were checked. In Mathematical Formulas 8 to 10, FP is the number of cases in which the infant development index (\hat{p}) is greater than the cut-off in the samples corresponding to developmental stage 1, FN is the number of cases in which the infant development index (\hat{p}) is smaller than the cut-off in the samples corresponding to the developmental stage 1, TP is the number of cases in which the infant development index (\hat{p}) is greater than the cut-off in the samples corresponding to developmental stage 2, and TN is the number of cases in which the infant development index (\hat{p}) is smaller than the cut-off in the samples corresponding to the developmental stage 2. These results are depicted in FIG. 6.

When infant developmental stages were determined on the basis of the highest index 1.19 at which accuracy was calculated to be about 98%, the specificity which indicates the probability of infant developmental stage 2 was about 98% and the sensitivity which indicates the probability of infant developmental stage 1 was about 97%. The accuracy plot is given in FIG. 6.

When dysbiosis was determined on the basis of the infant development index of 1.19, the specificity was measured to be about 98% and can accurately discriminate the developmental stages, demonstrating its high clinical discriminating potential. Therefore, when the infant development index is 1.19 or greater, the infant can be determined to be in developmental stage 2. When the infant development index is calculated to be below 1.19, the infant can be determined to be in developmental stage 1.

4-8. Classification of Gut Microbe Developmental Stage in Infant

(A) Introduction of Classification of Developmental Stage in Infant

Developmental stages in infants can be determined in terms of at least one reference selected from the group consisting of dietary stage, months of age, and infant development index (based on information on gut microbiome). With respect to the infant development index, the biomarkers in Tables 10 to 13 below are biomarkers that classify the developmental stage of gut microbiome of infants, and use the final biomarkers secondly selected. In Table 14 below, methods for determining developmental stages according to determination criteria for the developmental stages are summarized

TABLE 14
Classification Reference for Infant Developmental Stage

| | | Developmental |
|---|---|---|
| Category | Developmental Stage 1 | Stage 2 |
| Dietary step | Feeding diet (liquid-type food), | Solid diet |
| | Weaning diet (gel-type food) | |
| Age (months | Below 15 months | 15 months or |
| after birth) | | older |
| Infant develop- | Less than 1.19 | 1.19 or greater |
| ment index | | |

(B) Classification of Infant Developmental Stage in Terms of Dietary Step

The classification of infant developmental stages through dietary steps is designed to divide diets of infants into liquid-type feeding foods, gel-type weaning foods, solid-type infant foods, and solid-type general foods, and to set the dietary step of liquid-type feeding foods or gel-type weaning foods as developmental stage 1 and the dietary step of solid-type foods, that is, infant foods or general foods as developmental stage 2 on the basis of the metadata information (diet) of infants. Thus, the time at which infants fed with liquid- or gel-type feeding foods or weaning foods ingest a solid-type food is a criterion for infant developmental stages.

(C) Classification of Infant Developmental Stage in Terms of Months of Age

For classification of developmental stages according to age (months after birth), developmental stage 1 is set for a test infant who is under 15 months after birth and developmental stage 2 is set for a test infant who is at 15 months or more of age.

The criterion of 15 months was defined with reference to the time when the diet type is converted from gel-type to solid-type foods and the time when the data groups were classified through the DMM grouping method of Example 4-2. Therefore, the criterion of 15 months defined by the above method means the time when the dietary steps are most clearly divided and when microbial kinds and their respective abundance ratio in gut microbiome are most greatly changed. Infant gut microbes consist mainly of microbes that contribute to immunity, digestion of breast milk, and intestinal stabilization, immediately after birth, and exhibit a greatly increased spectrum of microbial kinds with the predominance of microbes associated with metabolisms of various foods, such as dietary fibers, etc., since the time of 15 months after birth.

(D) Classification of Infant Developmental Stage in Terms of Biomarker Characteristic of Developmental Stage

In a case where an infant development index is adopted as a criterion, kinds (species) of respective microbial biomarkers characteristic of developmental stages and a proportion (abundance ratio) of the characteristic species in gut microflora are analyzed on the basis of the microbiome analysis data for the collected gut microbes and applied to the above-mentioned infant developmental stage prediction model to classify the developmental stage. Species of microbial biomarkers characteristic of each developmental stage and a proportion (abundance ratio) of the species in gut microflora are analyzed to calculate a development index. A cut-off value is set for the development index in terms of accuracy, sensitivity, and specificity. Developmental stage 1 is given to a case where the development index is less than the cut-off value while developmental stage 2 is given to a case where the development index is as high as or higher than the cut-off value.

In the present disclosure, as explained in Examples 4-7, decision is made of developmental stage 1 for a measurement less than the development index 1.19 and developmental stage 2 for a measurement as high as or higher than the development index 1.19.

When developmental stages are classified according to answers to a questionnaire for dietary steps and months after birth, answers to the questionnaire including items of Table 2 should take precedence. For classification on the basis of the infant development index, analysis of gut microbes using the method of Example 2 should take precedence.

The gut microbial ecosystem of infants is established as microbes residing in parents and surrounding environments are transferred to and settled down in newborns free of germs, and the abundance and diversity of microbial species in infants increase with their growth and diet. In this increasing trend, biomarkers characteristic of developmental stages of infants account specifically for the development pattern of the intestinal microbial ecosystem according to the growth of infants. Biomarkers characteristic of developmental stage 1 are given in Tables 10 and 11, while biomarkers characteristic of developmental stage 2 are listed in Tables 12 and 13.

Among biomarkers characteristic of developmental stage 1, Enterococcus, Streptococcus, and Lactobacillus are microbes belong to the Firmicutes phylum and Bifidobacterium is a microbe belonging to the Actinobacteria phylum. Enterococcus, Bifidobacterium, Streptococcus, and Lactobacillus are all lactic acid bacteria. Lactic acid bacteria are microbes that are transmitted from the mother's body to her child and first settles more easily in the germ-free intestines of the newborn than other microbes and then secretes antibacterial substances to prevent the settlement of various external antigens. Also, Bifidobacterium longum, which is one of the species-level biomarkers characteristic of developmental stage 1, contributes to immunity through the mechanism in which proteolytic enzymes expressed in foreign antigens are inhibited by a substance called serpin. Above all, lactic acid bacteria are microbes that break down lactose, and play the biggest role in helping infants digest breast milk well. Escherichia spp., which belong to the phylum Proteobacteria, are microbes most abundantly found in infant feces before transmission of lactic acid bacteria. The microbes settle first in the intestinal environment of newborns and absorb oxygen in the intestine, thereby creating an anaerobic environment in the intestine and contributing to the stabilization of the intestinal environment.

The biomarkers characteristic of developmental stage 2 may be largely divided into the Firmicutes phylum including Blautia, Faecalibacterium, and Anaerostipes and the Bacteroidetes phylum including Prevotella and Bacteroides.

It can be seen that the gut microbiome was converted into Firmicutes and Bacteroidetes phyla from the Firmicutes and Actinobacteria phyla in which lactic acid bacteria are predominant (developmental stage 1). The noticeable occupancy of the Firmicutes and Bacteroidetes phyla in the gut microbiome is the most common feature in adults. This pattern means that the gut microbial ecosystem of infants is developing in a similar way to that of adults. As infants grow physically and require a variety of nutrients, the food they consume also diversifies, increasing the microbial species of the gut microbiome and the metabolic diversity of each microbial species.

As stated in Example 2-2, the biomarkers characteristic of development stage 2 are mainly composed of short-chain fatty acid producing bacteria associated with fiber metabolism. Particularly in the case of the Firmicutes phylum in developmental stage 2, the microbial species (Blautia, Faecalibacterium, Anaerostipes) of the Clostridiales order, which contain the most abundant representative short-chain fatty acid producing bacteria, show the highest coefficient values.

Microbes in Prevotella and Bacteroides genera of the Bacteroidetes phylum are representative microbes that degrade dietary fibers and proteins. These microbes were described as a criterion for dividing enterotypes of adults covering races, regions, and individuals in the journal Nature, 2011. In a follow-up study on enterotypes, it was reported that Prevotella was mainly found upon ingestion of high-fiber-low-protein diets, and Bacteroides appeared more frequently upon ingestion of low-fiber (simple sugar)-high-protein diets. Referring to coefficient and robustness values of Prevotella and Bacteroides in development stage 2, Bacteroides was higher in both coefficient and robustness than Prevotella. From the data, it is understood that gut microbe types in infants develop predominantly into the Bacteroides type. However, studies still remain insufficient to accurately identify the types of Prevotella and Bacteroides, and there are still different interpretations of enterotypes that are mainly seen in the gut microflora of infants.

### Example 5. Classification of Imbalance and Balance of Gut Microbe by Infant Developmental Stage

5-1. Grouping of Gut Microbe Analysis Data by Developmental Stage

The entire infant samples were grouped for each developmental stage using gut microbe analysis data according to the DMM grouping method of Example 3-1. In each developmental stage, the samples were divided into two groups, and a total of 4 groups were grouped. In each developmental stage, the infant samples were distributed as follows.

Total number of samples: 120

No. of samples in 1^{st} group of developmental stage 1: 36

No. of samples in 2^{nd} group of developmental stage 1: 33

No. of samples in 3^{rd} group of developmental stage 2: 32

No. of samples in 4^{th} group of developmental stage 2: 19

5-2. Determination of Infant Dysbiosis by Using Infant Metadata

In order to calculate correlation with factors associated with gut microbial imbalance and balance, reference was made to the study by Chong, 2018 (Factors Affecting Gastrointestinal Microbiome Development in Neonates. Nutrients. 2018 Feb. 28; 10(3).).

Gut microbe imbalance is generally defined as an imbalanced state caused by the consumption of processed foods and the use of antibiotics, which are factors that reduce the species diversity of the gut ecosystem. Although there are no clear criteria for the definition thereof, the gut microbe balance, as used herein, is defined as a gut microbial ecosystem in a healthy infant sample that does not contain any imbalanced factor. Therefore, the gut microbe imbalance group is defined as a sample group possessing a gut microbiome associated with metadata causative of gut imbalance and gut balance group is defined as a sample group possessing a gut microbiome associated with metadata alleviative of gut imbalance. Factors related to gut imbalance and balance were selected referring to the metadata collected with the questionnaire in Table 2 and the items stated in Chong's 2018 study. Factors that affect gut microflora of newborns include the age of infants, whether or not antibiotics are taken, the mode of delivery, feeding methods, and the presence or absence of diarrhea. The selected items and answers are classified in Table 15.

TABLE 15

| | | | Metadata |
|---|---|---|---|
| Questionnaire | Metadata item | Answer option | classification |
| Please write your | age_month | (months of age) | age_month |
| child's information | | | |
| What was the mode | birth_mode_ | \textcircled{1} natural delivery | birth_mode_naturalTRUE |
| of delivery when the | natural | | |
| child was born? | | \textcircled{2} cesarean section \textcircled{3}? cesarean | birth_mode_naturalFALSE |
| | | section after failure of natural | |
| | | delivery | |
| What is the lactation | lactation_bf | \textcircled{1} breastfeeding | lactation_bfTRUE |
| method? If you are | | \textcircled{2} formula feeding | lactation_bfFALSE |
| using formula | | \textcircled{3} mixed feeding | |
| feeding or mixed | | | |
| feeding, please write | | | |
| down the name of | | | |
| the product you are | | | |
| currently using. | | | |
| Has your child taken | Antibiotics | \textcircled{1} yes | antibioticsTRUE |
| antibiotics in the past | | \textcircled{2} no | antibioticsFALSE |
| month? | | | |
| (A) Which of the | diarrhea | \textcircled{1} diarrhea for many cases | diarrheaTRUE |
| following is the | | \textcircled{2} general loose stool | diarrheaFALSE |
| child's common stool | | | |
| form over the past | | | |
| month? | | | |
| (B, C) Which of the | diarrhea | \textcircled{6} mild diarrhea (feces that are | diarrheaTRUE |
| following is the | | very watery and come out like | |
| child's common stool | | mud) | |
| form over the past | | \textcircled{7}? severe diarrhea (feces that | |
| month? | | come out like water) | |
| | | \textcircled{1} severe constipation (hard | diarrheaFALSE |
| | | stools in the shape of large | |
| | | beads) | |
| | | \textcircled{2}? Mild constipation (ragged | |
| | | stools that look like beads) | |
| | | \textcircled{3} Dry stools (feces with a | |
| | | cracked surface) | |
| | | \textcircled{4} (4) Moist stools (superficially | |
| | | smooth stools) | |
| | | \textcircled{5} (5) loose stools (feces that | |
| | | contain a lot of water and are | |
| | | separated into lumps) | |

For each dysbiosis-related factor, group coordination and (permutational) MANOVA were performed to calculate P-value and R². It can be understood that the lower the P-value or the higher the R2, the higher the correlation between the factors and the group coordinates. In addition, locations of samples having a positive correlation with the corresponding factors were predicted by calculating center coordinates of respective dysbiosis-related factors. The analysis data was calculated using the vegan package of the statistical analysis program R. The P-value and R2 values for developmental stage 1 are shown in Table 16, and the calculation results for directionality of metadata for developmental stage 1 are shown in Table 17. The P-value and R2 values for developmental stage 2 are shown in Table 18, and the calculation results for the directionality of metadata for developmental stage 2 are shown in Table 19.

In Tables 17 and 19 below, coord1 and coord2 are the positions of the correlation arrows on the coordinates of dysbiosis-related factors that are in significant correlation with each group. Coord1 indicates coordinate values on the horizontal axis and coord2 indicates coordinate values on the vertical axis. Arrows on the coordinate indicate corresponding directions and lengths according to extents of correlation with each group.

TABLE 16
Correlation of dysbiosis-related factors for developmental stage 1

| Metadata type | R² | P-value |
|---|---|---|
| lactation_bf | 0.040322 | 0.0599 |
| age_month | 0.070071 | 0.0918 |
| birth_mode_natural | 0.018764 | 0.2866 |
| Antibiotics | 0.007639 | 0.6059 |
| Diarrhea | 0.0049 | 0.7248 |

TABLE 17
Directionality of dysbiosis-related factors for developmental stage 1

| Metadata type | coord1 | coord2 |
|---|---|---|
| age_month | 0.017031 | −0.26416 |
| antibioticsFALSE | 0.001282 | 0.009315 |
| antibioticsTRUE | −0.004248 | −0.030855 |
| diarheaFALSE | −0.002856 | −0.000619 |
| diarheaTRUE | 0.062828 | 0.013628 |
| birth_mode_naturalFALSE | 0.020454 | 0.030504 |
| birth_mode_naturalTRUE | −0.010909 | −0.016269 |
| lactation_bfFALSE | 0.031211 | −0.016939 |
| lactation_bfTRUE | −0.038259 | 0.020764 |

TABLE 18
Correlation of dysbiosis-related factors for developmental stage 2

| Metadata type | R² | P-value |
|---|---|---|
| antibiotics | 0.075639 | 0.0211 |
| age_month | 0.118405 | 0.0482 |
| birth_mode_natural | 0.058039 | 0.0489 |
| diarrhea | 0.017417 | 0.4513 |

TABLE 19
Directionality of dysbiosis-related factors for developmental stage 2

| Metadata type | coord1 | coord2 |
|---|---|---|
| age_month | −0.097983 | 0.329856 |
| antibioticsFALSE | −0.03849 | −0.022217 |
| antibioticsTRUE | 0.070565 | 0.040732 |
| diarrheaFALSE | −0.005357 | 0.00231 |
| diarheaTRUE | 0.131238 | −0.056605 |
| birth_mode_naturalFALSE | 0.056386 | 0.028105 |
| birth_mode_naturalTRUE | −0.03947 | −0.019673 |

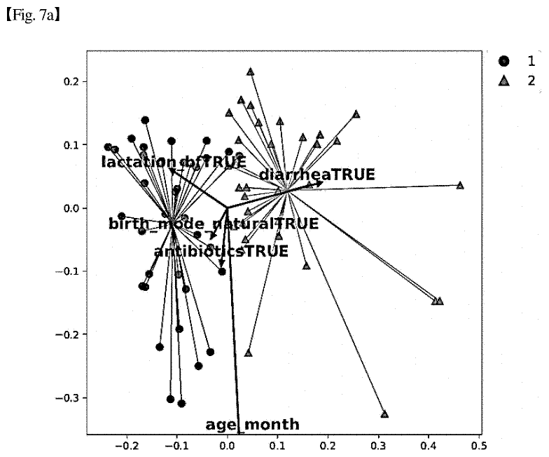

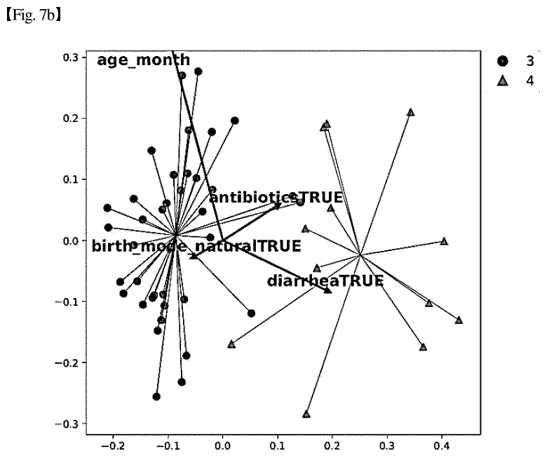

FIGS. 7A and 7B are plots showing arrows on the coordinates of dysbiosis-related factors according to each developmental stage grouped as a result of DMM grouping. Among the dysbiosis-related factors of the selected metadata, the post-birth month (age_month) is shown to have R² values of 0.070071 and 0.118405, respectively, in Tables 16 and 18, indicating higher correlation than the other dysbiosis-related factors. Referring to FIG. 7 in consideration of the coordinate values of Tables 17 and 19, the age-month can be understood to be a more discriminant factor than the other factors. This data implies that the age-month is a factor having the greatest influence on the developmental stages, but is not the reference on the basis of which the two groups in each developmental stage are divided. That is, the age-month does not have a significant influence on dysbiosis.

With reference to FIGS. 7A and 7B, the two groups developmental stage 1 (FIG. 7A) and developmental stage 2 (FIG. 7B) classified according to Example 3-2 can be understood to correlate with coordinate values for the presence or absence of diarrhea, whether or not antibiotics are taken, the modes of delivery, and whether or not breastfeeding. Referring to FIG. 7A, developmental stage 1

In the developmental stage 1 divided according to Example 3-2, the coordinate values of breastfeeding (lactation_bfTRUE) and natural delivery (birth_mode_naturalTRUE) were directed toward the center point of the group 1, compared to the coordinate values of antibiotics TRUE and diarrhea TRUE, and the center point of the group 2 shows a strong correlation with the coordinate value of diarrheaTRUE. According to panel B of FIG. 7, the coordinate values of natural delivery (birth_mode_naturalTRUE) are directed toward the center point of group 1, compared to the coordinate values of antibiotics administration (antibioticsTRUE) and diarrhea (diarrheaTRUE), and the center point of group 2 shows a stronger correlation with the coordinate values for antibiotic taking (antibioticsTRUE) and diarrhea (diarrheaTRUE).

Therefore, the two groups divided by DMM grouping in each of developmental stage 1 and developmental stage 2 show strong correlations with dysbiosis metadata as well as gut microbe analysis data. Particularly, the groups relevant to diarrhea, caesarean section, antibiotic use, and formula feeding, which are known to cause dysbiosis, can be defined as being associated with dysbiosis. In addition, within the same developmental stage, a sample group distinct from the dysbiosis-related group can be defined as a group relevant to gut balance because it is associated with breastfeeding and natural delivery. Metadata factors having strong correlations with gut microbe imbalance and balance are given in Table 20.

TABLE 20

| Developmental | Balance/ | | | |
|---|---|---|---|---|
| stage | imbalance | Related Metadata Factor | coord1 | coord2 |
| Developmental | Balance group | birth_mode_naturalTRUE | −0.010909 | −0.016269 |
| stage 1 | | lactation_bfTRUE | −0.038259 | 0.020764 |
| | Imbalance group | diarheaTRUE | 0.062828 | 0.013628 |
| | | antibioticsTRUE | −0.004248 | −0.030855 |
| Developmental | Balance group | birth_mode_naturalTRUE | −0.03947 | −0.019673 |
| stage 2 | Imbalance group | diarheaTRUE | 0.131238 | −0.056605 |
| | | antibioticsTRUE | 0.070565 | 0.040732 |

5-3. Application of Machine Learning Model According to Developmental Stage

According to Example 3-2, the gut microbiome analysis data of the gut microbe imbalance and balance group for each developmental stage, as defined above, was applied to machine learning. The regularization parameter corresponding to a hyperparameter for the model, that is, the infant dysbiosis prediction model optimized according to the present disclosure was selected by a value that allowed the best prediction result, among the 2 values of Mathematical Formula 1. The optimized prediction value for determining imbalance or balance in developmental stage 1 was determined to be 0.05 while the optimized prediction value of the developmental stage 2 was determined to be 100.

5-4. Feature Selection of Biomarker by Dysbiosis Prediction Model (Primary)

As a result of primary feature selection, biomarkers relevant to the gut balance group were found in 31 taxa at the species level and 26 taxa at the genus level. On the other hand, biomarkers relevant to the imbalance group were found in 26 taxa at the species level and 24 taxa at the genus level. In Tables 21 to 28, biomarkers relevant to balance and imbalance groups at species and genus levels are summarized according to developmental stage.

In Tables 21 to 28, the coefficient was obtained by calculating 13 of Mathematical Formula 4, and its negative and positive values mean microbes characteristic of the balance group and the imbalance group, respectively. Robustness was obtained by calculating cases where corresponding microbes appeared as relevant results during 100 bootstrap replications, and robustness closer to 1 means that the microbe is more characteristic of the corresponding group. In addition, balance group and imbalance group proportions, which mean abundance of respective microbiomes, are expressed as percentages of reads of corresponding microbes relative to a total number of reads of entire microbes.

TABLE 21
Balance group-Related Biomarker at Species Level (Developmental Stage 1)

| Sample No. (species level) | | | Balance group | Imbalance |
|---|---|---|---|---|
| (Microbe name) | coefficients | robustness | ratio (%) | group ratio (%) |
| Bifidobacterium longum | −0.070612 | 0.933333 | 51.243026 | 21.23793 |
| Lactobacillus gasseri | −0.031707 | 0.616667 | 1.055237 | 0.22722 |
| Streptococcus peroris | −0.023587 | 0.4 | 0.814519 | 0.221563 |
| Bifidobacterium bifidum | −0.011173 | 0.3 | 1.745036 | 0.563617 |
| Enterococcus faecalis | −0.011012 | 0.3 | 3.588835 | 1.564981 |
| Streptococcus pneumonia | −0.007745 | 0.166667 | 0.203145 | 0.075435 |
| Bifidobacterium breve | −0.005682 | 0.216667 | 8.686701 | 7.201174 |
| Rothia mucilaginosa | −0.001013 | 0.033333 | 0.397782 | 0.075418 |
| Streptococcus salivarius | −0.000717 | 0.016667 | 4.78868 | 2.924272 |
| Anaerostipes hadrus | −0.00063 | 0.033333 | 0.266833 | 0.03419 |
| Enterococcus faecium | −0.000245 | 0.033333 | 6.333801 | 4.757783 |
| Eggerthella lenta | −0.000058 | 0.016667 | 0.189416 | 0.074407 |

TABLE 22
Balance Group-Related Biomarker at Genus Level (Developmental Stage 1)

| Sample No. (genus level) | | | Balance group | Imbalance |
|---|---|---|---|---|
| (Microbe name) | coefficients | robustness | ratio (%) | group ratio (%) |
| Bifidobacterium | −0.168201 | 1 | 62.580789 | 33.045218 |
| Enterococcus | −0.001554 | 0.083333 | 10.041681 | 6.371829 |
| Akkermansia | −0.000945 | 0.016667 | 0.212773 | 1.174082 |
| Rothia | −0.000901 | 0.05 | 0.407708 | 0.084993 |
| Eggerthella | −0.000268 | 0.016667 | 0.202414 | 0.076959 |
| Lactobacillus | −0.000171 | 0.016667 | 2.804877 | 2.013835 |
| Ruminococcus_g5 (Family | −0.000124 | 0.016667 | 0.727894 | 3.103867 |
| Ruminococcaceae) | | | | |
| Anaerostipes | −0.000071 | 0.016667 | 0.30337 | 0.157415 |

TABLE 23
Imbalance Group-Related Biomarker at Species Level (Developmental Stage 1)

| Sample No. (species level) | | | Balance group | Imbalance |
|---|---|---|---|---|
| (Microbe name) | coefficients | robustness | ratio (%) | group ratio (%) |
| FWNZ_s (Genus Klebsiella) | 0.000031 | 0.016667 | 0.149975 | 1.899234 |
| Flavonifractor plautii | 0.000576 | 0.033333 | 0.005163 | 0.229055 |
| Streptococcus gallolyticus | 0.001891 | 0.1 | 0.243751 | 1.754617 |
| Clostridium neonatale | 0.002055 | 0.116667 | 0.010955 | 1.006057 |
| Clostridioides difficile | 0.010121 | 0.25 | 0.091256 | 0.578815 |
| Veillonella ratti | 0.017467 | 0.333333 | 0.561349 | 2.445463 |
| Escherichia coli | 0.018013 | 0.316667 | 4.687128 | 9.505538 |
| Clostridium paraputrificum | 0.021838 | 0.333333 | 0.042032 | 0.510855 |
| Bacteroides vulgatus | 0.036621 | 0.6 | 0.140871 | 5.123635 |
| Veillonella atypica | 0.063393 | 0.716667 | 0.043712 | 1.088948 |
| Veillonella dispar | 0.171471 | 1 | 0.529256 | 4.334027 |

TABLE 24
Imbalance group-Related Biomarker at Genus Level
(Developmental Stage 1)

| Sample No. | | | Balance | Imbalance |
|---|---|---|---|---|
| (genus level) | | | group | group |
| (Microbe name) | coefficients | robustness | ratio (%) | ratio (%) |
| Pseudoflavonifractor | 0.00372 | 0.116667 | 0.009344 | 0.250999 |
| Clostridioides | 0.007392 | 0.183333 | 0.116913 | 0.580252 |
| Escherichia | 0.016107 | 0.35 | 4.706873 | 9.535802 |
| Clostridium_g24 | 0.016223 | 0.333333 | 0.052884 | 0.366841 |
| (Family | | | | |
| Lachnospiraceae) | | | | |
| Clostridium | 0.027992 | 0.566667 | 0.441519 | 3.809211 |
| Bacteroides | 0.039796 | 0.65 | 2.059269 | 8.545303 |
| Veillonella | 0.365429 | 1 | 1.498936 | 9.707073 |

TABLE 25
Balance group-Related Biomarker at Species Level
(Developmental Stage 2)

| Sample No. | | | Balance | Imbalance |
|---|---|---|---|---|
| (species level) | | | group | group |
| (Microbe name) | coefficients | robustness | ratio (%) | ratio (%) |
| Fusicatenibacter | −0.113431 | 0.716667 | 2.451463 | 0.770204 |
| saccharivorans | | | | |
| Faecalibacterium | −0.067313 | 0.7 | 11.303225 | 4.18633 |
| prausnitzii | | | | |
| Blautia faecis | −0.052685 | 0.433333 | 0.815303 | 0.01675 |
| Bifidobacterium | −0.045657 | 0.483333 | 5.608016 | 2.749555 |
| catenulatum | | | | |
| Anaerostipes | −0.031165 | 0.3 | 4.493286 | 1.038971 |
| hadrus | | | | |
| Gemmiger | −0.029734 | 0.45 | 1.934029 | 0.552129 |
| formicilis | | | | |
| Eubacterium | −0.016785 | 0.166667 | 0.841646 | 0.085065 |
| eligens | | | | |
| Blautia wexlerae | −0.010245 | 0.083333 | 5.646432 | 2.316964 |
| Ruminococcus | −0.006754 | 0.083333 | 0.950139 | 0.068682 |
| bromii | | | | |
| Eubacterium | −0.006486 | 0.1 | 1.470321 | 0.388819 |
| hallii | | | | |
| Roseburia | −0.004387 | 0.15 | 1.023162 | 0.008023 |
| inulinivorans | | | | |
| Bifidobacterium | −0.002405 | 0.033333 | 0.722383 | 0.172211 |
| bifidum | | | | |
| LT907848_s | −0.00205 | 0.033333 | 1.010872 | 0.061536 |
| (Genus | | | | |
| Anaerobutyricum) | | | | |
| Bacteroides | −0.001945 | 0.116667 | 4.954876 | 5.440352 |
| fragilis | | | | |
| Roseburia | −0.000708 | 0.016667 | 0.637494 | 0.072541 |
| cecicola | | | | |
| Clostridium | −0.000548 | 0.016667 | 1.47384 | 0.790128 |
| celatum | | | | |
| PAC001046_s | −0.00027 | 0.033333 | 0.681424 | 0.000712 |
| (Family | | | | |
| Lachnospiraceae) | | | | |
| Lactobacillus | −0.000069 | 0.016667 | 0.634118 | 0.040713 |
| rogosae | | | | |
| Bacteroides | −0.00002 | 0.016667 | 2.133196 | 0.316344 |
| uniformis | | | | |

TABLE 26
Balance Group-Related Biomarker at Genus Level
(Developmental Stage 2)

| Sample No. | | | Balance | Imbalance |
|---|---|---|---|---|
| (species level) | | | group | group |
| (Microbe name) | coefficients | robustness | ratio (%) | ratio (%) |
| Ruminococcus_g2 | −0.466909 | 0.916667 | 1.860715 | 0.070953 |
| (Family | | | | |
| Ruminococcaceae) | | | | |
| Lachnospira | −0.384064 | 0.833333 | 1.875378 | 0.12984 |
| Bacteroides | −0.22437 | 0.766667 | 17.524886 | 14.548922 |
| Faecalibacterium | −0.165892 | 0.466667 | 11.503136 | 4.195657 |
| Eubacterium_g5 | −0.116977 | 0.366667 | 2.608563 | 0.480289 |
| (Family | | | | |
| Lachnospiraceae) | | | | |
| Fusicatenibacter | −0.070078 | 0.483333 | 2.514382 | 0.791564 |
| Roseburia | −0.038164 | 0.283333 | 2.255163 | 0.32842 |
| Subdoligranulum | −0.02894 | 0.183333 | 2.476985 | 0.559366 |
| Blautia | −0.016366 | 0.233333 | 8.472013 | 3.055045 |
| CCMM_g | −0.013717 | 0.166667 | 0.811336 | 0.139387 |
| (Family | | | | |
| Erysipelotrichaceae) | | | | |
| Agathobacter | −0.013303 | 0.116667 | 0.649122 | 0.123431 |
| Akkermansia | −0.009818 | 0.116667 | 1.838978 | 2.882973 |
| Anaerostipes | −0.008317 | 0.083333 | 4.771253 | 1.473928 |
| Parasutterella | −0.008229 | 0.066667 | 0.270422 | 0.176537 |
| Romboutsia | −0.005091 | 0.066667 | 0.927612 | 0.867899 |
| PAC001046_g | −0.004184 | 0.033333 | 0.694255 | 0.000712 |
| (Family | | | | |
| Lachnospiraceae) | | | | |
| Eubacterium_g23 | −0.001176 | 0.016667 | 0.422063 | 0.00009 |
| (Family | | | | |
| Ruminococcaceae) | | | | |
| Alistipes | −0.001155 | 0.016667 | 0.398863 | 1.24756 |

TABLE 27
Imbalance group-Related Biomarker at Species Level
(Developmental Stage 2)

| Sample No. | | | Balance | Imbalance |
|---|---|---|---|---|
| (genus level) | | | group | group |
| (Microbe name) | coefficients | robustness | ratio (%) | ratio (%) |
| Streptococcus | 0.000255 | 0.016667 | 2.374956 | 4.833057 |
| salivarius | | | | |
| Bacteroides dorei | 0.000329 | 0.016667 | 1.610086 | 2.637872 |
| PAC001148_s | 0.000436 | 0.016667 | 0.278625 | 0.597653 |
| (Family | | | | |
| Lachnospiraceae) | | | | |
| FWNZ_s | 0.000743 | 0.05 | 0.032624 | 2.392581 |
| (Genus Klebsiella) | | | | |
| Haemophilus | 0.001315 | 0.033333 | 0.128404 | 0.246825 |
| parainfluenzae | | | | |
| Lactobacillus | 0.001989 | 0.016667 | 0.108647 | 0.150154 |
| paracasei | | | | |
| Bifidobacterium | 0.002892 | 0.066667 | 3.680349 | 10.12144 |
| longum | | | | |
| Bacteroides ovatus | 0.002929 | 0.066667 | 1.300072 | 2.472146 |
| Lactobacillus | 0.003348 | 0.1 | 0.187065 | 0.884733 |
| fermentum | | | | |
| Clostridioides | 0.004256 | 0.066667 | 0.019651 | 0.344302 |
| difficile | | | | |
| Veillonella ratti | 0.007767 | 0.15 | 0.663291 | 3.604658 |
| Enterococcus | 0.056388 | 0.633333 | 1.198365 | 2.516732 |
| faecium | | | | |
| Veillonella dispar | 0.060976 | 0.65 | 0.153714 | 2.407966 |
| Escherichia coli | 0.080391 | 0.8 | 0.409679 | 6.41716 |
| Bifidobacterium | 0.090213 | 0.666667 | 0.358843 | 6.825921 |
| breve | | | | |

TABLE 28
Imbalance Group-Related Biomarker at Genus Level
(Developmental Stage 2)

| Sample No. | | | Balance | Imbalance |
|---|---|---|---|---|
| (genus level) | | | group | group |
| (Microbe name) | coefficients | robustness | ratio (%) | ratio (%) |
| Clostridium_g35 | 0.000291 | 0.016667 | 0.110759 | 0.221132 |
| (Family | | | | |
| Lachnospiraceae) | | | | |
| Clostridium | 0.000469 | 0.083333 | 1.860338 | 1.850075 |
| Intestinibacter | 0.000528 | 0.016667 | 0.841446 | 1.174962 |
| Bifidobacterium | 0.000938 | 0.016667 | 12.867602 | 20.307788 |
| Sutterella | 0.001188 | 0.016667 | 0.211909 | 0.104892 |
| Hungatella | 0.001625 | 0.016667 | 0.076689 | 0.191014 |
| Prevotella | 0.005983 | 0.083333 | 1.247341 | 1.583361 |
| Streptococcus | 0.007556 | 0.116667 | 2.765987 | 5.420648 |
| Citrobacter | 0.014446 | 0.116667 | 0.130479 | 0.376519 |
| Klebsiella | 0.024247 | 0.166667 | 0.032624 | 2.397416 |
| Clostridioides | 0.034329 | 0.2 | 0.01975 | 0.344425 |
| Enterococcus | 0.079289 | 0.5 | 1.223035 | 2.543823 |
| PAC001138_g | 0.105919 | 0.416667 | 0.352784 | 0.194167 |
| (Family | | | | |
| Lachnospiraceae) | | | | |
| Haemophilus | 0.157628 | 0.566667 | 0.137701 | 0.249095 |
| Lactobacillus | 0.193791 | 0.616667 | 0.864182 | 1.122363 |
| Veillonella | 0.297158 | 0.933333 | 0.895922 | 7.832423 |
| Escherichia | 0.616597 | 0.933333 | 0.41046 | 6.437183 |

5-5. Feature Selection of Microbe by Dysbiosis Prediction Model (Final)

Selection was made of final microbial biomarkers by correcting the application result of machine learning according to the selection criteria for gut microbe balance or imbalance groups, as described in Example 3-4. In brief, a total of 5 taxa, such as Akkermansia, Bacteroides fragilis, etc., which showed larger proportions of microbial taxa in the imbalance groups by developmental stage, were excluded out of the biomarkers characteristic of the balance group. In addition, a total of 3 taxa, such as Clostridium, Sutterella, and etc., which showed larger proportions of microbial taxa in the balance group, were excluded out of biomarkers characteristic of the imbalance group. In consideration of the excluded microbial taxa, characteristic biomarkers for each developmental stage are shown in Tables 29 to 32, below. The biomarkers characteristic of the balance group were found in 12 taxa at the species level and 6 taxa at the genus level for developmental stage 1 and in 18 taxa at the species level and 16 taxa at the genus level for developmental stage 2.

Biomarkers characteristic of the imbalance group, corrected by the secondary feature selection, are given in Tables 33 to 36. The biomarkers characteristic of the imbalance group were found in 11 taxa at the specific level and 7 taxa at the genus level for developmental stage 1 and in 15 taxa at the species level and 14 taxa at the genus level for developmental stage 2.

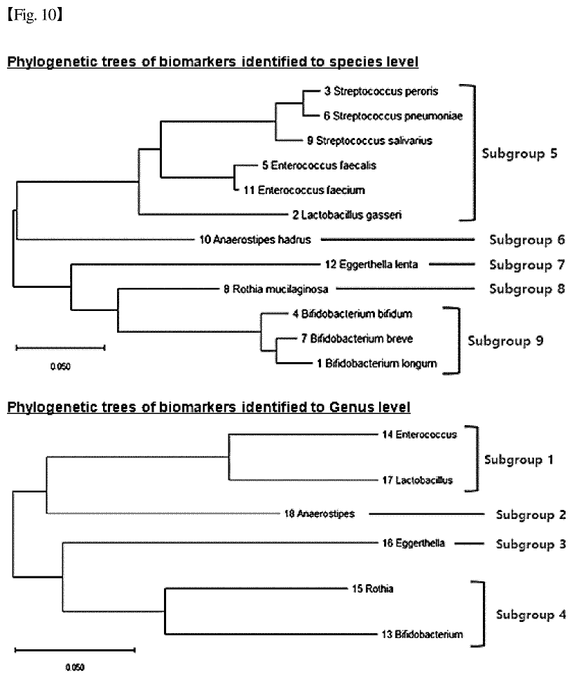

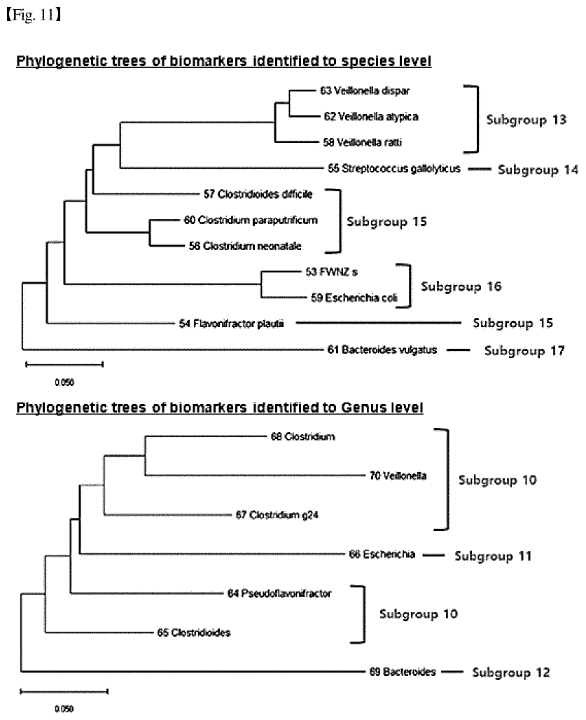

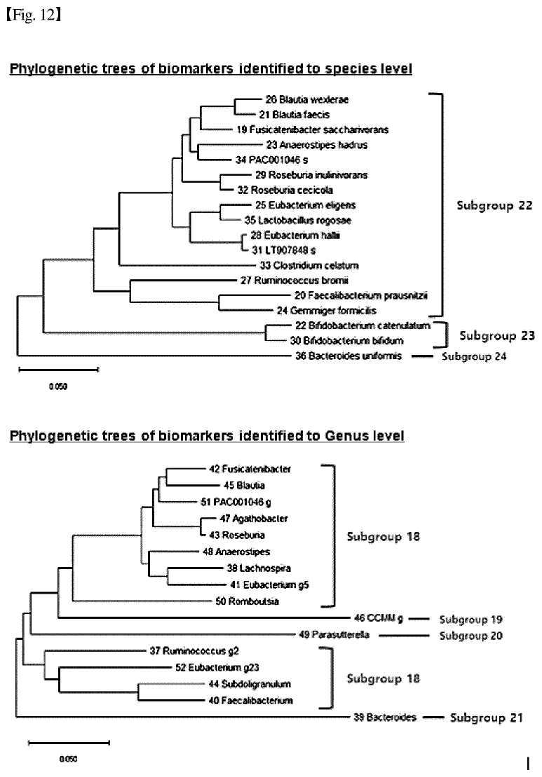

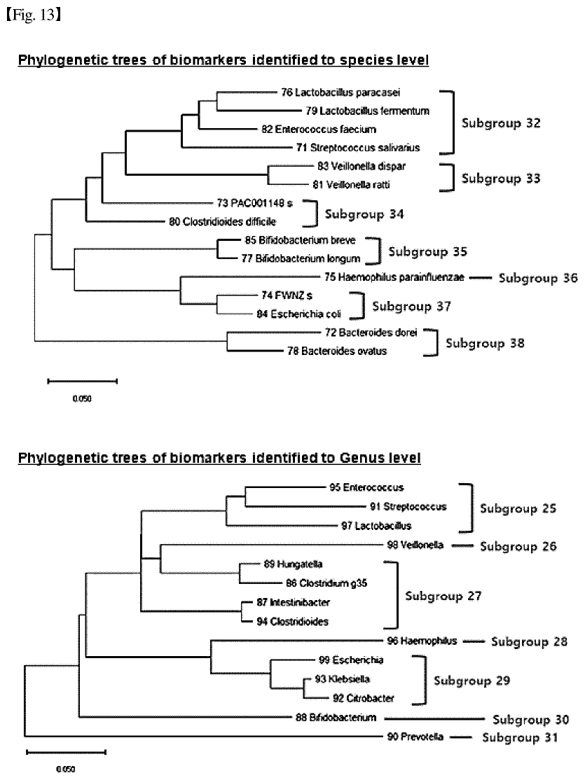

Biomarkers characteristic of the balance group for developmental stage 1 are listed in Tables 29 and 30 and depicted in the phylogenetic tree of FIG. 10, biomarkers characteristic of the imbalance group for developmental stage 1 are listed in Tables 33 and 34 and depicted in the phylogenetic tree of FIG. 11, biomarkers characteristic of the balance group for developmental stage 2 are listed in Tables 31 and 32 and depicted in the phylogenetic tree of FIG. 12, and biomarkers characteristic of the imbalance group for developmental stage 2 are listed in Tables 35 and 36 and depicted in the phylogenetic tree of FIG. 13.

In Tables 30, 32, 34, and 36, below, biomarkers at the genus level refer to microbial biomarkers which allow the discrimination of species, but are not specifically identified to the species level and thus mean microbial biomarkers actually discriminated at the species level.

TABLE 29
Balance Group-Related Biomarker at Species Level
(Developmental Stage 1)

| Sample No. | | 16S rRNA | Balance | Imbalance |
|---|---|---|---|---|
| (species level) | 16S rRNA | Fragment | group | group |
| (Microbe name) | SEQ ID NO: | SEQ ID NO: | ratio (%) | ratio (%) |
| Bifidobacterium | 1 | 81 | 51.243026 | 21.23793 |
| longum | | | | |
| Lactobacillus | 2 | 82 | 1.055237 | 0.22722 |
| gasseri | | | | |
| Streptococcus | 3 | 83 | 0.814519 | 0.221563 |
| peroris | | | | |
| Bifidobacterium | 4 | 84 | 1.745036 | 0.563617 |
| bifidum | | | | |
| Enterococcus | 5 | 85 | 3.588835 | 1.564981 |
| faecalis | | | | |
| Streptococcus | 6 | 86 | 0.203145 | 0.075435 |
| pneumoniae | | | | |
| Bifidobacterium | 7 | 87 | 8.686701 | 7.201174 |
| breve | | | | |
| Rothia | 8 | 88 | 0.397782 | 0.075418 |
| mucilaginosa | | | | |
| Streptococcus | 9 | 89 | 4.78868 | 2.924272 |
| salivarius | | | | |
| Anaerostipes | 10 | 90 | 0.266833 | 0.03419 |
| hadrus | | | | |
| Enterococcus | 11 | 91 | 6.333801 | 4.757783 |
| faecium | | | | |
| Eggerthella | 12 | 92 | 0.189416 | 0.074407 |
| lenta | | | | |

TABLE 30
Balance Group-Related Biomarker at Genus Level
(Developmental Stage 1)

| Sample No. | | 16S rRNA | Balance | Imbalance |
|---|---|---|---|---|
| (genus level) | 16S rRNA | Fragment | group | group |
| (Microbe name) | SEQ ID NO: | SEQ ID NO: | ratio (%) | ratio (%) |
| Bifidobacterium | 13 | 93 | 62.580789 | 33.045218 |
| Enterococcus | 14 | 94 | 10.041681 | 6.371829 |
| Rothia | 15 | 95 | 0.407708 | 0.084993 |
| Eggerthella | 16 | 96 | 0.202414 | 0.076959 |
| Lactobacillus | 17 | 97 | 2.804877 | 2.013835 |
| Anaerostipes | 18 | 98 | 0.30337 | 0.157415 |

TABLE 31
Balance Group-Related Biomarker at Species Level
(Developmental Stage 2)

| Sample No. | | 16S rRNA | Balance | Imbalance |
|---|---|---|---|---|
| (species level) | 16S rRNA | Fragment | group | group |
| (Microbe name) | SEQ ID NO: | SEQ ID NO: | ratio (%) | ratio (%) |
| Fusicatenibacter | 19 | 99 | 2.451463 | 0.770204 |
| saccharivorans | | | | |
| Faecalibacterium | 20 | 100 | 11.303225 | 4.18633 |
| prausnitzii | | | | |
| Blautia faecis | 21 | 101 | 0.815303 | 0.01675 |
| Bifidobacterium | 22 | 102 | 5.608016 | 2.749555 |
| catenulatum | | | | |
| Anaerostipes hadrus | 10 | 90 | 4.493286 | 1.038971 |
| Gemmiger formicilis | 23 | 103 | 1.934029 | 0.552129 |
| Eubacterium eligens | 24 | 104 | 0.841646 | 0.085065 |
| Blautia wexlerae | 25 | 105 | 5.646432 | 2.316964 |
| Ruminococcus | 26 | 106 | 0.950139 | 0.068682 |
| bromii | | | | |
| Eubacterium hallii | 27 | 107 | 1.470321 | 0.388819 |
| Roseburia | 28 | 108 | 1.023162 | 0.008023 |
| inulinivorans | | | | |
| Bifidobacterium | 4 | 84 | 0.722383 | 0.172211 |
| bifidum | | | | |
| LT907848_s | 29 | 109 | 1.010872 | 0.061536 |
| (Genus | | | | |
| Anaerobutyricum) | | | | |
| Roseburia cecicola | 30 | 110 | 0.637494 | 0.072541 |
| Clostridium celatum | 31 | ill | 1.47384 | 0.790128 |
| PAC001046_s | 32 | 112 | 0.681424 | 0.000712 |
| (Family | | | | |
| Lachnospiraceae) | | | | |
| Lactobacillus | 33 | 113 | 0.634118 | 0.040713 |
| rogosae | | | | |
| Bacteroides | 34 | 114 | 2.133196 | 0.316344 |
| uniformis | | | | |

TABLE 32
Balance Group-Related Biomarker at Genus Level
(Developmental Stage 2)

| Sample No. | | 16S rRNA | Balance | Imbalance |
|---|---|---|---|---|
| (genus level) | 16S rRNA | Fragment | group | group |
| (Microbe name) | SEQ ID NO: | SEQ ID NO: | ratio (%) | ratio (%) |
| Ruminococcus_g2 | 35 | 115 | 1.860715 | 0.070953 |
| (Family | | | | |
| Ruminococcaceae) | | | | |
| Lachnospira | 36 | 116 | 1.875378 | 0.12984 |
| Bacteroides | 37 | 117 | 17.524886 | 14.548922 |
| Faecalibacterium | 38 | 118 | 11.503136 | 4.195657 |
| Eubacterium_g5 | 39 | 119 | 2.608563 | 0.480289 |
| (Family | | | | |
| Lachnospiraceae) | | | | |
| Fusicatenibacter | 40 | 120 | 2.514382 | 0.791564 |
| Roseburia | 41 | 121 | 2.255163 | 0.32842 |
| Subdoligranulum | 42 | 122 | 2.476985 | 0.559366 |
| Blautia | 43 | 123 | 8.472013 | 3.055045 |
| CCMM_g | 44 | 124 | 0.811336 | 0.139387 |
| (Family | | | | |
| Erysipelotrichaceae) | | | | |
| Agathobacter | 45 | 125 | 0.649122 | 0.123431 |
| Anaerostipes | 18 | 98 | 4.771253 | 1.473928 |
| Parasutterella | 46 | 126 | 0.270422 | 0.176537 |
| Romboutsia | 47 | 127 | 0.927612 | 0.867899 |
| PAC001046_g | 48 | 128 | 0.694255 | 0.000712 |
| (Family | | | | |
| Lachnospiraceae) | | | | |
| Eubacterium_g23 | 49 | 129 | 0.422063 | 0.00009 |
| (Family | | | | |
| Ruminococcaceae) | | | | |

TABLE 33
Imbalance Group-Related Biomarker at Species Level
(Developmental Stage 1)

| Sample No. | | 16S rRNA | Balance | Imbalance |
|---|---|---|---|---|
| (species level) | 16S rRNA | Fragment | group | group |
| (Microbe name) | SEQ ID NO: | SEQ ID NO: | ratio (%) | ratio (%) |
| FWNZ_s | 50 | 130 | 0.149975 | 1.899234 |
| (Genus Klebsiella) | | | | |
| Flavonifractor | 51 | 131 | 0.005163 | 0.229055 |
| plautii | | | | |
| Streptococcus | 52 | 132 | 0.243751 | 1.754617 |
| gallolyticus | | | | |
| Clostridium | 53 | 133 | 0.010955 | 1.006057 |
| neonatale | | | | |
| Clostridioides | 54 | 134 | 0.091256 | 0.578815 |
| difficile | | | | |
| Veillonella ratti | 55 | 135 | 0.561349 | 2.445463 |
| Escherichia coli | 56 | 136 | 4.687128 | 9.505538 |
| Clostridium | 57 | 137 | 0.042032 | 0.510855 |
| paraputrificum | | | | |
| Bacteroides | 58 | 138 | 0.140871 | 5.123635 |
| vulgatus | | | | |
| Veillonella atypica | 59 | 139 | 0.043712 | 1.088948 |
| Veillonella dispar | 60 | 140 | 0.529256 | 4.334027 |

TABLE 34
Imbalance Group-Related Biomarker at Genus Level
(Developmental Stage 1)

| Sample No. | | 16S rRNA | Balance | Imbalance |
|---|---|---|---|---|
| (genus level) | 16S rRNA | Fragment | group | group |
| (Microbe name) | SEQ ID NO: | SEQ ID NO: | ratio (%) | ratio (%) |
| Pseudoflavonifractor | 61 | 141 | 0.009344 | 0.250999 |
| Clostridioides | 62 | 142 | 0.116913 | 0.580252 |
| Escherichia | 63 | 143 | 4.706873 | 9.535802 |
| Clostridium_g24 | 64 | 144 | 0.052884 | 0.366841 |
| (Family | | | | |
| Lachnospiraceae) | | | | |
| Clostridium | 65 | 145 | 0.441519 | 3.809211 |
| Bacteroides | 37 | 117 | 2.059269 | 8.545303 |
| Veillonella | 66 | 146 | 1.498936 | 9.707073 |

TABLE 35
Imbalance Group-Related Biomarker at Species Level
(Developmental Stage 2)

| Sample No. | | 16S rRNA | Balance | Imbalance |
|---|---|---|---|---|
| (species level) | 16S rRNA | Fragment | group | group |
| (Microbe name) | SEQ ID NO: | SEQ ID NO: | ratio (%) | ratio (%) |
| Streptococcus | 9 | 89 | 2.374956 | 4.833057 |
| salivarius | | | | |
| Bacteroides dorei | 67 | 147 | 1.610086 | 2.637872 |
| PAC01148_s | 68 | 148 | 0.278625 | 0.597653 |
| (Family | | | | |
| Lachnospiraceae) | | | | |
| FWNZ_s | 50 | 130 | 0.032624 | 2.392581 |
| (Genus Klebsiella) | | | | |
| Haemophilus | 69 | 149 | 0.128404 | 0.246825 |
| parainfluenzae | | | | |
| Lactobacillus | 70 | 150 | 0.108647 | 0.150154 |
| paracasei | | | | |
| Bifidobacterium | 1 | 81 | 3.680349 | 10.12144 |
| longum | | | | |
| Bacteroides ovatus | 71 | 151 | 1.300072 | 2.472146 |
| Lactobacillus | 72 | 152 | 0.187065 | 0.884733 |
| fermentum | | | | |
| Clostridioides | 54 | 134 | 0.019651 | 0.344302 |
| difficile | | | | |
| Veillonella ratti | 55 | 135 | 0.663291 | 3.604658 |
| Enterococcus | 11 | 91 | 1.198365 | 2.516732 |
| faecium | | | | |
| Veillonella dispar | 60 | 140 | 0.153714 | 2.407966 |
| Escherichia coli | 56 | 136 | 0.409679 | 6.41716 |
| Bifidobacterium | 7 | 87 | 0.358843 | 6.825921 |
| breve | | | | |

TABLE 36
Imbalance Group-Related Biomarker at Genus Level
(Developmental Stage 1)

| Sample No. | | 16S rRNA | Balance | Imbalance |
|---|---|---|---|---|
| (species level) | 16S rRNA | Fragment | group | group |
| (Microbe name) | SEQ ID NO: | SEQ ID NO: | ratio (%) | ratio (%) |
| Clostridium_g35 | 73 | 153 | 0.110759 | 0.221132 |
| (Family | | | | |
| Lachnospiraceae) | | | | |
| Intestinibacter | 74 | 154 | 0.841446 | 1.174962 |
| Bifidobacterium | 13 | 93 | 12.867602 | 20.307788 |
| Hungatella | 75 | 155 | 0.076689 | 0.191014 |
| Prevolella | 76 | 156 | 1.247341 | 1.583361 |
| Streptococcus | 77 | 157 | 2.765987 | 5.420648 |
| Citrobacter | 78 | 158 | 0.130479 | 0.376519 |
| Klebsiella | 79 | 159 | 0.032624 | 2.397416 |
| Clostridioides | 62 | 142 | 0.01975 | 0.344425 |
| Enterococcus | 14 | 94 | 1.223035 | 2.543823 |
| Haemophilus | 80 | 160 | 0.137701 | 0.249095 |
| Lactobacillus | 17 | 97 | 0.864182 | 1.122363 |
| Veillonella | 66 | 146 | 0.895922 | 7.832423 |
| Escherichia | 63 | 143 | 0.41046 | 6.437183 |

### Example 6: Prediction of Dysbiosis in Infant

6-1. Verification of Infant Dysbiosis Prediction Model

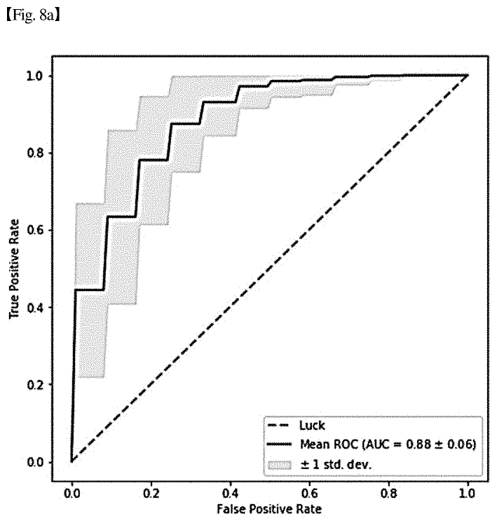

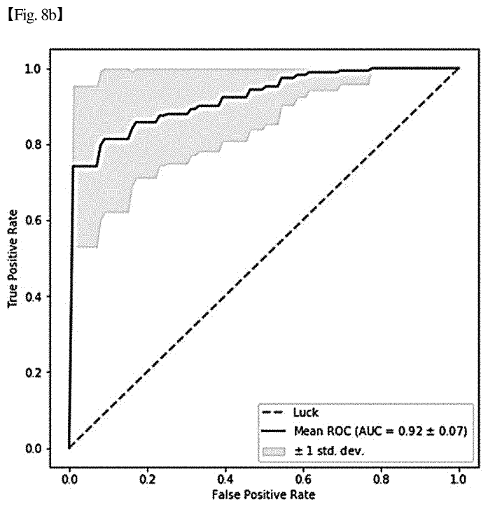

Using the method of Example 3-5, it was examined whether the machine learning model that has been trained for infant dysbiosis can actually discriminate between the presence or absence of infant dysbiosis with accuracy. As the results of determining whether or not dysbiosis exists for the test sets, ROC (receiver operating curve) and AUC (area under curve) are depicted in FIGS. 8A and 8B. The ROC is greatly bent in a bow shape and the AUC values are 0.88 for developmental stage 1 and 0.92 for developmental stage 2, which are close to 1. Thus, it can be understood that the prediction results for infant dysbiosis, obtained by the application in Example 5-3, is significant.

6-2: Determination Index of Infant Dysbiosis

In order to make an accurate clinical interpretation, as described in Example 3-6, the probability value between 0 and 1 calculated by multiplying the proportion with the coefficient of the corresponding biomarker was rescaled by dividing it by the ratio of the gut microbe imbalance and balance groups used for training. In Mathematical Formula 7, \hat{p} is a prediction score of a test subject for determining dysbiosis in infants, P₀ is a proportion of dysbiosis samples present in the training set used to construct the prediction model, N_{case} is the number of dysbiosis samples in the training set, and Nₜᵣₐᵢₙ is the total number of samples in the training set. The calculated index is named “infant dysbiosis index”.

In order to verify that the infant dysbiosis index can be used as an index for discriminating the dysbiosis state with respect to an unknown sample, sensitivity, specificity, and accuracy values were checked through the infant dysbiosis index.

Through the infant development index, the sensitivity, the specificity, and the accuracy were checked. In Mathematical Formulas 8 to 10, FP is the number of cases in which the infant development index (\hat{p}) is greater than the cut-off in the samples corresponding to developmental stage 1, FN is the number of cases in which the infant development index (\hat{p}) is smaller than the cut-off in the samples corresponding to the developmental stage 1, TP is the number of cases in which the infant development index (\hat{p}) is greater than the cut-off in the samples corresponding to developmental stage 2, and TN is the number of cases in which the infant development index (\hat{p}) is smaller than the cut-off in the samples corresponding to the developmental stage 2. These results are depicted in FIG. 6.

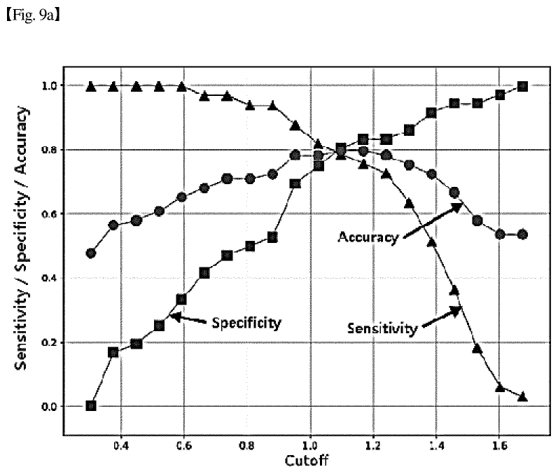

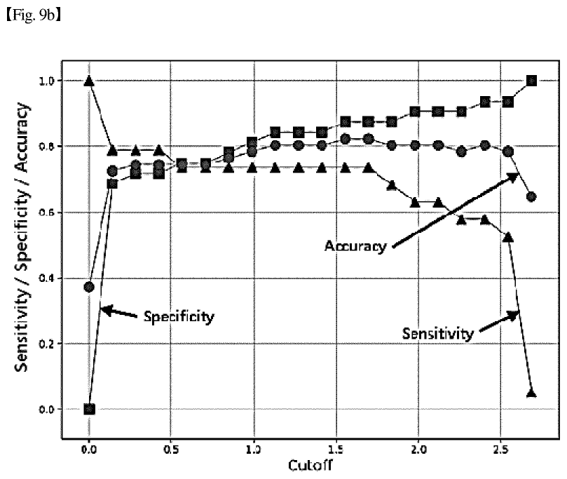

In detail, the cut-off of the infant dysbiosis index is determined by dividing the sensitivity, specificity, and accuracy values distributed from 0.3 to 1.67 in developmental stage 1 and from 0 to 2.68 in developmental stage 2 into 20 equal parts. The sensitivity, the specificity, and the accuracy are calculated as shown by Mathematical Formulas 8 to 10. In Mathematical Formulas 8 to 10, TP is the number of cases in which the infant dysbiosis index (\hat{p}) is greater than the cut-off in the gut microbe imbalance samples, TN is the number of cases in which the infant dysbiosis index (\hat{p}) is smaller than the cut-off in the gut microbe imbalance samples, FP is the number of cases in which the infant dysbiosis index (\hat{p}) is greater than the cut-off in the gut balance samples, and FN is the number of cases in which the infant dysbiosis index (\hat{p}) is smaller than the cut-off in the gut microbe balance samples. These calculation results are depicted in FIGS. 9A and 9B.

When dysbiosis states were determined on the basis of the highest index 1.17 at which accuracy was calculated to be 80% for developmental stage 1, the specificity which indicates the probability of infant gut microbe balance was 83% and the sensitivity which indicates the probability of infant microbe imbalance was 76%. The accuracy plot is given in FIG. 9A.

When dysbiosis states were determined on the basis of the infant development index of 1.7 at which accuracy was calculated to be 82% for developmental stage 2, the specificity which indicates the probability of infant gut microbe balance was 88% and the sensitivity which indicates the probability of infant microbe imbalance was 74%. The corresponding accuracy plot is given in FIG. 9B.

When dysbiosis is measured on the basis of the infant development index of 1.17 for developmental stage 1 and 1.7 for developmental stage 2, the specificity was measured to be about 83% and 88%, respectively, and as such, can accurately determine whether or the gut microbiome is in a balanced state, demonstrating its high clinical discriminating potential.

Therefore, when the infant dysbiosis index of a sample to be tested is 1.19 or greater for developmental stage 1, the infant can be determined to be in a gut microbe imbalance state. When the infant dysbiosis index is calculated to be below 1.17, the infant can be determined to be in a gut microbe balance state. When the infant dysbiosis index of a sample to be tested is 1.19 or greater for developmental stage 2, the infant can be determined to be in a gut microbe imbalance state. When the infant dysbiosis index is calculated to be below 1.17, the infant can be determined to be in a gut microbe balance state.

6-3. Determination of Infant Dysbiosis

The presence or absence of dysbiosis by developmental stage of infants can be determined by analyzing biomarkers characteristic of infant dysbiosis. Since the infant dysbiosis biomarkers vary depending on infant developmental stages, the determination of infant developmental stages should precede the determination of infant dysbiosis.

After gut microbiomes are analyzed using the method of Example 2 and infant developmental stages are determined using the method of Example 4, the data thus obtained are applied to the infant dysbiosis prediction model to obtain an infant dysbiosis determination index. According to dysbiosis determination index criteria by developmental stage, determination can be finally made of dysbiosis in the infant.

In detail, the species of microbial biomarkers characteristic of the balance or unbalance group for each developmental stage of infants and the proportion (relative abundance) of these species in the gut microflora are analyzed to calculate dysbiosis determination indices, and cut-off values of the dysbiosis determination indices are set using the accuracy, sensitivity, and specificity. A balance group is given to the case where the index is below the cut-off value and an imbalance group is given to the case wherein the index is as high as or higher than the cut-off value. Exemplary criteria for determining infant dysbiosis are listed in Table 37, below.

TABLE 37
Criteria for Determining Infant Dysbiosis

| Developmental | Infant Dysbiosis | |
|---|---|---|
| stage | Prediction Model | Dysbiosis |
| Developmental | Dysbiosis discrimination | Gut microbe balance |
| stage 1 | index less than 1.17 | |
| | Dysbiosis discrimination | Gut microbe imbalance |
| | index 1.17 or higher | |
| Developmental | Dysbiosis discrimination | Gut microbe balance |
| stage 2 | index less than 1.17 | |
| | Dysbiosis discrimination | Gut microbe imbalance |
| | index 1.17 or higher | |

For adults who have a relatively stable gut microbial ecosystem, a difference in the distribution of gut microbiome is evident between healthy and diseased groups. In adults, dysbiosis refers to a condition in which the gut microbiome becomes less diverse and out of balance due to factors such as antibiotics, processed foods, etc. Recently, dysbiosis has been pointed out as a factor in modem diseases such as irritable bowel syndrome (IBS), obesity, diabetes, and the like.

In the case of the gut microbial ecosystem of infants, microbes settle in the intestines of germ-free newborns, and this process may appear somewhat unstable as the species diversity is analyzed at a lower level compared to adults. Therefore, determination of dysbiosis in infants requires investigation characteristics of the metadata for populations collected in the same developmental periods, with the species diversity as the criterion for determining dysbiosis being excluded.

In order to determine the infant dysbiosis, as stated above, it is important to find out what kind of metadata has the greatest impact on gut microbiome analysis data. Therefore, the present inventors selected items such as delivery mode, lactation type, whether or not antibiotics are taken, and diarrhea, which are metadata relevant to infant dysbiosis, through literature surveys, and investigated which items have the greatest influence on gut microbial data grouped by developmental stage. Subsequently, biomarkers were selected by searching for microbial species that are given meaning and strongly correlate with each other in each group through metadata.

6-4. Correlation with Infant Gut Microbial Biomarker

Phylogenetic trees of infant dysbiosis biomarkers were built using a neighbor joining algorithm on the basis of 16S rRNA sequences of biomarkers characteristic of infant gut microbe imbalance and balance groups, with subgroups divided from the point of view of taxonomic classification (order level). The biomarkers appearing in infant gut imbalance and balance groups for each developmental stage can be divided into 38 subgroups.

FIGS. 10 to 13 depict phylogenetic trees of species- and genus-level biomarkers characteristic of infant gut microbe imbalance and balance groups by developmental stage. In detail, classifications of developmental stages, balance/imbalance, and species/genus-level markers are summarized in the following table.

TABLE 38

| Developmental | Balance/ | | |
|---|---|---|---|
| stage | Imbalance | Species/Genus (Marker) | Subgroup |
| Developmental | Balance | Characteristic genus-level | Groups 1 to 4 |
| stage 1 | group | marker | |
| | | Characteristic species-level | Groups 5 to 9 |
| | | marker | |
| | Imbalance | Characteristic genus-level | Groups 10 to 12 |
| | group | marker | |
| | | Characteristic species-level | Groups 13 to 17 |
| | | marker | |
| Developmental | Balance | Characteristic genus-level | Groups 18 to 21 |
| stage 2 | group | marker | |
| | | Characteristic species-level | Groups 22 to 24 |
| | | marker | |
| | Imbalance | Characteristic genus-level | Groups 25 to 31 |
| | group | marker | |
| | | Characteristic species-level | Groups 32 to 38 |
| | | marker | |

(A) Biomarker of Gut Balance Group in Developmental Stage 1

With reference to FIG. 10 in which biomarkers of the infant gut balance group in developmental stage 1 are depicted, the biomarker that exhibits the greatest correlation at the genus level with gut microbe balance group in developmental stage 1 of infants is Bifidobacterium, which includes lactic acid bacteria and belongs to the Actinobacteria phylum, together with Rothia in the same subgroup. Lactobacillus and Enterococcus in subgroup 1 are also lactic acid bacteria, indicating that the more positive influences of lactic acid bacteria, such as immunopotentiation, nutrient absorption, etc., appear in the balance group in developmental stage 1.

In greater detail, among Lactobacillus, Lactobacillus gasseri (Subgroup 5), which is most characteristic of the balance group in terms of coefficient value, can survive bile acids like most Lactobacillus species, and can settle in the large intestine for a long period of time as the bacteria has a gene that easily attaches to intestinal epithelial cells. In addition, effects such as strengthening immunity and relieving intestinal discomfort, diarrhea, and constipation have all been verified through clinical studies on adults. As the bacterium which is most characteristic of the balance group in terms of coefficient value, Bifidobacterium longum (subgroup 9) is a lactic acid bacterium detected in the mother's vagina, is associated with natural delivery, and helps the immunity of the newborn. In addition, many adult studies have reported cases of alleviating inflammatory bowel disease (IBD) such as Crohn's disease (CD) or ulcerative colitis (UC). As such, biomarkers characteristic of the gut balance group in development stage 1 have a prominent positive role in the development of intestinal microflora in infants.

(B) Biomarker of Gut Imbalance Group in Developmental Stage 1

Referring to FIG. 11 which shows biomarkers for the infant gut imbalance group in developmental stage 1, biomarkers which are the most characteristic of the infant gut microbe imbalance group in developmental stage 1 belong to the Veillonella and Clostridium genera of the Firmicutes phylum and to the Bacteroides genus of the Bacteroidetes phylum.

These microbes are involved in the metabolism of solid (baby and general) diets consisting of vegetable carbohydrates (simple sugars and fibers) and proteins, not liquid (feeding) or gel (weaning) dietary forms, and are characterized by producing short chain fatty acids. For adults, microbes with the ability to metabolize fiber and produce short-chain fatty acids play a beneficial role in increasing the species diversity of the gut microbial ecosystem. For infants, however, it was analyzed that the detection pattern of these microbes in developmental stage 1 was associated with dysbiosis factors such as diarrhea, cesarean section, etc. The detection of microbes appearing at the time of a diet change to solid types (baby and general foods) in developmental stage 1 where infants mainly consume liquid (feeding) to gel (weaning) diets can be interpreted as an earlier-than-expected development of the intestinal microbial ecosystem, indicating that the gut microbial ecosystem does not develop ideally from the point of view of factors associated with dysbiosis.

(C) Biomarker of Gut Balance Group in Developmental Stage 2

Referring to FIG. 12 which shows biomarkers for the infant gut balance group in developmental stage 1, biomarkers which exhibit the greatest correlation with the gut microbe imbalance group in developmental stage 2 of infants are, for the most part, short-chain fatty acid producing bacteria. Among 16 biomarkers at the genus level, the 13 biomarkers in subgroup 18 (Subdoligranulum, Faecalibacterium, Eubacterium_g23, Ruminococcus_g2, Romboutsia, Fusicatenibacter, Anaerostipes, Agathobacter, Lachnospira, Roseburia, Eubacterium_g5, Blautia, and PAC001046_g) belong to the Firmicutes phylum and are representative short-chain fatty acid-producing microbes in a taxonomic level below the Clostridiales order of the Firmicutes phylum.

Short-chain fatty acids, which are metabolites produced through degradation of dietary fiber, are known to have beneficial effects on the human body, such as promotion of energy production and vitamin production, and reinforcement of colonocyte association. The process that the gut microbial ecosystem properly develops with the change of dietary forms from liquid type (feeding) to gel type (weaning) to solid type (baby and general foods) is grounds for the determination. Particularly, as a representative short-chain fatty acid producing bacterium, Faecalibacterium prausnitzii, which exhibits relatively high coefficient and robustness values, has been reported to have anti-inflammatory effect in adults. Eubacterium eligens, Anaerostipes hadrus, and Blautia wexlerae are also short-chain fatty acid producing bacteria and typically produce butyric acid, which modulates immunity and relieves inflammation. Eubacterium eligens helps digestion by decomposing the water-soluble dietary fiber pectin that is abundantly present in fruits and vegetables. It is known that Anaerostipes hadrus is associated with the alleviation of irritable bowel syndrome and Blautia wexlerae is present in the intestines of obese people at a lower rate than in healthy people.

(D) Biomarker of Gut Imbalance Group in Developmental Stage 2

Referring to FIG. 13 which shows biomarkers of the gut imbalance group in developmental stage 2, the most characteristic biomarkers are the lactic acid bacteria Enterococcus, Lactobacillus, Streptococcus (subgroup 25), and Bifidobacterium (subgroup 30). In addition, Haemophilus (subgroup 28), Escherichia, Klebsiella, and Citrobacter (subgroup 29), which are enteric bacteria in the Proteobacteria phylum, were also selected as biomarkers associated with developmental stage 2. Enteric bacteria are microbes that help to stabilize the initial gut environment, but they have a detrimental effect in an unbalanced gut environment. Haemophilus can cause inflammation in the intestine. In particular, Clostridioides difficile is the causative agent of difficile infection.

The four genera (Clostridium_g35, Hungatella, Clostridioides, and Intestinibacter) in the Clostridiales order (Subgroup 27), which produce short-chain fatty acids, are included as biomarkers characteristic of the infant gut imbalance group. However, since the robustness value of the biomarker is lower than that of the microbial species in the Clostridiales order, which are biomarkers characteristic of the gut microbe balance group, it can be seen that there are relatively few cases in which the biomarker is significantly calculated.

Turning to Prevotella and Bacteroides of the Bacteroidetes phylum at developmental stage 2, Bacteroides is included in biomarkers characteristic of the balance group while Prevotella is included in biomarkers characteristic of the imbalance group. Thus, the more ideal type of gut microbial ecosystem during infancy is considered to be the Bacteroides type.

Therefore, the detection of a biomarker characteristic of the infant gut microbe imbalance group in developmental stage 2 can be interpreted as a state in which the initial intestinal microbial ecosystem is still maintained and is developing later than expected. As the result is related to the dysbiosis factor, the gut microbial ecosystem is not ideally developed.

1. A method for detecting a developmental stage of gut microbiota and a degree of gut microbiota dysbiosis in infant, the method comprising the steps of:
(A) obtaining gut microbiome information of microbial types discriminated at a species level and abundance ratios of the microbial types for gut microbiome in a test infant;
(B) obtaining metadata information of the test infant;
(C) determining a developmental stage of the gut microbiome according to criteria for classifying developmental stage of a reference infant, on the basis of at least one selected from the group consisting of the gut microbiome information of step (A) and the metadata information of step (B); and
(D) determining the degree of the gut microbiota dysbiosis according to the determined developmental stage, by using biomarkers characteristic of imbalance group and biomarkers characteristic of balance group in each developmental stage.

2. The method of claim 1, wherein the step (A) of obtaining gut microbiome information comprise the steps of:
(A-1) obtaining genomic DNA of gut microbes from a fecal specimen of the test infant;
(A-2) obtaining 16S rRNA genetic information from the genomic DNA of the gut microbes; and
(A-3) analyzing the gut microbiome information of microbial types discriminated at a species level and abundance ratios of the microbial types for gut microbiome in the test infant, by performing an analysis of the 16S rRNA information of gut microbes.

3. The method of claim 1, wherein the criteria for classifying developmental stage of a reference infant are obtained by performing the steps comprising:
(A′) obtaining gut microbiome information of microbial types discriminated at a species level and abundance ratios of the microbial types for gut microbiome in the reference infant;
(B′) obtaining metadata information of the reference infant; and
(C) determining criteria for classifying developmental stage of the reference infant, on the basis of at least one selected from the group consisting of the gut microbiome information of step (A′) and the metadata information of step (B′).

4. The method of claim 1, wherein the metadata information of the test infant comprise at least one factor selected from the group consisting of sex, months of age, height, weight, diet type, feeding mode, feeding of lactic acid bacterium-containing diet, fecal type, fecal color, information on antibiotic use, and information on diagnosed diseases of the infant, and mother's diet type during a gestation period, and mother's diet type and antibiotic administration after delivery.

5. The method of claim 1, wherein the step (C) of determining a developmental stage is conducted using at least one selected from the group consisting of dietary step of the test infant, months of age of the test infant, and microbial biomarkers characteristic of developmental stages.

6. The method of claim 3, wherein the step (C′) of determining criteria for classifying developmental stage of the reference infant is conducted using at least one selected from the group consisting of dietary step of the reference infant, months of age of the reference infant, and microbial biomarkers characteristic of developmental stages.

7. The method of claim 5, wherein when developmental stage is determined to developmental stage 1 or developmental stage 2 by using microbial biomarkers characteristic of developmental stage, the biomarker characteristic of developmental stage 1 is at least one selected from the group consisting of microbes listed in Tables 10 and 11, and the biomarker characteristic of developmental stage 2 is at least one selected from the group consisting of microbes listed in Tables 12 and 13.

8. The method of claim 3, wherein the step (C′) of determining criteria for classifying developmental stage of the reference infant is conducted by calculating an infant development index with analyzing microbial types discriminated at a species level and abundance ratios of the microbial types for gut microbiome, for microbial biomarkers characteristic of developmental stages 1 and 2 and setting a cut-off value for the infant development index with using accuracy, sensitivity and specificity; and imparting developmental stage 1 to a case where the development index is less than the cut-off value, and imparting developmental stage 2 to a case where the development index is equal to and higher than the cut-off value.

9. The method of claim 8, wherein the step (C) of determining a developmental stage is conducted by calculating the infant development index with analyzing microbial types of microbial biomarkers characteristic of developmental stage of the test infant and abundance ratios of the microbial types, and
imparting developmental stage 1 where the calculated infant development index of the test infant is less than the cut-off value of reference infant, and imparting developmental stage 2 where the development index is equal to and higher than the cut-off value.

10. The method of claim 8, wherein the infant development index is calculated using the following Mathematical Formulas 4 to 7:
$$\begin{matrix}p = \frac{1}{1 + {e}^{- β \cdot X}} = {logit}^{- 1} ( β \cdot X ) = {logit}^{- 1} ( {β}_{0} + {\sum }_{j = 1}^{m} {β}_{j} {x}_{j} ) & [ Mathematical Formula 4 ] \\ {min}_{β} λ {‖ β ‖}_{1} + {\sum }_{i = 1}^{n} log ( {e}^{- {y}_{i} ( β \cdot {X}_{i} )} + 1 ) & [ Mathematical Formula 5 ] \\ \hat{p} = {logit}^{- 1} ( \hat{β} \cdot {X}^{′} ) = \frac{1}{1 + {e}^{- \hat{β} \cdot {X}^{′}}} & [ Mathematical Formula 6 ] \\ Infant development index = \frac{\hat{p}}{{p}_{o}} = \frac{\hat{p}}{{N}_{case} / {N}_{train}} & [ Mathematical Formula 7 ]\end{matrix}$$

11. The method of claim 5, wherein the developmental stages are classified in terms of dietary steps including liquid-phase, gel-phase, and solid-phase diets.

12. The method of claim 5, wherein the criteria for classifying developmental stages of a reference infant in the step (C) of determining a developmental stage include whether solid-phase diet is fed or not, whether the infant is older than 15 months or not, and whether the infant development index meets 1.19 or not.

13. The method of claim 1, wherein the biomarker characteristic of the balance group for each developmental stage in step (D) is at least one selected from the group consisting of the microbes listed in Tables 29 to 32 and the biomarker characteristic of the imbalance group for each developmental stage is at least one selected from the group consisting of the microbes listed in Tables 33 to 36.

14. The method of claim 3, wherein the method may further comprise step (D′) of selecting criteria for determining imbalance group in each developmental stage after the step (C′) of determining criteria for classifying developmental stages of the reference infant, and
in step (D′), calculating an imbalance determination index with analyzing microbial types of microbial biomarkers characteristic of imbalance group and microbial biomarkers characteristic of balance group in each developmental stage, and abundance ratios of the microbial types and setting a cut-off value for the imbalance determination index with using accuracy, sensitivity and specificity; and imparting balance group to a case where the development index is less than the cut-off value, and imparting imbalance group to a case where the development index is equal to and higher than the cut-off value.

15. The method of claim 14, wherein the step (D) of determining an imbalance group or a balance group of gut microbiome is conducted by calculating the imbalance determination index with analyzing microbial types of microbial biomarkers characteristic of imbalance group and balance group in each developmental stage of the test infant and proportions (abundance ratios) of the microbial types, and
imparting balance group to a case where the calculated imbalance determination index of the test infant is less than the cut-off value of reference infant, and imparting imbalance group to a case where the calculated imbalance determination index is equal to and higher than the cut-off value.
determining species of microbial biomarkers characteristic of balance and imbalance groups of each developmental stage in the reference infant, analyzing a proportion (abundance ratio) of the species in gut microflora to calculate an imbalance determination index, and determining the test infant as a balance group when the calculated imbalance determination index of the test infant is less than a cut-off value set as a reference criterion for the imbalance determination index of a reference infant and as an imbalance group when the calculated imbalance determination of the test infant is as high as or higher than the cut-off value.

16. The method of claim 11, wherein the imbalance determination index is calculated using the following Mathematical Formulas 4 to 7:
$$\begin{matrix}p = \frac{1}{1 + {e}^{- β \cdot X}} = {logit}^{- 1} ( β \cdot X ) = {logit}^{- 1} ( {β}_{0} + {\sum }_{j = 1}^{m} {β}_{j} {x}_{j} ) & [ Mathematical Formula 4 ] \\ {min}_{β} λ {‖ β ‖}_{1} + {\sum }_{i = 1}^{n} log ( {e}^{- {y}_{i} ( β \cdot {X}_{i} )} + 1 ) & [ Mathematical Formula 5 ] \\ \hat{p} = {logit}^{- 1} ( \hat{β} \cdot {X}^{′} ) = \frac{1}{1 + {e}^{- \hat{β} \cdot {X}^{′}}} & [ Mathematical Formula 6 ] \\ Infant dysbiosis index = \frac{\hat{p}}{{p}_{o}} = \frac{\hat{p}}{{N}_{case} / {N}_{train}} & [ Mathematical Formula 7 ]\end{matrix}$$

17. The method of claim 1, further comprising a step of monitoring a change of imbalance determination index in the test infant with time, after the step of (D).

18. The method of claim 1, further comprising a step of achieving a gut microbial balance by conducing at least one measure selected from the group consisting of probiotics, probiotics, medication, diets, and life habits on the basis of the developmental stage of gut microbiota and the degree of gut microbiota dysbiosis in the test infant.

19. The method of claim 18, wherein the probiotics include at least one microbial biomarker characteristic of the balance group of developmental stage 1 as shown in Tables 29 and 30, when the test infant is determined to be in developmental stage 1, and at least one microbial biomarker characteristic of the balance group of developmental 2 as shown in Tables 31 and 32, when the test infant is determined to be in developmental 2, in case that the developmental stages of the test infant are divided into developmental stage 1 and developmental stage 2.

20. The method of claim 18, wherein
in case that the developmental stages of the test infant are divided into developmental stage 1 and developmental stage 2,
when the test infant is determined to be in developmental stage 1, the prebiotics include a material increasing a relative abundance of at least one of the microbial biomarkers characteristic of the balance group of developmental stage 1 as listed in Tables 29 and 30, or a material decreasing a relative abundance of at least one of the microbial biomarkers characteristic of the imbalance group of developmental stage 1 as listed in Tables 33 and 34, or
when the test infant is determined to be under developmental stage 2, the prebiotics include a material increasing a relative abundance (relative abundance ratio) of at least one of the microbial biomarkers characteristic of the balance group of developmental stage 2 as listed in Tables 31 and 32, or a material decreasing a relative abundance of at least one of the microbial biomarkers characteristic of the imbalance group of developmental stage 2 as listed in Tables 35 and 36.

21. The method of claim 1, wherein, in step (D),
the balance group of developmental stage 1 is a group in which a biomarker characteristic of the balance group influenced by natural delivery and breastfeeding is detected,
the imbalance group of developmental stage 1 is a group in which a biomarker characteristic of the imbalance group influenced by diarrhea and antibiotic administration,
the balance group of developmental stage 2 is a group in which a biomarker characteristic of the balance group influenced by natural delivery is detected, and
the imbalance group of developmental stage 2 is a group in which a biomarker characteristic of the imbalance group influenced by diarrhea and antibiotic administration.


SEQUENCE LISTING (162 sequences)

SEQ ID NO: 1 - DNA, 1447 bp, Artificial Sequence
  Full 16sRNA of Bifidobacterium longum group
gatgaacgctggcggcgtgcttaacacatgcaagtcgaacgggatccatcaagcttgcttggtggtgagagtggcgaacgggtgagtaatgcgtgaccgacctgccccatacaccggaatagctcctggaaacgggtggtaatgccggatgttccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgagagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacggtcgtagagatacggcttcccttcggggcgggttcacaggtggtgcatggtcgtcgtcagctcgtgtcgtgagatgttgggttaagtcccgcaacgagcgcaaccctcgccccgtgttgccagcggattatgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgtgggatggagccgtctaaggtgaggctcgtgattgggact

SEQ ID NO: 2 - DNA, 1495 bp, Artificial Sequence
  Full 16sRNA of Lactobacillus gasseri group
gacgaacgctggcggcgtgcctaatacatgcaagtcgagcgagcttgcctagatgaatttggtgcttgcaccagatgaaactagatacaagcgagcggcggacgggtgagtaacacgtgggtaacctgcccaagagactgggataacacctggaaacagatgctaataccggataacaacactagacgcatgtctagagtttaaaagatggttctgctatcactcttggatggacctgcggtgcattagctagttggtaaggtaacggcttaccaaggcaatgatgcatagccgagttgagagactgatcggccacattgggactgagacacggcccaaactcctacgggaggcagcagtagggaatcttccacaatggacgcaagtctgatggagcaacgccgcgtgagtgaagaagggtttcggctcgtaaagctctgttggtagtgaagaaagatagaggtagtaactggcctttatttgacggtaattacttagaaagtcacggctaactacgtgccagcagccgcggtaatacgtaggtggcaagcgttgtccggatttattgggcgtaaagcgagtgcaggcggttcaataagtctgatgtgaaagccttcggctcaaccggagaattgcatcagaaactgttgaacttgagtgcagaagaggagagtggaactccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatgagtgctaagtgttgggaggtttccgcctctcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccagtgcaaacctaagagattaggtgttcccttcggggacgctgagacaggtggtgcatggctgtcgtcagctcgtgtcgtgagatgttgggttaagtcccgcaacgagcgcaacccttgtcattagttgccatcattaagttgggcactctaatgagactgccggtgacaaaccggaggaaggtggggatgacgtcaagtcatcatgccccttatgacctgggctacacacgtgctacaatggacggtacaacgagaagcgaacctgcgaaggcaagcggatctctgaaagccgttctcagttcggactgtaggctgcaactcgcctacacgaagctggaatcgctagtaatcgcggatcagcacgccgcggtgaatacgttcccgggccttgtacacaccgcccgtcacaccatgagagtctgtaacacccaaagccggtgggataacctttataggagtcagccgtctaaggtaggacagatgattagggtg

SEQ ID NO: 3 - DNA, 1468 bp, Artificial Sequence
  Full 16sRNA of Streptococcus peroris group
gatgaacgctggcggcgtgcctaatacatgcaagtagaacgctgaaggaggagcttgcttctctggatgagttgcgaacgggtgagtaacgcgtaggtaacctgcctggtagcgggggataactattggaaacgatagctaataccgcataagagcagttgttgcatgacagctgtttaaaaggtgcaattgcaccactaccagatggacctgcgttgtattagctagttggtgaggtaacggctcaccaaggcgacgatacatagccgacctgagagggtgatcggccacactgggactgagacacggcccagactcctacgggaggcagcagtagggaatcttcggcaatgggggcaaccctgaccgagcaacgccgcgtgagtgaagaaggttttcggatcgtaaagctctgttgtaagagaagaacgagtgtgagagtggaaagttcacgctgtgacggtatcttaccagaaagggacggctaactacgtgccagcagccgcggtaatacgtaggtcccgagcgttatccggatttattgggcgtaaagcgagcgcaggcggttagataagtctgaagttaaaggctgtggcttaaccatagtacgctttggaaactgtttaacttgagtgcaagaggggagagtggaattccatgtgtagcggtgaaatgcgtagatatatggaggaacaccggtggcgaaagcggctctctggcttgtaactgacgctgaggctcgaaagcgtggggagcaaacaggattagataccctggtagtccacgccgtaaacgatgagtgctaggtgttagaccctttccggggtttagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcccgatgccatttctagagataggaagttacttcggtacatcggtgacaggtggtgcatggttgtcgtcagctcgtgtcgtgagatgttgggttaagtcccgcaacgagcgcaacccctattgttagttgccatcattcagttgggcactctagcgagactgccggtaataaaccggaggaaggtggggatgacgtcaaatcatcatgccccttatgacctgggctacacacgtgctacaatggctggtacaacgagtcgcgagtcggtgacggcaagctaatctcttaaagccagtctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccatttggagccagccgcctaaggtgggatagatgattggggtg

SEQ ID NO: 4 - DNA, 1450 bp, Artificial Sequence
  Full 16sRNA of Bifidobacterium bifidum
gatgaacgctggcggcgtgcttaacacatgcaagtcgaacgggatccatcaagcttgcttggtggtgagagtggcgaacgggtgagtaatgcgtgaccgacctgccccatgctccggaatagctcctggaaacgggtggtaatgccggatgttccacatgatcgcatgtgattgtgggaaagattctatcggcgtgggatggggtcgcgtcctatcagcttgttggtgaggtaacggctcaccaaggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttgtttgggagcaagccttcgggtgagtgtacctttcgaataagcgccggctaactacgtgccagcagccgcggtaatacgtagggcgcaagcgttatccggatttattgggcgtaaagggctcgtaggcggctcgtcgcgtccggtgtgaaagtccatcgcttaacggtggatctgcgccgggtacgggcgggctggagtgcggtaggggagactggaattcccggtgtaacggtggaatgtgtagatatcgggaagaacaccgatggcgaaggcaggtctctgggccgtcactgacgctgaggagcgaaagcgtggggagcgaacaggattagataccctggtagtccacgccgtaaacggtggacgctggatgtggggcacgttccacgtgttccgtgtcggagctaacgcgttaagcgtcccgcctggggagtacggccgcaaggctaaaactcaaagaaattgacgggggcccgcacaagcggcggagcatgcggattaattcgatgcaacgcgaagaaccttacctgggcttgacatgttcccgacgacgccagagatggcgtttcccttcggggcgggttcacaggtggtgcatggtcgtcgtcagctcgtgtcgtgagatgttgggttaagtcccgcaacgagcgcaaccctcgccccgtgttgccagcacgttatggtgggaactcacgggggaccgccggggttaactcggaggaaggtggggatgacgtcagatcatcatgccccttacgtccagggcttcacgcatgctacaatggccggtacagcgggatgcgacatggcgacatggagcggatccctgaaaaccggtctcagttcggatcggagcctgcaacccggctccgtgaaggcggagtcgctagtaatcgcggatcagcaacgccgcggtgaatgcgttcccgggccttgtacacaccgcccgtcaagtcatgaaagtgggcagcacccgaagccggtggcctaaccccttgtgggatggagccgtctaaggtgaggctcgtgattgggact

SEQ ID NO: 5 - DNA, 1483 bp, Artificial Sequence
  Full 16sRNA of Enterococcus faecalis
gacgaacgctggcggcgtgcctaatacatgcaagtcgaacgcttctttcctcccgagtgcttgcactcaattggaaagaggagtggcggacgggtgagtaacacgtgggtaacctacccatcagagggggataacacttggaaacaggtgctaataccgcataacagtttatgccgcatggcataagagtgaaaggcgctttcgggtgtcgctgatggatggacccgcggtgcattagctagttggtgaggtaacggctcaccaaggccacgatgcatagccgacctgagagggtgatcggccacactgggactgagacacggcccagactcctacgggaggcagcagtagggaatcttcggcaatggacgaaagtctgaccgagcaacgccgcgtgagtgaagaaggttttcggatcgtaaaactctgttgttagagaagaacaaggacgttagtaactgaacgtcccctgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagcaaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagctttcccttcggggacaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgctagaccgcgaggtcatgcaaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagagtttgtaacacccgaagtcggtgaggtaacctttttggagccagccgcctaaggtgggatagatgattggggtg

SEQ ID NO: 6 - DNA, 1468 bp, Artificial Sequence
  Full 16sRNA of Streptococcus pneumoniae group
gacgaacgctggcggcgtgcctaatacatgcaagtagaacgctgaaggaggagcttgcttctctggatgagttgcgaacgggtgagtaacgcgtaggtaacctgcctggtagcgggggataactattggaaacgatagctaataccgcataagagtagatgttgcatgacatttgcttaaaaggtgcacttgcatcactaccagatggacctgcgttgtattagctagttggtggggtaacggctcaccaaggcgacgatacatagccgacctgagagggtgatcggccacactgggactgagacacggcccagactcctacgggaggcagcagtagggaatcttcggcaatggacggaagtctgaccgagcaacgccgcgtgagtgaagaaggttttcggatcgtaaagctctgttgtaagagaagaacgagtgtgagagtggaaagttcacactgtgacggtatcttaccagaaagggacggctaactacgtgccagcagccgcggtaatacgtaggtcccgagcgttgtccggatttattgggcgtaaagcgagcgcaggcggttagataagtctgaagttaaaggctgtggcttaaccatagtaggctttggaaactgtttaacttgagtgcaagaggggagagtggaattccatgtgtagcggtgaaatgcgtagatatatggaggaacaccggtggcgaaagcggctctctggcttgtaactgacgctgaggctcgaaagcgtggggagcaaacaggattagataccctggtagtccacgctgtaaacgatgagtgctaggtgttagaccctttccggggtttagtgccgtagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccctctgacgactctagagatagagttttccttcgggacagaggtgacaggtggtgcatggttgtcgtcagctcgtgtcgtgagatgttgggttaagtcccgcaacgagcgcaacccctattgttagttgccatcatttagttgggcactctagcgagactgccggtaataaaccggaggaaggtggggatgacgtcaaatcatcatgccccttatgacctgggctacacacgtgctacaatggctggtacaacgagtcgcaagccggtgacggcaagctaatctcttaaagccagtctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccgtaaggagccagccgcctaaggtgggatagatgattggggtg

SEQ ID NO: 7 - DNA, 1453 bp, Artificial Sequence
  Full 16sRNA of Bifidobacterium breve
gatgaacgctggcggcgtgcttaacacatgcaagtcgaacgggatccatcgggctttgcttggtggtgagagtggcgaacgggtgagtaatgcgtgaccgacctgccccatgcaccggaatagctcctggaaacgggtggtaatgccggatgctccatcacaccgcatggtgtgttgggaaagcctttgcggcatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttgttagggagcaaggcactttgtgttgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacgatcccagagatggggtttcccttcggggcgggttcacaggtggtgcatggtcgtcgtcagctcgtgtcgtgagatgttgggttaagtcccgcaacgagcgcaaccctcgccccgtgttgccagcggattgtgccgggaactcacgggggaccgccggggttaactcggaggaaggtggggatgacgtcagatcatcatgccccttacgtccagggcttcacgcatgctacaatggccggtacaacgggatgcgacagtgcgagctggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgcgggagggagccgtctaaggtgaggctcgtgattgggact

SEQ ID NO: 8 - DNA, 1448 bp, Artificial Sequence
  Full 16sRNA of Rothia mucilaginosa group
gacgaacgctggcggcgtgcttaacacatgcaagtcgaacgatgaagcctagcttgctaggtggattagtggcgaacgggtgagtaatacgtgagtaacctacctttaactctgggataagcctgggaaactgggtctaataccggatacgaccaatctccgcatggggtgttggtggaaagcgttatgtagtggttatagatgggctcacggcctatcagcttgttggtgaggtaacggctcaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctgttagcagggaagaagagaaattgacggtacctgcagagaaagcgccggctaactacgtgccagcagccgcggtaatacgtagggcgcgagcgttgtccggaattattgggcgtaaagagcttgtaggcggtttgtcgcgtctgctgtgaaaggccggagcttaactccggtattgcagtgggtacgggcagactagagtgcagtaggggagactggaactcctggtgtagcggtggaatgcgcagatatcaggaagaacaccgatggcgaaggcaggtctctgggctgtaactgacgctgagaagcgaaagcatggggagcgaacaggattagataccctggtagtccatgccgtaaacgttgggcactaggtgtgggggacattccacgttttccgcgccgtagctaacgcattaagtgccccgcctggggagtacggccgcaaggctaaaactcaaagaaattgacgggggcccgcacaagcggcggagcatgcggattaattcgatgcaacgcgaagaaccttaccaaggcttgacatatactggaccgcatcagagatggtgtttcccttcggggctggtatacaggtggtgcatggttgtcgtcagctcgtgtcgtgagatgttgggttaagtcccgcaacgagcgcaaccctcgttctatgttgccagcacgtaatggtggggactcataggagactgccggggtcaactcggaggaaggtggggatgacgtcaaatcatcatgccccttatgtcttgggcttcacgcatgctacaatggccggtacagagggttgcgatactgtgaggtggagctaatccctaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagttggtaacacccaaagccggtggcctaaccttttggagggagccgtctaaggtgggattggcgattgggact

SEQ ID NO: 9 - DNA, 1470 bp, Artificial Sequence
  Full 16sRNA of Streptococcus salivarius group
gacgaacgctggcggcgtgcctaatacatgcaagtagaacgctgaagagaggagcttgctcttcttggatgagttgcgaacgggtgagtaacgcgtaggtaacctgccttgtagcgggggataactattggaaacgatagctaataccgcataacaatggatgacacatgtcatttatttgaaaggggcaattgctccactacaagatggacctgcgttgtattagctagtaggtgaggtaacggctcacctaggcgacgatacatagccgacctgagagggtgatcggccacactgggactgagacacggcccagactcctacgggaggcagcagtagggaatcttcggcaatgggggcaaccctgaccgagcaacgccgcgtgagtgaagaaggttttcggatcgtaaagctctgttgtaagtcaagaacgagtgtgagagtggaaagttcacactgtgacggtagcttaccagaaagggacggctaactacgtgccagcagccgcggtaatacgtaggtcccgagcgttgtccggatttattgggcgtaaagcgagcgcaggcggtttgataagtctgaagttaaaggctgtggctcaaccatagttcgctttggaaactgtcaaacttgagtgcagaaggggagagtggaattccatgtgtagcggtgaaatgcgtagatatatggaggaacaccggtggcgaaagcggctctctggtctgtaactgacgctgaggctcgaaagcgtggggagcgaacaggattagataccctggtagtccacgccgtaaacgatgagtgctaggtgttggatcctttccgggattcagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcccgatgctatttctagagatagaaagttacttcggtacatcggtgacaggtggtgcatggttgtcgtcagctcgtgtcgtgagatgttgggttaagtcccgcaacgagcgcaacccctattgttagttgccatcattcagttgggcactctagcgagactgccggtaataaaccggaggaaggtggggatgacgtcaaatcatcatgccccttatgacctgggctacacacgtgctacaatggttggtacaacgagttgcgagtcggtgacggcaagctaatctcttaaagccaatctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggatagatgattggggtg

SEQ ID NO: 10 - DNA, 1455 bp, Artificial Sequence
  Full 16sRNA of Anaerostipes hadrus group
gatgaacgctggcggcgtgcttaacacatgcaagtcgaacgaaacaccttatttgattttcttcggaactgaagatttggtgattgagtggcggacgggtgagtaacgcgtgggtaacctgccctgtacagggggataacagtcagaaatgactgctaataccgcataagaccacagcaccgcatggtgcaggggtaaaaactccggtggtacaggatggacccgcgtctgattagctggttggtgaggtaacggctcaccaaggcgacgatcagtagccggcttgagagagtgaacggccacattgggactgagacacggcccaaactcctacgggaggcagcagtggggaatattgcacaatgggggaaaccctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggaattactgggtgtaaagggtgcgtaggtggtatggcaagtcagaagtgaaaacccagggcttaactctgggactgcttttgaaactgtcagactggagtgcaggagaggtaagcggaattcctagtgtagcggtgaaatgcgtagatattaggaggaacatcagtggcgaaggcggcttactggactgaaactgacactgaggcacgaaagcgtggggagcaaacaggattagataccctggtagtccacgccgtaaacgatgaatactaggtgtcggggccgtaggggcttcggtgccgcagccaacgcagtaagtattccacctggggagtacgttcgcaagaatgaaactcaaaggaattgacggggacccgcacaagcggtggagcatgtggtttaattcgaagcaacgcgaagaaccttacctggtcttgacatccttctgaccggtccttaaccggacctttccttcgggacaggagagacaggtggtgcatggttgtcgtcagctcgtgtcgtgagatgttgggttaagtcccgcaacgagcgcaacccctatctttagtagccagcatataaggtgggcactctagagagactgccagggataacctggaggaaggtggggacgacgtcaaatcatcatgccccttatgaccagggctacacacgtgctacaatggcgtaaacagagggaagcagcctcgtgagagtgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgtaaggagggagctgccgaaggcgggaccgataactggggtg

SEQ ID NO: 11 - DNA, 1482 bp, Artificial Sequence
  Full 16sRNA of Enterococcus faecium group
gacgaacgctggcggcgtgcctaatacatgcaagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatgacctgggctacacacgtgctacaatgggaagtacaacgagttgcgaagtcgcgaggctaagctaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggatagatgattggggtg

SEQ ID NO: 12 - DNA, 1431 bp, Artificial Sequence
  Full 16sRNA of Eggerthella lenta
gatgaacgctggcggcgtgcctaacacatgcaagtcgaacgatgaaaccgccctcgggcggacatgaagtggcgaacgggtgagtaacacgtgaccaacctgccccttgctccgggacaaccttgggaaaccgaggctaataccggatactcctcgcccccctcctggggggcccgggaaagcccagacggcaagggatggggtcgcggcccattaggtagtaggcggggtaacggcccacctagcccgcgatgggtagccgggttgagagaccgaccggccacattgggactgagatacggcccagactcctacgggaggcagcagtggggaattttgcgcaatgggggaaaccctgacgcagcaacgccgcgtgcgggacgacggccttcgggttgtaaaccgctttcagcagggaagaaattcgacggtacctgcagaagaagctccggctaactacgtgccagcagccgcggtaatacgtagggagcgagcgttatccggattcattgggcgtaaagagcgcgtaggcggcctctcaagcgggatctctaatccgagggctcaacccccggccggatcccgaactgggaggctcgagttcggtagaggcaggcggaattcccggtgtagcggtggaatgcgcagatatcgggaagaacaccgatggcgaaggcagcctgctgggccgcaactgacgctgaggcgcgaaagctaggggagcgaacaggattagataccctggtagtcctagccgtaaacgatggatactaggtgtggggggctccgccctccgtgccgcagccaacgcattaagtatcccgcctggggagtacggccgcaaggctaaaactcaaaggaattgacgggggcccgcacaagcagcggagcatgtggcttaattcgaagcaacgcgaagaaccttaccagggcttgacatggacgtgaagccggggaaacccggtggccgagaggagcgtccgcaggtggtgcatggctgtcgtcagctcgtgtcgtgagatgttgggttaagtcccgcaacgagcgcaacccctgccccatgttgccagcattaggttggggactcatgggggactgccggcgtcaagccggaggaaggtggggacgacgtcaagtcatcatgccctttatgccctgggctgcacacgtgctacaatggccggtacaacgggctgcgagaccgcgaggtcgagcgaatccctcaaagccggccccagttcggatcggaggctgcaacccgcctccgtgaagtcggagttgctagtaatcgcggatcagcatgccgcggtgaatacgttcccgggccttgtacacaccgcccgtcacaccacccgagtcgtctgcacccgaagccgccggccgaacccgcaaggggcggaggcgtcgaaggtgtggagggtaaggggggtg

SEQ ID NO: 13 - DNA, 1450 bp, Artificial Sequence; identical to SEQ ID NO: 4
  Full 16sRNA of Bifidobacterium

SEQ ID NO: 14 - DNA, 1483 bp, Artificial Sequence; identical to SEQ ID NO: 5
  Full 16sRNA of Enterococcus

SEQ ID NO: 15 - DNA, 1449 bp, Artificial Sequence
  Full 16sRNA of Rothia
gacgaacgctggcggcgtgcttaacacatgcaagtcgaacgatgaagcctagcttgctaggtggattagtggcgaacgggtgagtaatacgtgagtgacctacctttgactctgggataagcctgggaaactgggtctaataccggatacgaccaatctccgcatggggtgttggtggaaagcgttatggagtggttttagatgggctcacggcctatcagcttgttggtgaggtaatggcttaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctgttagcatcgaagaagcgaaagtgacggtaggtgcagagaaagcgccggctaactacgtgccagcagccgcggtaatacgtagggcgcgagcgttgtccggaattattgggcgtaaagagcttgtaggcggttggtcgcgtctgctgtgaaaggctggggcttaaccctggttttgcagtgggtacgggctaactagagtgcagtaggggagactggaattcctggtgtagcggtggaatgcgcagatatcaggaggaacaccgatggcgaaggcaggtctctgggctgtaactgacgctgagaagcgaaagcatggggagcgaacaggattagataccctggtagtccatgccgtaaacgttgggcactaggtgtgggggacattccacgttttccgcgccgtagctaacgcattaagtgccccgcctggggagtacggccgcaaggctaaaactcaaagaaattgacgggggcccgcacaagcggcggagcatgcggattaattcgatgcaacgcgaagaaccttaccaaggcttgacatatactggactgcgtcagagatggcgtttcccttcggggctggtatacaggtggtgcatggttgtcgtcagctcgtgtcgtgagatgttgggttaagtcccgcaacgagcgcaaccctcgttctatgttgccagcacgtgatggtggggactcataggagactgccggggtcaactcggaggaaggtggggatgacgtcaaatcatcatgccccttatgtcttgggcttcacgcatgctacaatggccggtacagagggttgcgatactgtgaggtggagctaatccctaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagttggtaacacccgaagccggtggcctaaccctggtggggggagccgtcgaaggtgggactggcgattgggact

SEQ ID NO: 16 - DNA, 1431 bp, Artificial Sequence; identical to SEQ ID NO: 12
  Full 16sRNA of Eggerthella

SEQ ID NO: 17 - DNA, 1485 bp, Artificial Sequence
  Full 16sRNA of Lactobacillus
gacgaacgctggcggcgtgcctaatacatgcaagtcgagcgagctgaattcaaagattccttcgggatgatttgttggacgctagcggcggatgggtgagtaacacgtgggcaatctgccctaaagactgggataccacttggaaacaggtgctaataccggataacaacatgaatcgcatgattcaagtttgaaaggcggcgtaagctgtcactttaggatgagcccgcggcgcattagctagttggtggggtaagggcctaccaaggcaatgatgcgtagccgagttgagagactgatcggccacattgggactgagacacggcccaaactcctacgggaggcagcagtagggaatcttccacaatggacgcaagtctgatggagcaacgccgcgtgagtgaagaaggttttcggatcgtaaagctctgttgttggtgaagaaggataggggcagtaactggtctttatttgacggtaatcaaccagaaagtcacggctaactacgtgccagcagccgcggtaatacgtaggtggcaagcgttgtccggatttattgggcgtaaagcgagcgcaggcggaatgataagtctgatgtgaaagcccacggctcaaccgtggaactgcatcggaaactgtcattcttgagtgcagaagaggagagtggaactccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatgagcgctaggtgttggggactttccggtcctcagtgccgcagcaaacgcattaagcgctccgcctggggagtacgaccgcaaggttgaaactcaaaggaattgacgggggcccgcacaagcggtggagcatgtggtttaattcgaagcaacgcgaagaaccttaccaggtcttgacatcctgcgcaacagctagagataggtggttcccttcggggacgcagagacaggtggtgcatggctgtcgtcagctcgtgtcgtgagatgttgggttaagtcccgcaacgagcgcaacccttgtctttagttgccatcattaagttgggcactctagagagactgccggtgacaaaccggaggaaggtggggatgacgtcaagtcatcatgccccttatgacctgggctacacacgtgctacaatgggcagtacaacgagaagcgaacccgcgagggtaagcggatctcttaaagctgttctcagttcggactgcaggctgcaactcgcctgcacgaagctggaatcgctagtaatcgcggatcagcacgccgcggtgaatacgttcccgggccttgtacacaccgcccgtcacaccatggaagtctgcaatgcccaaagtcggtgggataaccttttaggagtcagccgcctaaggcagggcagatgactggggtg

SEQ ID NO: 18 - DNA, 1456 bp, Artificial Sequence
  Full 16sRNA of Anaerostipes
gatgaacgctggcggcgtgcttaacacatgcaagtcgaacgaagcatttaggattgaagttttcggatggatttcctatatgactgagtggcggacgggtgagtaacgcgtggggaacctgccctatacagggggataacagctggaaacggctgctaataccgcataagcgcacagaatcgcatgattcagtgtgaaaagccctggcagtataggatggtcccgcgtctgattagctggttggtgaggtaacggctcaccaaggcgacgatcagtagccggcttgagagagtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaaacagacggtacctgactaagaagccccggctaactacgtgccagcagccgcggtaatacgtagggggcaagcgttatccggaattactgggtgtaaagggtgcgtaggtggcatggtaagtcagaagtgaaagcccggggcttaaccccgggactgcttttgaaactgtcatgctggagtgcaggagaggtaagcggaattcctagtgtagcggtgaaatgcgtagatattaggaggaacaccagtggcgaaggcggcttactggactgtcactgacactgatgcacgaaagcgtggggagcaaacaggattagataccctggtagtccacgccgtaaacgatgaatactaggtgtcggggccgtagaggcttcggtgccgcagcaaacgcagtaagtattccacctggggagtacgttcgcaagaatgaaactcaaaggaattgacggggacccgcacaagcggtggagcatgtggtttaattcgaagcaacgcgaagaaccttacctggtcttgacatcccaatgaccgaaccttaaccggttttttctttcgagacattggagacaggtggtgcatggttgtcgtcagctcgtgtcgtgagatgttgggttaagtcccgcaacgagcgcaacccctatctttagtagccagcatttaaggtgggcactctagagagactgccagggataacctggaggaaggtggggacgacgtcaaatcatcatgccccttatggccagggctacacacgtgctacaatggcgtaaacaaagggaagcgaagtcgtgaggcgaagcaaatcccagaaataacgtctcagttcggattgtagtctgcaactcgactacatgaagctggaatcgctagtaatcgtgaatcagaatgtcacggtgaatacgttcccgggtcttgtacacaccgcccgtcacaccatgggagtcagtaacgcccgaagtcagtgacccaaccgcaaggagggagctgccgaaggtgggaccgataactggggtg

SEQ ID NO: 19 - DNA, 1415 bp, Artificial Sequence
  Full 16sRNA of Fusicatenibacter saccharivorans
gatgaacgctggcggcgtgcttaacacatgcaagtcgagcgaagcagttaagaagattyttcggatgattcttgactgactgagcggcggacgggtgagtaacgcgtgggtgacctgccccataccgggggataacagctggaaacggctgctaataccgcataagcgcacagagctgcatggctcggtgtgaaaaactccggtggtatgggatgggcccgcgtctgattaggcagttggcggggtaacggcccaccaaaccgacgatcagtagccggcctgagagggcgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagataatgacggtacctgactaagaagccccggctaactacgtgccagcagccgcggtaatacgtagggggcaagcgttatccggatttactgggtgtaaagggagcgtagacggcaaggcaagtctgatgtgaaaacccagggcttaaccctgggactgcattggaaactgtctggctcgagtgccggagaggtaagcggaattcctagtgtagcggtgaaatgcgtagatattaggaagaacaccagtggcgaaggcggcttactggacggtaactgacgttgaggctcgaaagcgtggggagcaaacaggattagataccctggtagtccacgccgtaaacgatgaatgctaggtgttggggagcaaagctcttcggtgccgccgcaaacgcattaagcattccacctggggagtacgttcgcaagaatgaaactcaaaggaattgacggggacccgcacaagcggtggagcatgtggtttaattcgaagcaacgcgaagaaccttaccaggtcttgacatcccgatgaccggcccgtaacggggccttctcttcggagcattggagacaggtggtgcatggttgtcgtcagctcgtgtcgtgagatgttgggttaagtcccgcaacgagcgcaacccttatcctcagtagccagcaggtaaagctgggcactctgtggagactgccagggataacctggaggaaggtggggatgacgtcaaatcatcatgccccttatgatctgggctacacacgtgctacaatggcgtaaacaaagggaggcaaagccgcgaggtggagcaaatcccaaaaataacgtctcagttcggactgcagtctgcaactcgactgcacgaagctggaatcgctagtaatcgcgaatcagaatgtcgcggtgaatacgttcccgggtcttgtacacaccgcccgtcacaccatgggagttggtaacgcccgaagtcagtgacccaacctttta

SEQ ID NO: 20 - DNA, 1431 bp, Artificial Sequence
  Full 16sRNA of Faecalibacterium prausnitzii group
cgaacgctggcggcgcgcctaacacatgcaagtcgaacgagcgagagagagcttgctttctcaagcgagtggcgaacgggtgagtaacgcgtgaggaacctgcctcaaagagggggacaacagttggaaacgactgctaataccgcataagcccacgacccggcatcgggtagagggaaaaggagcaatccgctttgagatggcctcgcgtccgattagctagttggtgaggtaacggcccaccaaggcgacgatcggtagccggactgagaggttgaacggccacattgggactgagacacggcccagactcctacgggaggcagcagtggggaatattgcacaatgggggaaaccctgatgcagcgacgccgcgtggaggaagaaggtcttcggattgtaaactcctgttgttgaggaagataatgacggtactcaacaaggaagtgacggctaactacgtgccagcagccgcggtaaaacgtaggtcacaagcgttgtccggaattactgggtgtaaagggagcgcaggcgggaaggcaagttggaagtgaaatccatgggctcaacccatgaactgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagtgtgggtagcaaacaggattagataccctggtagtccacactgtggccgatgtttactaggtgttggaggattgaccccttcagtgccgcagttaacacaataagtaatccacctggggagtacgaccgcaaggttgaaactcaaaggaattgacgggggcccgcacaagcagtggagtatgtggtttaattcgacgcaacgcgaagaaccttaccaagtcttgacatcctgcgacgcacatagaaatatgtgtttccttcgggacgcagagacaggtggtgcatggttgtcgtcagctcgtgtcgtgagatgttgggttaagtcccgcaacgagcgcaacccttatggtcagttactacgcaagaggactctggccagactgccgttgacaaaacggaggaaggtggggatgacgtcaaatcatcatgccctttatgacttgggctacacacgtactacaatggcgttaaacaaagagaagcaagaccgcgaggtggagcaaaactcagaaacaacgtcccagttcggactgcaggctgcaactcgcctgcacgaagtcggaattgctagtaatcgcagatcagcatgctgcggtgaatacgttcccgggccttgtacacaccgcccgtcacaccatgagagccggggggacccgaagtcggtagtctaaccgcaaggaggacgccgccgaaggtaaaactggtgattggggtg

SEQ ID NO: 21 - DNA, 1453 bp, Artificial Sequence
  Full 16sRNA of Blautia faecis
gatgaacgctggcggcgtgcttaacacatgcaagtcgaacgggaaacattttattgaagcttcggcagatttagcttgtttctagtggcggacgggtgagtaacgcgtgggtaacctgccttataccgggggataacagccggaaatgactgctaataccgcataagcgcacagaaccgcatggttcggtgtgaaaaactccggtggtataagatggacccgcgttggattagctagttggcagggcagcggcctaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagataatgacggtacctgactaagaagccccggctaactacgtgccagcagccgcggtaatacgtagggggcaagcgttatccggatttactgggtgtaaagggagcgtagacggcgcagcaagtctgatgtgaaaggcaggggcttaacccctggactgcattggaaactgctgtgcttgagtgccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcagggagcacagctctttggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaaatcttgacatccctctgaccgggacttaaccgtccctttccttcgggacaggggagacaggtggtgcatggttgtcgtcagctcgtgtcgtgagatgttgggttaagtcccgcaacgagcgcaacccctatccttagtagccagcacgcagtggtgggcactctgaggagactgccagggataacctggaggaaggcggggatgacgtcaaatcatcatgccccttatgatttgggctacacacgtgctacaatggcgtaaacaaagggaagcgaacccgcgagggtgggcaaatctcaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaaccgcaagggaggagctgccgaaggcgggaccgatgactggggtg

SEQ ID NO: 22 - DNA, 1450 bp, Artificial Sequence
  Full 16sRNA of Bifidobacterium catenulatum group
gatgaacgctggcggcgtgcttaacacatgcaagtcgaacgggatccaggcagcttgctgcctggtgagagtggcgaacgggtgagtaatgcgtgaccgacctgccccatacaccggaatagctcctggaaacgggtggtaatgccggatgctccgactcctcgcatggggtgtcgggaaagatttcatcggtatgggatggggtcgcgtcctatcaggtagtcggcggggtaacggcccaccgagcctacgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgcttttgatcgggagcaagccttcgggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatctgcgccgggtacgggcgggctggagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccgtagagatacggtctcccttcggggcgggttcacaggtggtgcatggtcgtcgtcagctcgtgtcgtgagatgttgggttaagtcccgcaacgagcgcaaccctcgccctgtgttgccagcacgtcatggtgggaactcacgggggaccgccggggtcaactcggaggaaggtggggatgacgtcagatcatcatgccccttacgtccagggcttcacgcatgctacaatggccggtacaacgggatgcgacatggcgacatggagcggatccctgaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcctaaccccttgtgggatggagccgtctaaggtgagactcgtgattgggact

SEQ ID NO: 23 - DNA, 1406 bp, Artificial Sequence
  Full 16sRNA of Gemmiger formicilis group
catgcagtcgacggagctagaggagcttgcttttcttggcttagtggcgaacgggtgagtaacgcgtgagtaacctgccctggagtgggggacaacagttggaaacgactgctaataccgcataagcccacgatccggcatcggatcgagggaaaaggattttttcgcttcaggatggactcgcgtccaattagctagttggtgaggtaacggcccaccaaggcgacgattggtagccggactgagaggttgaacggccacattgggactgagacacggcccagactcctacgggaggcagcagtgggggatattgcacaatgggggaaaccctgatgcagcgacgccgcgtggaggaagaaggttttcggattgtaaactcctgtcgttagggacgataatgacggtacctaacaagaaagcaccggctaactacgtgccagcagccgcggtaaaacgtagggtgcaagcgttgtccggaattactgggtgtaaagggagcgcaggcggaccggcaagttggaagtgaaaactatgggctcaacccataaattgctttcaaaactgctggccttgagtagtgcagaggtaggtggaattcccggtgtagcggtggaatgcgtagatatcgggaggaacaccagtggcgaaggcgacctactgggcaccaactgacgctgaggctcgaaagcatgggtagcaaacaggattagataccctggtagtccatgccgtaaacgatgattactaggtgttggaggattgaccccttcagtgccgcagttaacacaataagtaatccacctggggagtacgaccgcaaggttgaaactcaaaggaattgacgggggcccgcacaagcagtggagtatgtggtttaattcgaagcaacgcgaagaaccttaccaggtcttgacatccgatgcatagcgcagagatgcatgaagtccttcgggacatcgagacaggtggtgcatggttgtcgtcagctcgtgtcgtgagatgttgggttaagtcccgcaacgagcgcaacccttattgccagttactacgcaagaggactctggcgagactgccgttgacaaaacggaggaaggtggggatgacgtcaaatcatcatgccctttatgacctgggctacacacgtactacaatggcgtttaacaaagagaagcaagaccgcgaggtggagcaaaactcaaaaacaacgtctcagttcagattgcaggctgcaactcgcctgcatgaagtcggaattgctagtaatcgcggatcagcatgccgcggtgaatacgttcccgggccttgtacacaccgcccgtcacaccatgagagccggggggacccgaagtcggtagtctaaccgcaaggaggacgccgccgaagtaaaactggtgattggggtg

SEQ ID NO: 24 - DNA, 1453 bp, Artificial Sequence
  Full 16sRNA of Eubacterium eligens group
gatgaacgctggcggcgtgcttaacacatgcaagtcgaacgaagcatttgcgacagattttttcggaatgaagttgcttatgactgagtggcggacgggtgagtaacgcgtgggtaacctgccttgtactgggggatagcagctggaaacggctggtaataccgcataagcgcacaatgttgcatgacatggtgtgaaaaactccggtggtataagatggacccgcgtctgattagctagttggtgagataacagcccaccaaggcgacgatcagtagccgacctgagagggtgaccggccacattgggactgagacacggcccagactcctacgggaggcagcagtggggaatattgcacaatggaggaaactctgatgcagcgacgccgcgtgagtgaagaagtaattcgttatgtaaagctctatcagcagggaagatagtgacggtacctgactaagaagctccggctaaatacgtgccagcagccgcggtaatacgtatggagcaagcgttatccggatttactgggtgtaaagggagtgtaggtggccatgcaagtcagaagtgaaaatccggggctcaaccccggaactgcttttgaaactgtaaggctggagtgcaggaggggtgagtggaattcctagtgtagcggtgaaatgcgtagatattaggaggaacaccagtggcgaaggcggctcactggactgtaactgacactgaggctcgaaagcgtggggagcaaacaggattagataccctggtagtccacgccgtaaacgatgaatactaggtgtcggggcccataagggcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccactgaccggacagtaatgtgtcctttccttcgggacagtggagacaggtggtgcatggttgtcgtcagctcgtgtcgtgagatgttgggttaagtcccgcaacgagcgcaacccctatccttagtagccagcagtaagatgggcactctagggagactgccagggataacctggaggaaggtggggatgacgtcaaatcatcatgccccttatgacttgggctacacacgtgctacaatggcgtaaacaaagtgaagcgaagtcgtgaggccaagcaaatcacaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcgaaaatgcccgaagtcggtgacctaacgtaagaaggagccgccgaaggcaggtttgataactggggtg

SEQ ID NO: 25 - DNA, 1452 bp, Artificial Sequence
  Full 16sRNA of Blautia wexlerae
gatgaacgctggcggcgtgcttaacacatgcaagtcgaacgggaattactttattgaaacttcggtcgatttaatttaattctagtggcggacgggtgagtaacgcgtgggtaacctgccttatacagggggataacagtcagaaatggctgctaataccgcataagcgcacagagctgcatggctcagtgtgaaaaactccggtggtataagatggacccgcgttggattagctagttggtggggtaacggcccaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggtgtggcaagtctgatgtgaaaggcatgggctcaacctgtggactgcattggaaactgtcatacttgagtgccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcggggagcatggctcttcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccgcctgaccgatccttaaccggatctttccttcgggacaggcgagacaggtggtgcatggttgtcgtcagctcgtgtcgtgagatgttgggttaagtcccgcaacgagcgcaacccctatcctcagtagccagcatttaaggtgggcactctggggagactgccagggataacctggaggaaggcggggatgacgtcaaatcatcatgccccttatgatttgggctacacacgtgctacaatggcgtaaacaaagggaagcgagattgtgagatggagcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagtaacgcccgaagtcagtgacctaactgcaaagaaggagctgccgaaggcgggaccgatgactggggtg

SEQ ID NO: 26 - DNA, 1442 bp, Artificial Sequence
  Full 16sRNA of Ruminococcus bromii
ggcggcgtgcctaacacatgcaagtcgaacggaactgttttgaaagatttcttcggaatgaatttgatttagtttagtggcggacgggtgagtaacgcgtgagtaacctgccttcaagagggggataacattctgaaaagaatgctaataccgcatgacatatcggaaccacatggttctgatatcaaagattttatcgcttgaagatggactcgcgtccgattagttagttggtgaggtaacggctcaccaagaccgcgatcggtagccggactgagaggttgaacggccacattgggactgagacacggcccagactcctacgggaggcagcagtgggggatattgcgcaatgggggcaaccctgacgcagcaacgccgcgtgaaggatgaaggttttcggattgtaaacttcttttattaaggacgaaaaatgacggtacttaatgaataagctccggctaactacgtgccagcagccgcggtaatacgtagggagcaagcgttgtccggatttactgggtgtaaagggtgcgtaggcggctttgcaagtcagatgtgaaatctatgggctcaacccataaactgcatttgaaactgtagagcttgagtgaagtagaggcaggcggaattccccgtgtagcggtgaaatgcgtagagatggggaggaacaccagtggcgaaggcggcctgctgggctttaactgacgctgaggcacgaaagcgtgggtagcaaacaggattagataccctggtagtccacgctgtaaacgatgattactaggtgtggggggtctgaccccttccgtgccggagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatccaactaacgaagtagagatacattaggtgcccttcggggaaagttgagacaggtggtgcatggttgtcgtcagctcgtgtcgtgagatgttgggttaagtcccgcaacgagcgcaacccttgctattagttgctacgcaagagcactctaataggactgccgttgacaaaacggaggaaggtggggacgacgtcaaatcatcatgccccttatgacctgggctacacacgtactacaatggctgttaacagagggaagcaagacagtgatgtggagcaaacccctaaaaacattctcagttcagattgcaggctgcaacccgcctgcatgaagatggaattgctagtaatcgcggatcagaatgccgcggtgaatacgttcccgggccttgtacacaccgcccgtcacaccatgggagccggtaatacccgaagtcagtagtccaacctcgtgaggacgctgccgaaggtaggattggcgactggggtg

SEQ ID NO: 27 - DNA, 1451 bp, Artificial Sequence
  Full 16sRNA of Eubacterium hallii
gatgaacgctggcggcgtgcctaacacatgcaagtcgaacgaagcaccttaccwgattcttcggatgaaagwytggtgactgagtggcggacgggtgagtaacgcgtgggtaacctgccctgtacagggggataacagctggaaacggctgctaataccgcataagcgcacgaggagacatctccttgtgtgaaaaactccggtggtacaggatgggcccgcgtctgattagctggttggcagggtaacggcctaccaaggcaacgatcagtagccggtctgagaggatgaacggccacattggaactgagacacggtccaaactcctacgggaggcagcagtggggaatattgcacaatgggggaaaccctgatgcagcaacgccgcgtgagtgaagaagtatttcggtatgtaaagctctatcagcagggaagataatgacggtacctgactaagaagctccggctaaatacgtgccagcagccgcggtaatacgtatggagcaagcgttatccggatttactgggtgtaaagggtgcgtaggtggcagtgcaagtcagatgtgaaaggccggggctcaaccccggagctgcatttgaaactgctcggctagagtacaggagaggcaggcggaattcctagtgtagcggtgaaatgcgtagatattaggaggaacaccagtggcgaaggcggcctgctggactgttactgacactgaggcacgaaagcgtggggagcaaacaggattagataccctggtagtccacgccgtaaacgatgaatactaggtgtcggggccgtataggcttcggtgccgccgctaacgcagtaagtattccacctggggagtacgttcgcaagaatgaaactcaaaggaattgacggggacccgcacaagcggtggagcatgtggtttaattcgaagcaacgcgaagaaccttaccaggtcttgacatccttctgaccgcaccttaatcggtgctttccttcgggacagaagagacaggtggtgcatggttgtcgtcagctcgtgtcgtgagatgttgggttaagtcccgcaacgagcgcaacccctatcttcagtagccagcaggtaaggctgggcactctggagagactgccagggataacctggaggaaggtggggacgacgtcaaatcatcatgccccttatgatctgggcgacacacgtgctacaatggcggtcacagagtgaggcgaacccgcgagggggagcaaaccacaaaaaggccgtcccagttcggactgtagtctgcaacccgactacacgaagctggaatcgctagtaatcgcgaatcagaatgtcgcggtgaatacgttcccgggtcttgtacacaccgcccgtcacaccatgggagtcggaaatgcccgaagccagtgacccaacctttatggagggagctgtcgaaggtggagccggtaactgggg

SEQ ID NO: 28 - DNA, 1428 bp, Artificial Sequence
  Full 16sRNA of Roseburia inulinivorans
gatgaacgctggcggcgtgcttaacacatgcaagtcgaacgaagcacttttacagatttcttcggaatgaagttttagtgactgagtggcggacgggtgagtaacgcgtgggtaacctgcctcacacagggggataacagttggaaacggctgctaataccgcataagcgcacagtaccgcatggtacagtgtgaaaaactccggtggtgtgagatggacccgcgtctgattagctagttggcagggcaacggcctaccaaggcgacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaagaaatgacggtacctgactaagaagcaccggctaaatacgtgccagcagccgcggtaatacgtatggtgcaagcgttatccggatttactgggtgtaaagggagcgcaggcggaaggctaagtctgatgtgaaagcccggggctcaaccccggtactgcattggaaactggtcatctagagtgtcggaggggtaagtggaattcctagtgtagcggtgaaatgcgtagatattaggaggaacaccagtggcgaaggcggcttactggacgataactgacgctgaggctcgaaagcgtggggagcaaacaggattagataccctggtagtccacgccgtaaacgatgaatactaggtgtcggaaagcacagcttttcggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaagtcttgacatccttctgaccggacagtaatgtgtcctttccttcgggacagaagtgacaggtggtgcatggttgtcgtcagctcgtgtcgtgagatgttgggttaagtcccgcaacgagcgcaacccttatccccagtagccagcggttcggacgggcactctgaggagactgccagggataacctggaggaaggtggggatgacgtcaaatcatcatgccccttatgacttgggctacacacgtgctacaatggcgtaaacaaagggaagcgagaccgtgaggtggagcaaatcccaaaaataacgtctcagttcggactgtagtctgcaacccgactacacgaagctggaatcgctagtaatcgcagatcagaatgctgcggtgaatacgttcccgggtcttgtacacaccgcccgtcacaccatgggagttggaaatgcccgaagtcagtgacccaaccgcaaggagggagctg

SEQ ID NO: 29 - DNA, 1452 bp, Artificial Sequence
  Full 16sRNA of LT907848_s
gatgaacgctggcggcgtgcctaacacatgcaagtcgaacgaagcaccttttaagattcttcggatgattgatcggtgactgagtggcggacgggtgagtaacgcgtgggtaacctgccctgtacagggggataacagttggaaacggctgctaataccgcataagcgcacgagaggacatcctttcgtgtgaaaaactccggtggtacaggatgggcccgcgtctgattagctggttggcagggtaacggcctaccaaggcgacgatcagtagccggtctgagaggatgaacggccacattggaactgagacacggtccaaactcctacgggaggcagcagtggggaatattgcacaatgggggaaaccctgatgcagcaacgccgcgtgagtgaagaagtatttcggtatgtaaagctctatcagcagggaagataatgacggtacctgactaagaagctccggctaaatacgtgccagcagccgcggtaatacgtatggagcaagcgttatccggatttactgggtgtaaagggtgcgtaggtggcagtgcaagtcagatgtgaaaggccggggctcaaccccggagctgcatttgaaactgcatagctagagtacaggagaggcaggcggaattcctagtgtagcggtgaaatgcgtagatattaggaggaacaccagtggcgaaggcggcctgctggactgttactgacactgaggcacgaaagcgtggggagcaaacaggattagataccctggtagtccacgccgtaaacgatgaatactaggtgtcggggccgtataggcttcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaggtcttgacatccttctgaccactccgtaatgggagtcttccttcgggacagaagagacaggtggtgcatggttgtcgtcagctcgtgtcgtgagatgttgggttaagtcccgcaacgagcgcaacccctatcttcagtagccagcaggtaaggctgggcactctggagagactgccagggataacctggaggaaggtggggacgacgtcaaatcatcatgccccttatgatctgggcgacacacgtgctacaatggcggtcacaaagtgaggcaaacccgcgagggggagcaaaccacaaaaaggccgtcccagttcggactgtagtctgcaacccgactacacgaagctggaatcgctagtaatcgcgaatcagaatgtcgcggtgaatacgttcccgggtcttgtacacaccgcccgtcacaccatgggagtcggaaatgcccgaagccagtgacccaaccttttggagggagctgtcgaaggtggagccggtaactggggtg

SEQ ID NO: 30 - DNA, 1343 bp, Artificial Sequence
  Full 16sRNA of Roseburia cecicola group
  misc_feature (324)..(325): n is a, g, c or t
  misc_feature (360)..(361): n is a, g, c or t
  misc_feature (377): n is a, g, c or t
  misc_feature (576): n is a, g, c or t
  misc_feature (594): n is a, g, c or t
  misc_feature (633): n is a, g, c or t
  misc_feature (640): n is a, g, c or t
  misc_feature (846): n is a, g, c or t
  misc_feature (900): n is a, g, c or t
  misc_feature (902): n is a, g, c or t
  misc_feature (932): n is a, g, c or t
  misc_feature (1215): n is a, g, c or t
  misc_feature (1331): n is a, g, c or t
gatgaacgctggcggcgtgcttaacacatgcaagtcgaacgaagcactctatttgattttcttcggaaatgaagattttgtgactgagtggcggacgggtgagtaacgcgtgggtaacctgcctcatacagggggataacagttggaaacgactgctaataccgcataagcgcacagtactgcatggtaccgtgtgaaaaactccggtggtatgagatggacccgcgtctgattagccagttggcggggtaacggcccaccaaagcgacgatcagtagccgacctgagagggtgaccggccacattgggactgagacacggccnnaactcctrcgggaggcagcagtggggaatattgcnnaatgggggaaaccctnatgcagcgacgccgcgtgagcgaagaagtatttcggtatgtaaagctctatcagcagggaagaaaaatgacggtacctgactaagaagcaccggctaaatacgtgccagcagccgcggtaatacgtatggtgcmagcgttatycggatttactgggtgtmaagggagcgcmggcggtgcggcaagtctgatgtgaaagnccggggctymaccccggnactgcattggaaactgtcgtactagagtgtyggaggggnaagtggnattcctagtgtagcggtgaaatgcgtagatattaggaggaacaccagtggcgaaggcggcttactggacgattactgacgctgaggctcgaaagcgtggggagcaaacaggattagataccctggtagtccacgccgtaaacgatgaatactaggtgtcggggagcattgctcttcggtgccgcagcaaacgcwataagtattccncctggggagtacgttcgcaagaatgaaactcaaaggaattgacggggacccgcncnagcggtggagcatgtggtttaattcgaagnaacgcgaagaaccttaccaagtcttgacatccttctgacaatrtatgtaatgtatattctcttcggagcagaagtgacaggtggtgcatggttgtcgtcagctcgtgtcgtgagatgttgggttaagtcccgcaacgagcgcaaccctyattcttagtagccagcggttcggccgggcactctagggagactgccagggataacctggaggaaggtggggatgacgtcaaatcatcatgccccttatgacttgggctacacacgtgctacaatggcgtaaacnaagggaagcaagaccgtgaggtggagcaaaccccaaaaataacgtctcagttcggactgtagtctgcaactcgactacacgaagctggaatcgctagtaatcgcgaatcmgaatgncgcggtgaatac

SEQ ID NO: 31 - DNA, 1438 bp, Artificial Sequence
  Full 16sRNA of Clostridium celatum group
gacgaacgctggcggcgtgcctaacacatgcaagtcgagcgagtggatttccttcgggattgaagctagcggcggacgggtgagtaacacgtgggcaacctgcctcatagaggggaatagcctcccgaaagggagattaataccgcataagattgtagcttcgcatgaagtagcaattaaaggagcaatccgctatgagatgggcccgcggcgcattagctagttggtgaggtaacggctcaccaaggcgacgatgcgtagccgacctgagagggtgatcggccacattgggactgagacacggcccagactcctacgggaggcagcagtggggaatattgcacaatgggggaaaccctgatgcagcaacgccgcgtgagtgatgacggccttcgggttgtaaagctctgtcttcagggacgataatgacggtacctgaggaggaagccacggctaactacgtgccagcagccgcggtaatacgtaggtggcgagcgttgtccggatttactgggcgtaaagggagcgtaggcggacttttaagtgagatgtgaaatacccgggctcaacttgggtgctgcatttcaaactggaagtctagagtgcaggagaggagaatggaattcctagtgtagcggtgaaatgcgtagagattaggaagaacaccagtggcgaaggcgattctctggactgtaactgacgctgaggctcgaaagcgtggggagcaaacaggattagataccctggtagtccacgccgtaaacgatgaatactaggtgtaggggttgtcatgacctctgtgccgccgctaacgcattaagtattccgcctggggagtacggtcgcaagattaaaactcaaaggaattgacgggggcccgcacaagcagcggagcatgtggtttaattcgaagcaacgcgaagaaccttacctagacttgacatctcctgaattacccgtaactggggaagtcgcttcggcgacaggaagacaggtggtgcatggttgtcgtcagctcgtgtcgtgagatgttgggttaagtcccgcaacgagcgcaacccctattgttagttgctaccattaagttgagcactctagcgagactgcccgggttaaccgggaggaaggtggggatgacgtcaaatcatcatgccccttatgtctagggctacacacgtgctacaatggcaagtacaaagagaagcaagaccgcgaggtggagcaaaactcaaaaacttgtctcagttcggattgtaggctgaaactcgcctacatgaagctggagttgctagtaatcgcgaatcagcatgtcgcggtgaatacgttcccgggccttgtacacaccgcccgtcacaccatgagagttggcaatacccaaagtgcgtgatctgactcgcaagagaggaagcgccctaaggtagggtcagcgattggggtg

SEQ ID NO: 32 - DNA, 1441 bp, Artificial Sequence
  Full 16sRNA of PAC001046_s
gatgaacgctggcggcgtgcctaacacatgcaagtcgaacggaatttacatgaagcctagcgattgtaaatttagtggcggacgggtgagtaacgcgtgggtaacctgccttgtactgggggacaacagttggaaacgactgctaataccgcataagcgcacagcttcgcatgaagcagtgtgaaaaactccggtggtacaagatggacccgcgtctgattagctggttggtgaggtaacggcccaccaaggcgacgatcagtagccggcctgagagggtgaacggccacattgggactgagacacggcccaaactcctacgggaggcagcagtggggaatattgcacaatgggggaaaccctgatgcagcaacgccgcgtgagtgaagaagtatttcggtatgtaaagctctatcagcaggaaagaaaatgacggtacctgactaagaagccccggctaactacgtgccagcagccgcggtaatacgtagggggcaagcgttatccggatttactgggtgtaaagggagcgtagacggttttgcaagtctgaagtgaaagcccggggcttaaccccgggactgctttggaaactgtaggactagagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgattactaggtgttggtgggtatgacccatcggtgccgcagcaaacgcaataagtaatccacctggggagtacgttcgcaagaatgaaactcaaaggaattgacggggacccgcacaagcggtggagcatgtggtttaattcgaagcaacgcgaagaaccttacctggtcttgacatccctatgaataacgggcaatgccgttagtacttcggtacataggagacaggtggtgcatggttgtcgtcagctcgtgtcgtgagatgttgggttaagtcccgcaacgagcgcaacccttatctttagtagccagcagtaagatgggcactctagagagactgccggggataacccggaggaaggtggggatgacgtcaaatcatcatgccccttatgaccagggctacacacgtgctacaatggcgtaaacaaagagaagcgaagtcgtgaggcagagcgaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcggaaatgcccgaagtcggtgacctaaccgcaaggaaggagccgccgaaggcaggtctgataactggggtg

SEQ ID NO: 33 - DNA, 1448 bp, Artificial Sequence
  Full 16sRNA of Lactobacillus rogosae group
gatgaacgctggcggcgtgcttaacacatgcagtcgaacgaagcatttaagacagattacttcggtttgaagtcttttatgactgagtggcggacgggtgagtaacgcgtgggtaacctgcctcatacagggggatagcagctggaaacggctggtaataccgcataagcgcacagtaccacatggtacagtgtgaaaaactccggtggtatgagatggacccgcgtctgattagcttgttggcggggtaacggcccaccaaggcgacgatcagtagccgacctgagagggtgaccggccacattgggactgagacacggcccagactcctacgggaggcagcagtggggaatattgcacaatggaggaaactctgatgcagcgacgccgcgtgagtgaagaagtagttcgctatgtaaagctctatcagcagggaagatagtgacggtacctgactaagaagctccggctaaatacgtgccagcagccgcggtaatacgtatggagcaagcgttatccggatttactgggtgtaaagggagtgtaggtggccaggcaagtcagaagtgaaagcccggggctcaaccccgggactgcttttgaaactgcagggctagagtgcaggaggggcaagtggaattcctagtgtagcggtgaaatgcgtagatattaggaggaacaccagtggcgaaggcggcttgctggactgtaactgacactgaggctcgaaagcgtggggagcaaacaggattagataccctggtagtccacgccgtaaacgatgaatactaggtgtcggggcacataagtgctccggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcctcttgaccggtcagtaatgtgaccttttcttcggaacaagagtgacaggtggtgcatggttgtcgtcagctcgtgtcgtgagatgttgggttaagtcccgcaacgagcgcaacccttatccttagtagccagcagttcggctgggcactctagggagactgccagggataacctggaggaaggtggggatgacgtcaaatcatcatgccccttatgacttgggctacacacgtgctacaatggcgtaaacaaagtgaagcgagagtgtgagcttaagcaaatcacaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcggaaatgcccgaagtcggtgacctaacgtaagaagagccgccgaagcaggtctgataactggg

SEQ ID NO: 34 - DNA, 1449 bp, Artificial Sequence
  Full 16sRNA of Bacteroides uniformis
gatgaacgctagctacaggcttaacacatgcaagtcgaggggcagcatgaacttagcttgctaagtttgatggcgaccggcgcacgggtgagtaacacgtatccaacctgccgatgactcggggatagcctttcgaaagaaagattaatacccgatggcatagttcttccgcatggtggaactattaaagaatttcggtcatcgatggggatgcgttccattaggttgttggcggggtaacggcccaccaagccttcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagtgaggcacgtgtgcctttttgtatgtaccgtatgaataaggatcggctaactccgtgccagcagccgcggtaatacggaggatccgagcgttatccggatttattgggtttaaagggagcgtaggcggacgcttaagtcagttgtgaaagtttgcggctcaaccgtaaaattgcagttgatactgggtgtcttgagtacagtagaggcaggcggaattcgtggtgtagcggtgaaatgcttagatatcacgaagaactccgattgcgaaggcagcttgctggactgtaactgacgctgatgctcgaaagtgtgggtatcaaacaggattagataccctggtagtccacacagtaaacgatgaatactcgctgtttgcgatatacagtaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttgaattgcaactgaatgatgtggagacatgtcagccgcaaggcagttgtgaaggtgctgcatggttgtcgtcagctcgtgccgtgaggtgtcggcttaagtgccataacgagcgcaacccttatcgatagttaccatcaggttatgctggggactctgtcgagactgccgtcgtaagatgtgaggaaggtggggatgacgtcaaatcagcacggcccttacgtccggggctacacacgtgttacaatggggggtacagaaggcagctacacggcgacgtgatgctaatccctaaagcctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtgcgtaaccgcaaggagcgccctagggtaaaactggtgattggggct

SEQ ID NO: 35 - DNA, 1442 bp, Artificial Sequence; identical to SEQ ID NO: 26
  Full 16sRNA of Ruminococcus_g2

SEQ ID NO: 36 - DNA, 1453 bp, Artificial Sequence
  Full 16sRNA of Lachnospira
gatgaacgctggcggcgtgcttaacacatgcaagtcgaacgaagcaacwtatcacgattccttcgggatgacgatttgttgactgagtggcggacgggtgagtaacgcgtgggtaacctgccttatacagggggatagcagctggaaacggctgataataccgcataagcgcacggcatcgcatgatgcagtgtgaaaaactccggtggtataagatggacccgcgtctgattagctagttggtgaggtaacggcccaccaaggcaacgatcagtagccgacctgagagggtgaccggccacattgggactgagacacggcccagactcctacgggaggcagcagtggggaatattgcacaatggaggaaactctgatgcagcgacgccgcgtgagcgaagaagtatttcggtatgtaaagctctatcagcagggaagataatgacggtacctgactaagaagctccggctaaatacgtgccagcagccgcggtaatacgtatggagcaagcgttatccggatttactgggtgtaaagggagtgtaggtggcaaagcaagtcagtagtgaaaatccggggctcaacctcggaactgctattgaaactgtttagctagagtgcaggagaggtaagtggaattcctagtgtagcggtgaaatgcgtagatattaggaggaacaccagtggcgaaggcggcttactggactgtaactgacactgaggctcgaaagcgtggggagcaaacaggattagataccctggtagtccacgccgtaaacgatgaatactaggtgttgggtctcataagagattcggtgccgcagctaacgcaataagtattccacctggggagtacgttcgcaagaatgaaactcaaaggaattgacggggacccgcacaagcggtggagcatgtggtttaattcgaagcaacgcgaagaaccttacctagtcttgacatcccgatgaccragtatgtaatgtactctttcttcggaacatcggtgacaggtggtgcatggttgtcgtcagctcgtgtcgtgagatgttgggttaagtcccgcaacgagcgcaacccctatttctagtagccagcagttcggctgggcactctagagagactgccagggataacctggaggaaggtggggatgacgtcaaatcatcatgccccttatgactagggctacacacgtgctacaatggcgtaaacaaagtgaagcgagagtgtgagcttaagcaaatcacaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcgaaaatgcccgaagtcggtgacctaacgtaagaaggagccgccgaaggcaggtttgataactggggtg

SEQ ID NO: 37 - DNA, 1451 bp, Artificial Sequence
  Full 16sRNA of Bacteroides
gatgaacgctagctacaggcttaacacatgcaagtcgaggggcatcaggaagaaagcttgctttctttgctggcgaccggcgcacgggtgagtaacacgtatccaacctgccctttactcggggatagcctttcgaaagaaagattaatacccgatagcataatgattccgcatggtttcattattaaaggattccggtaaaggatggggatgcgttccattaggttgttggtgaggtaacggctcaccaagccttcgatggataggggttctgagaggaaggtcccccacattggaactgagacacggtccaaactcctacgggaggcagcagtgaggaatattggtcaatgggcgctagcctgaaccagccaagtagcgtgaaggatgaaggctctatgggtcgtaaacttcttttatataagaataaagtgcagtatgtatactgttttgtatgtattatatgaataaggatcggctaactccgtgccagcagccgcggtaatacggaggatccgagcgttatccggatttattgggtttaaagggagcgtaggtggactggtaagtcagttgtgaaagtttgcggctcaaccgtaaaattgcagttgatactgtcagtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcagtggaatgatgtggaaacatgtcagtgagcaatcaccgctgtgaaggtgctgcatggttgtcgtcagctcgtgccgtgaggtgtcggcttaagtgccataacgagcgcaacccttatctttagttactaacaggttatgctgaggactctagagagactgccgtcgtaagatgtgaggaaggtggggatgacgtcaaatcagcacggcccttacgtccggggctacacacgtgttacaatggggggtacagaaggcagctagcgggtgaccgtatgctaatcccaaaatcctctctcagttcggatcgaagtctgcaacccgacttcgtgaagctggattcgctagtaatcgcgcatcagccacggcgcggtgaatacgttcccgggccttgtacacaccgcccgtcaagccatgggagccgggggtacctgaagtacgtaaccgcaaggatcgtcctagggtaaaactggtgactggggct

SEQ ID NO: 38 - DNA, 1431 bp, Artificial Sequence; identical to SEQ ID NO: 20
  Full 16sRNA of Faecalibacterium

SEQ ID NO: 39 - DNA, 1451 bp, Artificial Sequence; identical to SEQ ID NO: 27
  Full 16sRNA of Eubacterium_g5

SEQ ID NO: 40 - DNA, 1415 bp, Artificial Sequence; identical to SEQ ID NO: 19
  Full 16sRNA of Fusicatenibacter

SEQ ID NO: 41 - DNA, 1343 bp, Artificial Sequence; identical to SEQ ID NO: 30
  Full 16sRNA of Roseburia
  misc_feature (324)..(325): n is a, g, c or t
  misc_feature (360)..(361): n is a, g, c or t
  misc_feature (377): n is a, g, c or t
  misc_feature (576): n is a, g, c or t
  misc_feature (594): n is a, g, c or t
  misc_feature (633): n is a, g, c or t
  misc_feature (640): n is a, g, c or t
  misc_feature (846): n is a, g, c or t
  misc_feature (900): n is a, g, c or t
  misc_feature (902): n is a, g, c or t
  misc_feature (932): n is a, g, c or t
  misc_feature (1215): n is a, g, c or t
  misc_feature (1331): n is a, g, c or t

SEQ ID NO: 42 - DNA, 1451 bp, Artificial Sequence
  Full 16sRNA of Subdoligranulum
gacgaacgctggcggcgcgcctaacacatgcaagtcgaacggagttatttcggttgaagttttcggatggatactggtttaacttagtggcgaacgggtgagtaacgcgtgagtaacctgccctggagtgggggacaacagttggaaacgactgctaataccgcataagcccacggcccggcatcgggctgagggaaaaggatttattcgcttcaggatggactcgcgtccaattagctagttggtgaggtaacggcccaccaaggcgacgattggtagccggactgagaggttgaacggccacattgggactgagacacggcccagactcctacgggaggcagcagtgggggatattgcacaatgggggaaaccctgatgcagcgacgccgcgtggaggaagaaggttttcggattgtaaactcctgtcgttagggacgaatcttgacggtacctaacaagaaagcaccggctaactacgtgccagcagccgcggtaaaacgtagggtgcaagcgttgtccggaattactgggtgtaaagggagcgcaggcggaccggcaagttggaagtgaaatctatgggctcaacccataaattgctttcaaaactgctggccttgagtagtgcagaggtaggtggaattcccggtgtagcggtggaatgcgtagatatcgggaggaacaccagtggcgaaggcgacctactgggcaccaactgacgctgaggctcgaaagcatgggtagcaaacaggattagataccctggtagtccatgccgtaaacgatgattactaggtgttggaggattgaccccttcagtgccgcagttaacacaataagtaatccacctggggagtacgaccgcaaggttgaaactcaaaggaattgacgggggcccgcacaagcagtggagtatgtggtttaattcgaagcaacgcgaagaaccttaccaggtcttgacatccgatgcatagtgcagagatgcatgaagtccttcgggacatcgagacaggtggtgcatggttgtcgtcagctcgtgtcgtgagatgttgggttaagtcccgcaacgagcgcaacccttattgccagttactacgcaagaggactctggcgagactgccgttgacaaaacggaggaaggtggggatgacgtcaaatcatcatgccctttatgacctgggctacacacgtactacaatggcgtttaacaaagagaagcaagaccgcgaggtggagcaaaactcaaaaacaacgtctcagttcagattgcaggctgcaactcgcctgcatgaagtcggaattgctagtaatcgcggatcagcatgccgcggtgaatacgttcccgggccttgtacacaccgcccgtcacaccatgagagccggggggacccgaagtcggtagtctaaccgcaaggaggacgccgccgaaggtaaaactggtgattggggtg

SEQ ID NO: 43 - DNA, 1452 bp, Artificial Sequence
  Full 16sRNA of Blautia
  misc_feature (4): n is a, g, c or t
  misc_feature (164): n is a, g, c or t
  misc_feature (166): n is a, g, c or t
  misc_feature (181)..(182): n is a, g, c or t
  misc_feature (237): n is a, g, c or t
  misc_feature (322)..(323): n is a, g, c or t
  misc_feature (457): n is a, g, c or t
  misc_feature (506)..(508): n is a, g, c or t
  misc_feature (557): n is a, g, c or t
  misc_feature (579): n is a, g, c or t
  misc_feature (649): n is a, g, c or t
  misc_feature (727): n is a, g, c or t
  misc_feature (754): n is a, g, c or t
  misc_feature (776): n is a, g, c or t
  misc_feature (780): n is a, g, c or t
  misc_feature (890): n is a, g, c or t
  misc_feature (892): n is a, g, c or t
  misc_feature (928)..(929): n is a, g, c or t
  misc_feature (1072): n is a, g, c or t
  misc_feature (1132): n is a, g, c or t
  misc_feature (1155): n is a, g, c or t
  misc_feature (1158)..(1159): n is a, g, c or t
  misc_feature (1213): n is a, g, c or t
  misc_feature (1254): n is a, g, c or t
  misc_feature (1281): n is a, g, c or t
  misc_feature (1323): n is a, g, c or t
  misc_feature (1340): n is a, g, c or t
  misc_feature (1351): n is a, g, c or t
gatnaacgctggcggcgtgcttaacacatgcaagtcgagcgaagcgctaagacagatttcttcggattgaagtctttgtgacttagcggcggacgggtgagtaacgcgtgggtaacctgcctcatacagggggataacagttagaaatgactgctaataccgcntnagcgcacaggaccgnntggtctggtgtgaaaaactccggtggtatgagatggacccgcgtctgattagctngttggaggggtaacggcccaccaaggcgacgatcagtagccggcctgagagggtgaacggccacattgggactgagacacggccnngactcctacgggaggcagcagtggggaatattgcacaatgggggaaaccctgatgcagcgacgccgcgtgaaggaagaagtatctcggtatgtaaacttctatcagcagggaagaaaatgacggtacctgactnagaagccccggctaactacgtgccagcagccgcggtaatacgtaggggnnnagcgttatccggatttactgggtgtaaagggagcgtagacggaagagcnagtctgatgtgaaaggctgggncttaaccccaggactgcattggaaactgttgttcgagagtgccggagaggtaagcggaattcctagtgtngcggtgaaatgcgtagatattaggaggaacaccagtggcgaaggcggcttactggacggtaactgacgttgaggctcnaaagcgtggggagcaaacaggattagntaccctggtagtccacgccgtnaacnatgaatactaggtgtcgggtggcaaagccattcggtgccgcagcaaacgcaataagtattccacctggggagtacgttcgcaagaatgaaactcaaaggaattgacgggnanccgcacaagcggtggagcatgtggtttaattcgaannaacgcgaagaaccttaccaagtcttgacatccctctgaccgtcccgtaacgggggcttcccttcggggcagaggagacaggtggtgcatggttgtcgtcagctcgtgtcgtgagatgttgggttaagtcccgcaacgagcgcnacccttatccttagtagccagcacatgatggtgggcactctagggagactgccggggatnacccggaggaaggcggggacgangtnnaatcatcatgccccttatgatttgggctacacacgtgctacaatggcgtaaacnaagggaagcgagacagcgatgttgagcgaatcccaaaaatnacgtcccagttcggactgcagtctgcnactcgactgcacgaagctggaatcgctagtaatcgcggatcngaatgccgcggtgaatncgttcccgggncttgtacacaccgcccgtcacaccatgggagtcagtaacgcccgaagtcagtgacctaaccgaaaggaaggagctgccgaaggcgggaccgataactgggg

SEQ ID NO: 44 - DNA, 1447 bp, Artificial Sequence
  Full 16sRNA of CCMM_g
gatgaacgctggcggcgtgcctaatacatgcaagtcgaacgcttcacttcggtgaagagtggcgaacgggtgagtaatacataagtaacctggcatctacagggggataactgatggaaacgtcagctaagaccgcataggtgtagagatcgcatgaactctatatgaaaagtgctacgggactggtagatgatggacttatggcgcattagcttgttggtagggtaacggcctaccaaggcgacgatgcgtagccgacctgagagggtgaccggccacactgggactgagacacggcccagactcctacgggaggcagcagtagggaattttcggcaatgggggaaaccctgaccgagcaacgccgcgtgaaggaagaagtaattcgttatgtaaacttctgtcatagaggaagaacggtggatatagggaatgatatccaagtgacggtactctataagaaagccacggctaactacgtgccagcagccgcggtaatacgtaggtggcgagcgttatccggaattattgggcgtaaagagggagcaggcggcactaagggtctgtggtgaaagatcgaagcttaacttcggtaagccatggaaaccgtagagctagagtgtgtgagaggatcgtggaattccatgtgtagcggtgaaatgcgtagatatatggaggaacaccagtggcgaaggcgacgatctggcgcataactgacgctcagtcccgaaagcgtggggagcaaataggattagataccctagtagtccacgccgtaaacgatgagtactaagtgttgggtgtcaaagctcagtgctgcagttaacgcaataagtactccgcctgagtagtacgttcgcaagaatgaaactcaaaggaattgacgggggcccgcacaagcggtggagcatgtggtttaattcgaagcaacgcgaagaaccttaccaggtcttgacatcgatctaaaggctccagagatggagagatagctatagagaagacaggtggtgcatggttgtcgtcagctcgtgtcgtgagatgttgggttaagtcccgcaacgagcgcaacccctgttgccagttgccagcattaagttggggactctggcgagactgccggtgacaagccggaggaaggcggggatgacgtcaaatcatcatgccccttatgacctgggctacacacgtgctacaatggacagagcagagggaagcgaagccgcgaggtggagcgaaacccataaaactgttctcagttcggactgcagtctgcaactcgactgcacgaagatggaatcgctagtaatcgcgaatcagcatgtcgcggtgaatacgttctcgggccttgtacacaccgcccgtcacaccatgagagtcggtaacacccgaagccggtggcctaaccgcaaggaaggagctgtctaaggtgggactgatgattggggtg

SEQ ID NO: 45 - DNA, 1455 bp, Artificial Sequence
  Full 16sRNA of Agathobacter
gatgaacgctggcggcgtgcttaacacatgcaagtcgaacgaagcactttatttgatttccttcgggactgattattttgtgactgagtggcggacgggtgagtaacgcgtgggtaacctgccttgtacagggggataacagttggaaacggctgctaataccgcataagcgcacggcatcgcatgatgcagtgtgaaaaactccggtggtataagatggacccgcgttggattagctagttggtgaggtaacggcccaccaaggcgacgatccatagccgacctgagagggtgaccggccacattgggactgagacacggcccaaactcctacgggaggcagcagtggggaatattgcacaatgggcgaaagcctgatgcagcgacgccgcgtgagcgaagaagtatttcggtatgtaaagctctatcagcagggaagataatgacggtacctgactaagaagcaccggctaaatacgtgccagcagccgcggtaatacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacaggattagataccctggtagtccacgccgtaaacgatgaatactaggtgttgggaagcattgcttctcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccttctgaccggtacttaaccgtaccttctcttcggagcaggagtgacaggtggtgcatggttgtcgtcagctcgtgtcgtgagatgttgggttaagtcccgcaacgagcgcaacccttatctttagtagccagcggttcggccgggcactctagagagactgccagggataacctggaggaaggcggggatgacgtcaaatcatcatgccccttatgacttgggctacacacgtgctacaatggcgtaaacaaagggaagcaaagctgtgaagccgagcaaatctcaaaaataacgtctcagttcggactgtagtctgcaacccgactacacgaagctggaatcgctagtaatcgcagatcagaatgctgcggtgaatacgttcccgggtcttgtacacaccgcccgtcacaccatgggagttgggaatgcccgaagccagtgacctaaccgaaaggaaggagctgtcgaaggcaggctcgataactggggtg

SEQ ID NO: 46 - DNA, 1464 bp, Artificial Sequence
  Full 16sRNA of Parasutterella
attgaacgctggcggaacgctttacacatgcaagtcgaacggtaacgcggagagaagcttgcttctctccggcgacgagtggcgaacgggtgagtaatacatcggaacgtgtccgctcgtgggggacaaccagccgaaaggttggctaataccgcatgagttctacggaagaaagagggggacccgcaagggcctctcgcgagcggagcggccgatgactgattagcctgttggtgaggtaacggctcaccaaagcaacgatcagtagctggtctgagaggacgaccagccacactgggactgagacacggcccagactcctacgggaggcagcagtggggaattttggacaatgggcgcaagcctgatccagctattccgcgtgtgggatgaaggccctcgggttgtaaaccacttttgtagagaacgaaaagacaccttcgaataaagggtgttgctgacggtactctaagaataagcaccggctaactacgtgccagcagccgcggtaatacgtagggtgcgagcgttaatcggaattactgggcgtaaagggtgcgcaggcggttgagtaagacagatgtgaaatccccgagcttaactcgggaatggcatatgtgactgctcgactagagtgtgtcagagggaggtggaattccacgtgtagcagtgaaatgcgtagatatgtggaagaacaccgatggcgaaggcagcctcctgggacataactgacgctcaggcacgaaagcgtggggagcaaacaggattagataccctggtagtccacgccctaaacgatgttaactagttgttgggaagtaaaattctcagtaacgcagccaacgcgagaagttaaccgcctgggaagtacggtcgcaagactaaaactcaaaggaattgacggggacccgcacaagcggtggatgatgtggattaattcgatgcaacgcgaaaaaccttacctacccttgacatgtcaggaagctcttgtaatgagagcgtgcccgcaagggagcctgaacacaggtgctgcatggctgtcgtcagctcgtgtcgtgagatgttgggttaagtcccgcaacgagcgcaacccttgtcactagttgctacgaaagggcactctagtgagactgccggtgacaaaccggaggaaggtggggatgacgtcaagtcctcatggcccttatgggtagggcttcacacgtcatacaatggtcggaacagagggcagcgaagccgtgaggcggagccaatcccagaaaaccgatcgtagtccggattgcagtctgcaactcgactgcatgaagtcggaatcgctagtaatcgcggatcagcatgccgcggtgaatacgttcccgggtcttgtacacaccgcccgtcaaacaatgggagtggtgtttaccagaagtcgttagcctaaccgcaaggagggcggcgaccacggtgagcaccgtgactaatgtt

SEQ ID NO: 47 - DNA, 1429 bp, Artificial Sequence
  Full 16sRNA of Romboutsia
gatgaacgctggcggcgtgcctaacacatgcaagtcgagcgatttacttcggtaaagagcggcggacgggtgagtaacgcgtgggtaacctgccctgtacacacggataacgtaccgaaaggtatgctaatacgagataaaatacttttgtcgcatggtagaagtatcaaagcttttgcggtacaggatggacccgcgtctgattagctagttggtaaggtaacggcttaccaaggcgacgatcagtagccgacctgagagggtgatcggccacattggaactgagacacggtccaaactcctacgggaggcagcagtggggaatattgcacaatgggcgaaagcctgatgcagcaacgccgcgtgagcgatgaaggccttcgggtcgtaaagctctgtcctcaaggaagataatgacggtacttgaggaggaagccccggctaactacgtgccagcagccgcggtaatacgtagggggctagcgttattccgaaattactgggcgaaaagggtgcgtagggtggtttctaaagtcagaggtgaaaggctacggctcaaccgtagtaagcctttgaaactggggaacttgagtgcaggagaggagagtggaattcctagtgtagcggtgaaatgcgtagatattaggaggaacaccagttgcgaaggcggctctctggactgtaactgacactgaggcacgaaagcgtggggagcaaacaggattagataccctggtagtccacgccgtaaacgatgagtactagctgtcggaggttacccccttcggtggcgcagctaacgcattaagtactccgcctgggaagtacgctcgcaagagtgaaactcaaaggaattgacggggacccgcacaagtagcggagcatgtggtttaattcgaagcaacgcgaagaaccttacctaagcttgacatccttttgaccgatgcctaatcgcatctttcccttcggggacagaagtgacaggtggtgcatggttgtcgtcagctcgtgtcgtgagatgttgggttaagtcccgcaacgagcgcaacccttgcctttagttgccagcattaagttgggcactctagagggactgccagggataacctggaggaaggtggggatgacgtcaaatcatcatgccccttatgcttagggctacacacgtgctacaatgggtggtacagagggcagccaagtcgtgaggcggagctaatcccttaaagccattctcagttcggattgtaggctgaaactcgcctacatgaagctggagttactagtaatcgcagatcagaatgctgcggtgaatgcgttcccgggtcttgtacacaccgcccgtcacaccacggaagttgggggcgcccgaagccacttagctaacccttttgggaagcgagtgtcgaaggtgaaatcaataactggggtg

SEQ ID NO: 48 - DNA, 1441 bp, Artificial Sequence; identical to SEQ ID NO: 32
  Full 16sRNA of PAC001046_g

SEQ ID NO: 49 - DNA, 1439 bp, Artificial Sequence
  Full 16sRNA of Eubacterium_g23
gtgtgcctaacacatacaagtcagtcgacgagcttgacgaacgattcttcggatgaattctgatatgactgagtggcggacgggtgagtaacgcgtgagcaacctgcccttcagagggggatagcgtctggaaacggacggtaataccgcataatgtacaatgatggcatcattgatgtaccaaagctattgcgctgaaggatgggctcgcgtctgattagatagttggtggggtaacggcctaccaagtcgacgatcagtagccggactgagaggttgaacggccacattgggactgagacacggcccagactcctacgggaggcagcagtggggaatattgcacaatgggcgcaagcctgatgcagcaacgccgcgtggaggaagacggttttcggattgtaaactcctgttcttagtgaagaaaaatgacggtagctaaggagcaagccacggctaactacgtgccagcagccgcggtaatacgtaggtggcaagcgttgtccggaattactgggtgtaaagggagcgcaggcgggggagcaagtcagctgtgaaatctatgggcttaacccataaactgcagttgaaactgttcttcttgagtgaagtagaggttggcggaattccgagtgtagcggtgaaatgcgtagatattcggaggaacaccggtggcgaaggcggccaactgggcttttactgacgctgaggctcgaaagtgtggggagcaaacaggattagataccctggtagtccacactgtaaacgatgataactaggtgtagggggtctgaccccttctgtgccgcagctaacgcaataagttatccacctggggagtacgaccgcaaggttgaaactcaaaggaattgacggggacccgcacaagcagtggattatgtggtttaattcgatgcaacgcgaagaaccttaccagcacttgacatccaactaacgaaatagagatatattaggtgcccctcggggaaagttgagacaggtggtgcatggttgtcgtcagctcgtgtcgtgagatgttgggttaagtcccgcaacgagcgcaacccctgccattagttgctacgcaagagcactctaatgggaccgctaccgacaaggtggaggaaggtggggatgacgtcaaatcatcatgccccttatgtgctgggctacacacgtaatacaatggtcgttaacaaagagaagcaataccgcgaggtggagcaaaacttcaaaaacgatctcagttcggactgtaggctgaaactcgcctgcacgaagttggaattgctagtaatcgtggatcagcatgccacggtgaatacgttcccgggtcttgtacacaccgcccgtcacaccatgggagccggtaatacccgaagtcagtagtctaaccttaatggaggacgctgccgaaggtaggattggcgactggggtg

SEQ ID NO: 50 - DNA, 1462 bp, Artificial Sequence
  Full 16sRNA of FWNZ_s
attgaacgctggcggcaggcctaacacatgcaagtcgagcggtagcacagagagcttgctctcgggtgacgagcggcggacgggtgagtaatgtctgggaaactgcctgatggagggggataactactggaaacggtagctaataccgcataacgtcgcaagaccaaagtgggggaccttcgggcctcatgccatcagatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcggggaggaaggcggtgaggttaataacctcatcgattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgatttggaggttgtgcccttgaggcgtggcttccggagctaacgcgttaaatcgaccgcctggggagtacggccgcaaggttaaaactcaaatgaattgacgggggcccgcacaagcggtggagcatgtggtttaattcgatgcaacgcgaagaaccttacctggtcttgacatccacagaacttagcagagatgctttggtgccttcgggaactgtgagacaggtgctgcatggctgtcgtcagctcgtgttgtgaaatgttgggttaagtcccgcaacgagcgcaacccttatcctttgttgccagcggttaggccgggaactcaaaggagactgccagtgataaactggaggaaggtggggatgacgtcaagtcatcatggcccttacgaccagggctacacacgtgctacaatggcatatacaaagagaagcgacctcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtg

SEQ ID NO: 51 - DNA, 1454 bp, Artificial Sequence
  Full 16sRNA of Flavonifractor plautii
gatgaacgctggcggcgtgcttaacacatgcaagtcgaacggggtgctcatgacggaggattcgtccaatggattgagttacctagtggcggacgggtgagtaacgcgtgaggaacctgccttggagaggggaataacactccgaaaggagtgctaataccgcatgaagcagttgggtcgcatggctctgactgccaaagatttatcgctctgagatggcctcgcgtctgattagctagtaggcggggtaacggcccacctaggcgacgatcagtagccggactgagaggttgaccggccacattgggactgagacacggcccagactcctacgggaggcagcagtggggaatattgggcaatgggcgcaagcctgacccagcaacgccgcgtgaaggaagaaggctttcgggttgtaaacttcttttgtcggggacgaaacaaatgacggtacccgacgaataagccacggctaactacgtgccagcagccgcggtaatacgtaggtggcaagcgttatccggatttactgggtgtaaagggcgtgtaggcgggattgcaagtcagatgtgaaaactgggggctcaacctccagcctgcatttgaaactgtagttcttgagtgctggagaggcaatcggaattccgtgtgtagcggtgaaatgcgtagatatacggaggaacaccagtggcgaaggcggattgctggacagtaactgacgctgaggcgcgaaagcgtggggagcaaacaggattagataccctggtagtccacgccgtaaacgatggatactaggtgtggggggtctgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcccactaacgaggcagagatgcgttaggtgcccttcggggaaagtggagacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttatgtcctgggccacacacgtactacaatggtggttaacagagggaggcaataccgcgaggtggagcaaatccctaaaagccatcccagttcggattgcaggctgaaacccgcctgtatgaagttggaatcgctagtaatcgcggatcagcatgccgcggtgaatacgttcccgggccttgtacacaccgcccgtcacaccatgagagtcgggaacacccgaagtccgtagcctaaccgcaaggagggcgcggccgaaggtgggttcgataattggggtg

SEQ ID NO: 52 - DNA, 1471 bp, Artificial Sequence
  Full 16sRNA of Streptococcus gallolyticus group
gacgaacgctggcggcgtgcctaatacatgcaagtagaacgctgactactttagcttgctagagtagaaggagttgcgaacgggtgagtaacgcgtaggtaacctgcctactagcgggggataactattggaaacgatagctaataccgcataacagtgtttaacacatgttagatgcttgaaagatgcaaatgcatcactagtagatggacctgcgttgtattagctagttggtggggtaacggcctaccaaggcgacgatacatagccgacctgagagggtgatcggccacactgggactgagacacggcccagactcctacgggaggcagcagtagggaatcttcggcaatgggggcaaccctgaccgagcaacgccgcgtgagtgaagaaggttttcggatcgtaaagctctgttgtaagagaagaacgtgtgtgagagtggaaagttcacacagtgacggtaacttaccagaaagggacggctaactacgtgccagcagccgcggtaatacgtaggtcccgagcgttgtccggatttattgggcgtaaagcgagcgcaggcggtttaataagtctgaagttaaaggcagtggcttaaccattgttcgctttggaaactgttaaacttgagtgcagaaggggagagtggaattccatgtgtagcggtgaaatgcgtagatatatggaggaacaccggtggcgaaagcggctctctggtctgtaactgacgctgaggctcgaaagcgtggggagcaaacaggattagataccctggtagtccacgccgtaaacgctgagtgctaggtgttaggccctttccggggcttagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcccgatgctatttctagagatagaaagtttcttcggaacatcggtgacaggtggtgcatggttgtcgtcagctcgtgtcgtgagatgttgggttaagtcccgcaacgagcgcaacccctattgttagttgccatcattgagttgggcactctagcgagactgccggtaataaaccggaggaaggtggggatgacgtcaaatcatcatgccccttatgacctgggctacacacgtgctacaatggttggtacaacgagtcgcaagtcggtgacggcaagcaaatctcttaaagccaatctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccttttaggagccagccgcctaaggtgggatagatgattggggtg

SEQ ID NO: 53 - DNA, 1437 bp, Artificial Sequence
  Full 16sRNA of Clostridium neonatale
gacgaacgctggcggcgtgcctaacacatgcaagtcgagcgatgaagtttccttcgggaaacggattagcggcggacgggtgagtaacacgtgggtaacctgccttatagtgggggatagcctttcgaaaggaagattaataccgcataagattgtagtatcgcatgatatagcaattaaaggagtaatccgctataagatggacccgcgtcgcattagctagttggtgaggtaatggctcaccaaggcgacgatgcgtagccgacctgagagggtgatcggccacattgggactgagacacggcccagactcctacgggaggcagcagtggggaatattgcacaatgggcgaaagcctgatgcagcaacgccgcgtgagtgatgacggccttcgggttgtaaaactctgtcttcagggacgataatgacggtacctgaggaggaagccacggctaactacgtgccagcagccgcggtaatacgtaggtggcaagcgttgtccggatttactgggcgtaaagggagcgtaggcggatgtttaagtgggatgtgaaatactcgggctcaacttgagtgctgcattccaaactggatatctagagtgcaggagaggaaaggagaattcctagtgtagcggtgaaatgcgtagagattaggaagaataccagtggcgaaggcgcctttctggactgtaactgacgctgaggctcgaaagcgtggggagcaaacaggattagataccctggtagtccacgccgtaaacgatgaatactaggtgtaggggttgtcatgacctctgtgccgccgctaacgcattaagtattccgcctggggagtacggtcgcaagattaaaactcaaaggaattgacgggggcccgcacaagcagcggagcatgtggtttaattcgaagcaacgcgaagaaccttacctagacttgacatctcctgaattactctgtaatggaggaagctcttcggagcaggaagacaggtggtgcatggttgtcgtcagctcgtgtcgtgagatgttgggttaagtcccgcaacgagcgcaacccttattgttagttgctaccatttagttgagcactctagcgagactgcccgggttaaccgggaggaaggtggggatgacgtcaaatcatcatgccccttatgtctagggctacacacgtgctacaatggccggtacagtaagatgcaataccgtgaggtggagcaaaactcaaaaaccggtctcagttcggattgtaggctgaaactcgcctacatgaagctggagttgctagtaatcgcgaatcagaatgtcgcggtgaatacgttcccgggccttgtacacaccgcccgtcacaccatgagagttggcaatacccaaagttcgtgagctaacgcgtaagcgaggcagcgacctaaggtagggtcagcgattggggtg

SEQ ID NO: 54 - DNA, 1425 bp, Artificial Sequence
  Full 16sRNA of Clostridioides difficile group
gatgaacgctggcggcgtgcctaacacatgcaagttgagcgatttacttcggtaaagagcggcggacgggtgagtaacgcgtgggtaacctaccctgtacacacggataacataccgaaaggtatgctaatacgggataatatatttgagaggcatctcttgaatatcaaaggtgagccggtacaggatggacccgcgtctgattagctagttggtaaggtaacggcttaccaaggcgacgatcagtagccgacctgagagggtgatcggccacattggaactgagacacggtccaaactcctacgggaggcagcagtggggaatattgcacaatgggcgaaagcctgatgcagcaacgccgcgtgagtgatgaaggccttcgggtcgtaaaactctgtcctcaaggaagataatgacggtacttgaggaggaagccccggctaactacgtgccagcagccgcggtaatacgtagggggctagcgttatccggatttactgggcgtaaagggtgcgtaggcggtctttcaagtcaggagtgaaaggctacggctcaaccgtagtaagctcttgaaactgggagacttgagtgcaggagaggagagtggaattcctagtgtagcggtgaaatgcgtagatattaggaggaacaccagttgcgaaggcggctctctggactgtaactgacgctgaggcacgaaagcgtggggagcaaacaggattagataccctggtagtccacgctgtaaacgatgagtactaggtgtcgggggttacccccctcggtgccgcagctaacgcattaagtactccgcctgggaagtacgctcgcaagagtgaaactcaaaggaattgacggggacccgcacaagtagcggagcatgtggtttaattcgaagcaacgcgaagaaccttacctaagcttgacatcccaatgacatctccttaatcggagagttcccttcggggacattggtgacaggtggtgcatggttgtcgtcagctcgtgtcgtgagatgttgggttaagtcccgcaacgagcgcaacccttgtctttagttgccatcattaagttgggcactctagagagactgccagggataacctggaggaaggtggggatgacgtcaaatcatcatgccccttatgcttagggctacacacgtgctacaatgggtagtacagagggttgccaagccgtaaggtggagctaatcccttaaagctactctcagttcggattgtaggctgaaactcgcctacatgaagctggagttactagtaatcgcagatcagaatgctgcggtgaatgcgttcccgggtcttgtacacaccgcccgtcacaccacgggagttggagacgcccgaagccgattatctaaccttttggaagaagtcgtcgaaggtggaatcaataactggggtg

SEQ ID NO: 55 - DNA, 1396 bp, Artificial Sequence
  Full 16sRNA of Veillonella ratti group
aaagtggaagcttgcttctagcgatcttagtggcgaacgggtgagtaacgcgtaaccaacctgcccttcagagggggataacaacgggaaaccgttgctaataccgcgtacgaatgaacttcggcatcggagctcattgaaaggtggcctctatttataagctatcgctgaaggagggggttgcgtctgattagctagttggaggggtaacggcccaccaaggcaatgatcagtagccggtctgagaggatgaacggccacattgggactgagacacggcccaaactcctacgggaagcagcagtggggaatcttccgcaatggacgaaagtctgacggagcaacgccgcgtgagtgatgacggccttcgggttgtaaagctctgttaatcgggacgaatggtctttgtgtgaataatgcaaagatttgacggtaccggaatagaaagccacggctaactacgtgccagcagccgcggtaatacgtaggtggcaagcgttgtccggaattattgggcgtaaagcgcgcgcaggcggtttcataagtctgtcttaaaagtgcggggcttaaccccgtgaggggatggaaactatggaactggagtatcggagaggaaagcggaattcctagtgtagcggtgaaatgcgtagatattaggaagaacaccagtggcgaaggcggctttctggacgacaactgacgctgaggcgcgaaagccaggggagcgaacgggattagataccccggtagtcctggccgtaaacgatgggtactaggtgtaggaggtatcgaccccttctgtgccggagttaacgcaataagtaccccgcctggggagtacggtcgcaaggctgaaactcaaaggaattgacgggggcccgcacaagcggtggagtatgtggtttaattcgacgcaacgcgaagaaccttaccaggtcttgacattgatggacgaaacaagagattgtttttctccttcgggagccagaaaacaggtggtgcacggctgtcgtcagctcgtgtcgtgagatgttgggttaagtcccgcaacgagcgcaacccctatcttatgttgccagcacttcgggtgggaactcatgagagactgccgcagacaatgcggaggaaggcggggatgacgtcaagtcatcatgccccttatgacctgggctacacacgtactacaatgggctttaatagagggaagcgaaaccgcgaggtggagcaaaccccagaaacaagctctcagttcggatcgtaggctgcaactcgcctacgtgaagtcggaatcgctagtaatcgcaggtcagcatactgcggtgaatacgttcccgggccttgtacacaccgcccgtcacaccacgaaagtcggaagtacccaaagccggtggggtaa

SEQ ID NO: 56 - DNA, 1464 bp, Artificial Sequence
  Full 16sRNA of Escherichia coli group
attgaacgctggcggcaggcctaacacatgcaagtcgaacggtaacagaaagcagcttgctgctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtg

SEQ ID NO: 57 - DNA, 1437 bp, Artificial Sequence
  Full 16sRNA of Clostridium paraputrificum
gacgaacgctggcggcgtgcctaacacatgcaagtcgagcgatgaagttccttcgggaacggattagcggcggacgggtgagtaacacgtgggcaacctgccttatagaggggaatagccttccgaaaggaagattaataccgcataagattgtagcttcgcatgaagtagcaattaaaggagcaatccgctataagatgggcccgcggcgcattagctagttggtgaggtaacggctcaccaaggcgacgatgcgtagccgacctgagagggtgatcggccacattgggactgagacacggcccagactcctacgggaggcagcagtggggaatattgcacaatgggggaaaccctgatgcagcaacgccgcgtgagtgatgacggccttcgggttgtaaagctctgtctttggggacgataatgacggtacccaaggaggaagccacggctaactacgtgccagcagccgcggtaatacgtaggtggcaagcgttgtccggatttactgggcgtaaagggagcgtaggcggatttttaagtgggatgtgaaatacccgggctcaacctgggtgctgcattccaaactggaaatctagagtgcaggaggggaaagtggaattcctagtgtagcggtgaaatgcgtagagattaggaagaacaccagtggcgaaggcgactttctggactgtaactgacgctgaggctcgaaagcgtggggagcaaacaggattagataccctggtagtccacgccgtaaacgatgaatactaggtgtaggggttgtcatgacctctgtgccgccgctaacgcattaagtattccgcctggggagtacggtcgcaagattaaaactcaaaggaattgacgggggcccgcacaagtagcggagcatgtggtttaattcgaagcaacgcgaagaaccttacctagacttgacatctcctgaattaccatgtaatgtgggaagtcctttcggggacaggaagacaggtggtgcatggttgtcgtcagctcgtgtcgtgagatgttgggttaagtcccgcaacgagcgcaacccttattgttagttgctaccatttagttgagcactctagcgagactgcccgggttaaccgggaggaaggtggggatgacgtcaaatcatcatgccccttatgtctagggctacacacgtgctacaatggccggtacaacgagatgcaataccgtgaggtggagcaaaactataaaaccggtctcagttcggattgtaggctgaaactcgcctacatgaagctggagttactagtaatcgcgaatcagaatgtcgcggtgaatacgttcccgggccttgtacacaccgcccgtcacaccatgagagttggcaatacccaaagttggtgatctaacccgtaagggaggaagccacctaaggtagggtcagcgattggggtg

SEQ ID NO: 58 - DNA, 1450 bp, Artificial Sequence
  Full 16sRNA of Bacteroides vulgatus
gatgaacgctagctacaggcttaacacatgcaagtcgaggggcagcatggtcttagcttgctaaggccgatggcgaccggcgcacgggtgagtaacacgtatccaacctgccgtctactcttggacagccttctgaaaggaagattaatacaagatggcatcatgagtccgcatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcgagagcctgaaccagccaagtagcgtgaaggatgactgccctatgggttgtaaacttcttttataaaggaataaagtcgggtatgcatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaggaactccgattgcgaaggcagcctgctaagctgcaactgacattgaggctcgaaagtgtgggtatcaaacaggattagataccctggtagtccacacggtaaacgatgaatactcgctgtttgcgatatacggcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggttctgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatcccaaaaacctctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgaggagcgccctagggtaaaactggtgactggggct

SEQ ID NO: 59 - DNA, 1485 bp, Artificial Sequence
  Full 16sRNA of Veillonella atypica
gacgaacgctggcggcgtgcttaacacatgcaagtcgaacgaagagcgatggaagcttgcttctatcaatcttagtggcgaacgggtgagtaacgcgtaatcaacctgcccttcagagggggacaacagttggaaacgactgctaataccgcatacgatccaatctcggcatcgagactggatgaaaggtggcctctatttataagctatcactgaaggaggggattgcgtctgattagctagttggaggggtaacggcccaccaaggcgatgatcagtagccggtctgagaggatgaacggccacattgggactgagacacggcccagactcctacgggaggcagcagtggggaatcttccgcaatggacgaaagtctgacggagcaacgccgcgtgagtgatgacggccttcgggttgtaaagctctgttaatcgggacgaatggttcttgtgcgaatagtgcgaggatttgacggtaccggaatagaaagccacggctaactacgtgccagcagccgcggtaatacgtaggtggcaagcgttgtccggaattattgggcgtaaagcgcgcgcaggcggatcagttagtctgtcttaaaagttcggggcttaaccccgtgatgggatggaaactgctgatctagagtatcggagaggaaagtggaattcctagtgtagcggtgaaatgcgtagatattaggaagaacaccagtggcgaaggcgactttctggacgaaaactgacgctgaggcgcgaaagccaggggagcgaacgggattagataccccggtagtcctggccgtaaacgatgggtactaggtgtaggaggtatcgaccccttctgtgccggagttaacgcaataagtaccccgcctggggagtacgaccgcaaggttgaaactcaaaggaattgacgggggcccgcacaagcggtggagtatgtggtttaattcgacgcaacgcgaagaaccttaccaggtcttgacattgatggacagaaccagagatggttcctcttcttcggaagccagaaaacaggtggtgcacggttgtcgtcagctcgtgtcgtgagatgttgggttaagtcccgcaacgagcgcaacccctatcttatgttgccagcacttcgggtgggaactcatgagagactgccgcagacaatgcggaggaaggcggggatgacgtcaaatcatcatgccccttatgacctgggctacacacgtactacaatgggagttaatagacggaagcgaaaccgcgaggtggagcaaacccgagaaacactctctcagttcggatcgtaggctgcaactcgcctacgtgaagtcggaatcgctagtaatcgcaggtcagcatactgcggtgaatacgttcccgggccttgtacacaccgcccgtcacaccacgaaagtcggaagtgcccaaagccggtggggtaaccttcgggagccagccgtctaaggtaaagtcgatgattggggtg

SEQ ID NO: 60 - DNA, 1485 bp, Artificial Sequence
  Full 16sRNA of Veillonella dispar
gacgaacgctggcggcgtgcttaacacatgcaagtcgaacgaagagcgatggaagcttgcttctatcaatcttagtggcgaacgggtgagtaacgcgtaatcaacctgcccttcagagggggacaacagttggaaacgactgctaataccgcatacgatctaacctcggcatcgaggatagatgaaaggtggcctctatttataagctatcactgaaggaggggattgcgtctgattagctagttggaggggtaacggcccaccaaggcgatgatcagtagccggtctgagaggatgaacggccacattgggactgagacacggcccagactcctacgggaggcagcagtggggaatcttccgcaatggacgaaagtctgacggagcaacgccgcgtgagtgatgacggccttcgggttgtaaagctctgttaatcgggacgaaaggccttcttgcgaatagttagaaggattgacggtaccggaatagaaagccacggctaactacgtgccagcagccgcggtaatacgtaggtggcaagcgttgtccggaattattgggcgtaaagcgcgcgcaggcggattggtcagtctgtcttaaaagttcggggcttaaccccgtgatgggatggaaactgccaatctagagtatcggagaggaaagtggaattcctagtgtagcggtgaaatgcgtagatattaggaagaacaccagtggcgaaggcgactttctggacgaaaactgacgctgaggcgcgaaagccaggggagcgaacgggattagataccccggtagtcctggccgtaaacgatgggtactaggtgtaggaggtatcgaccccttctgtgccggagttaacgcaataagtaccccgcctggggagtacgaccgcaaggttgaaactcaaaggaattgacgggggcccgcacaagcggtggagtatgtggtttaattcgacgcaacgcgaagaaccttaccaggtcttgacattgatggacagaactagagatagttcctcttcttcggaagccagaaaacaggtggtgcacggttgtcgtcagctcgtgtcgtgagatgttgggttaagtcccgcaacgagcgcaacccctatcttatgttgccagcactttgggtgggaactcatgagagactgccgcagacaatgcggaggaaggcggggatgacgtcaaatcatcatgccccttatgacctgggctacacacgtactacaatgggagttaatagacggaagcaataccgcgaggtggagcaaacccgagaaacactctctcagttcggatcgtaggctgcaactcgcctacgtgaagtcggaatcgctagtaatcgcaggtcagcatactgcggtgaatacgttcccgggccttgtacacaccgcccgtcacaccacgaaagtcggaagtgcccaaagccggtggggtaaccttcgggagccagccgtctaaggtaaagtcgatgattggggtg

SEQ ID NO: 61 - DNA, 1453 bp, Artificial Sequence
  Full 16sRNA of Pseudoflavonifractor
gatgaacgctggcggcgtgcttaacacatgcaagtcgaacggagagctcatgacagaggattcgtccaatggattgggtttcttagtggcggacgggtgagtaacgcgtgaggaacctgcctcggagtggggaataacagtccgaaaggactgctaataccgcataatgcagctgagtcgcatgacctggctgccaaagatttatcgctctgagatggcctcgcgtctgattagctagttggcggggtaacggcccaccaaggcgacgatcagtagccggactgagaggttggccggccacattgggactgagacacggcccagactcctacgggaggcagcagtggggaatattgggcaatgggcgcaagcctgacccagcaacgccgcgtgaaggatgaaggctttcgggttgtaaacttcttttatcagggacgaaataaatgacggtacctgatgaataagccacggctaactacgtgccagcagccgcggtaatacgtaggtggcaagcgttatccggatttactgggtgtaaagggcgtgtaggcgggactgcaagtcaggtgtgaaaaccacgggctcaacctgtggcctgcatttgaaactgtagttcttgagtgctggagaggcaatcggaattccgtgtgtagcggtgaaatgcgtagatatacggaggaacaccagtggcgaaggcggattgctggacagtaactgacgctgaggcgcgaaagcgtggggagcaaacaggattagataccctggtagtccacgccgtaaacgatggatactaggtgtggggggactgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatccgactaacgaagcagagatgcattaggtgcccttcggggaaagtcgagacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttatgtcctgggccacacacgtactacaatggtggttaacagagggaagcaatgccgcgaggtggagcaaatccctaaaagccatcccagttcggattgcaggctgaaacccgcctgtatgaagttggaatcgctagtaatcgcggatcagcatgccgcggtgaatacgttcccgggccttgtacacaccgcccgtcacaccatgagagtcgggaacacccgaagtccgtagcctaaccgcaaggagggcgcggccgaaggtgggttcgataattggggtg

SEQ ID NO: 62 - DNA, 1425 bp, Artificial Sequence; identical to SEQ ID NO: 54
  Full 16sRNA of Clostridioides

SEQ ID NO: 63 - DNA, 1464 bp, Artificial Sequence; identical to SEQ ID NO: 56
  Full 16sRNA of Escherichia

SEQ ID NO: 64 - DNA, 1449 bp, Artificial Sequence
  Full 16sRNA of Clostridium_g24
  misc_feature (508): n is a, g, c or t
  misc_feature (891): n is a, g, c or t
  misc_feature (928): n is a, g, c or t
  misc_feature (984): n is a, g, c or t
  misc_feature (1427): n is a, g, c or t
  misc_feature (1445): n is a, g, c or t
gatcaacgctggcggcgtgcctaacacatgcaagtcgaacgaagcaattaagatgaagttttcggatggaatcttgattgactgagtggcggacgggtgagtaacgcgtggataacctgcctcacactgggggataacagttagaaatgactgctaataccgcataagcgcacagtgccgcatggcagtgtgtgaaaaactccggtagtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcnagcgttatccggatttactgggtgtaaagggagcgtagacggcgaagcaagtctgaagtgaaaacccagggctcaaccctggcactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagccttcggtgccgccgcaaacgcagtaagcattccacctggggagtacgttcgcaagaatgaaactcaaaggaattgacgggganccgcacaagcggtggagcatgtggtttaattcgaagnaacgcgaagaaccttaccaagtcttgacatccccctgacggccggtaacgcggccnttcttcgggacaggggagacaggtggtgcatggttgtcgtcagctcgtgtcgtgagatgttgggttaagtcccgcaacgagcgcaacccttatccttagtagccagcaggtaaagctgggcactctagggagactgccagggataacctggaggaaggtggggatgacgtcaaatcatcatgccccttatgatttgggctacacacgtgctacaatggcgtaaacaaagggaagcgagacagtgatgtggagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaaccgaaaggagggagctgccnaaggcggggcaggtaacngggg

SEQ ID NO: 65 - DNA, 1435 bp, Artificial Sequence
  Full 16sRNA of Clostridium
gacgaacgctggcggcgtgcttaacacatgcaagtcgagcgatgaagctccttcgggagtggattagcggcggacgggtgagtaacacgtgggtaacctgcctcatagaggggaatagcctttcgaaaggaagattaataccgcataagattgtagtaccgcatggtacagcaattaaaggagtaatccgctatgagatggacccgcgtcgcattagctagttggtgaggtaacggctcaccaaggcgacgatgcgtagccgacctgagagggtgatcggccacattgggactgagacacggcccagactcctacgggaggcagcagtggggaatattgcacaatgggggaaaccctgatgcagcaacgccgcgtgagtgatgacggtcttcggattgtaaagctctgtctttagggacgataatgacggtacctaaggaggaagccacggctaactacgtgccagcagccgcggtaatacgtaggtggcaagcgttgtccggatttactgggcgtaaagggagcgtaggtggatatttaagtgggatgtgaaatacccgggcttaacctgggtgctgcattccaaactggatatctagagtgcaggagaggaaaggagaattcctagtgtagcggtgaaatgcgtagagattaggaagaataccagtggcgaaggcgactttctggactgtaactgacactgaggctcgaaagcgtggggagcaaacaggattagataccctggtagtccacgccgtaaacgatgaatactaggtgtaggggttgtcatgacctctgtgccgccgctaacgcattaagtattccgcctggggagtacggtcgcaagattaaaactcaaaggaattgacgggggcccgcacaagcagcggagcatgtggtttaattcgaagcaacgcgaagaaccttacctagacttgacatctcctgaattactctgtaatggaggaagccacttcggtggcaggaagacaggtggtgcatggttgtcgtcagctcgtgtcgtgagatgttgggttaagtcccgcaacgagcgcaacccttattgttagttgctaccatttagttgagcactctagcgagactgcccgggttaaccgggaggaaggtggggatgacgtcaaatcatcatgccccttatgtctagggctacacacgtgctacaatggtcggtacaatgagatgcaacctcgcgagagtgagcaaaactataaaaccgatctcagttcggattgtaggctgaaactcgcctacatgaagctggagttgctagtaatcgcgaatcagaatgtcgcggtgaatacgttcccgggccttgtacacaccgcccgtcacaccatgagagttggcaatacccaaagttcgtgagctaaccgcaaggaggcagcgacctaaggtagggtcagcgattggggtg

SEQ ID NO: 66 - DNA, 1485 bp, Artificial Sequence
  Full 16sRNA of Veillonella
gacgaacgctggcggcgtgcttaacacatgcaagtcgaacgaagagcgatggaagcttgcttctatcaatcttagtggcgaacgggtgagtaacgcgtaatcaacctgcccttcagagggggacaacagttggaaacgactgctaataccgcatacgatctaacctcggcatcgaggaaagatgaaaggtggcctctatttataagctatcactgaaggaggggattgcgtctgattagctagttggaggggtaacggcccaccaaggcgatgatcagtagccggtctgagaggatgaacggccacattgggactgagacacggcccagactcctacgggaggcagcagtggggaatcttccgcaatggacgaaagtctgacggagcaacgccgcgtgagtgatgacggccttcgggttgtaaagctctgttaatcgggacgaaaggccttcttgcgaacagttagaaggattgacggtaccggaatagaaagccacggctaactacgtgccagcagccgcggtaatacgtaggtggcaagcgttgtccggaattattgggcgtaaagcgcgcgcaggcggatcagtcagtctgtcttaaaagttcggggcttaaccccgtgatgggatggaaactgctgatctagagtatcggagaggaaagtggaattcctagtgtagcggtgaaatgcgtagatattaggaagaacaccagtggcgaaggcgactttctggacgaaaactgacgctgaggcgcgaaagccaggggagcgaacgggattagataccccggtagtcctggccgtaaacgatgggtactaggtgtaggaggtatcgaccccttctgtgccggagttaacgcaataagtaccccgcctggggagtacgaccgcaaggttgaaactcaaaggaattgacgggggcccgcacaagcggtggagtatgtggtttaattcgacgcaacgcgaagaaccttaccaggtcttgacattgatggacagaaccagagatggttcctcttcttcggaagccagaaaacaggtggtgcacggttgtcgtcagctcgtgtcgtgagatgttgggttaagtcccgcaacgagcgcaacccctatcttatgttgccagcactttgggtgggaactcatgagagactgccgcagacaatgcggaggaaggcggggatgacgtcaaatcatcatgccccttatgacctgggctacacacgtactacaatgggagttaatagacggaagcgagatcgcgagatggagcaaacccgagaaacactctctcagttcggatcgtaggctgcaactcgcctacgtgaagtcggaatcgctagtaatcgcaggtcagcatactgcggtgaatacgttcccgggccttgtacacaccgcccgtcacaccacgaaagtcggaagtgcccaaagccggtggggtaaccttcgggagccagccgtctaaggtaaagtcgatgattggggtg

SEQ ID NO: 67 - DNA, 1454 bp, Artificial Sequence
  Full 16sRNA of Bacteroides dorei
gatgaacgctagctacaggcttaacacatgcaagtcgaggggcagcatggtcttagcttgctaaggctgatggcgaccggcgcacgggtgagtaacacgtatccaacctgccgtctactcttggccagccttctgaaaggaagattaatccaggatgggatcatgagttcacatgtccgcatgattaaaggtattttccggtagacgatggggatgcgttccattagatagtaggcggggtaacggcccacctagtcaacgatggataggggttctgagaggaaggtcccccacattggaactgagacacggtccaaactcctacgggaggcagcagtgaggaatattggtcaatgggcgatggcctgaaccagccaagtagcgtgaaggatgactgccctatgggttgtaaacttcttttataaaggaataaagtcgggtatgcatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatgtcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatacggcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcactcgaatgatccggaaacggttcagctagcaatagcgagtgtgaaggtgctgcatggttgtcgtcagctcgtgccgtgaggtgtcggcttaagtgccataacgagcgcaacccttgttgtcagttactaacaggtgatgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatccctaaaacccctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgaggatcgccctagggtaaaactggtgactggggct

SEQ ID NO: 68 - DNA, 1452 bp, Artificial Sequence
  Full 16sRNA of PAC001148_s
gatgaacgctggcggcgtgcctaacacatgcaagtcgagcgaagcggtctggaggaagttttcggatggaatccggattgactgagcggcggacgggtgagtaacgcgtgggtaacctgcctcatacagggggataacagttagaaatggctgctaataccgcataagcgcacagcttcgcatggagcagtgtgaaaaactccggtggtatgagatggacccgcgtctgattagctagttggtaaggtaacggcttaccaaggcgacgatcagtagccgacctgagagggtgaccggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatagcaagtctggagtgaaagcccggggctcaaccccggtactgctttggaaactgttaagctagagtgctggagaggtaagtggaattcctagtgtagcggtgaaatgcgtagatattaggaggaacaccagtggcgaaggcggcttactggacagtaactgacgttgaggctcgaaagcgtggggagcaaacaggattagataccctggtagtccacgccgtaaacgatgaatactaggtgttggtgggcaaagcccatcggtgccgccgcaaacgcaataagtattccacctggggagtacgttcgcaagaatgaaactcaaaggaattgacggggacccgcacaagcggtggagtatgtggtttaattcgaagcaacgcgaagaaccttaccaagtcttgacatcggaatgaccgggaagtaatgttcccttctctacggagcattccagacaggtggtgcatggttgtcgtcagctcgtgtcgtgagatgttgggttaagtcccgcaacgagcgcaacccttatccttagtagccagcagtaagatgggcactctagggagactgccagggataacctggaggaaggtggggatgacgtcaaatcatcatgccccttatgatttgggctacacacgtgctacaatggcgtaaacaaagagaggcgagcctgcgagggggagcgaatctcaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgactcaaccgaaaggggagagctgccgaaggcggggcaggtaactggggtg

SEQ ID NO: 69 - DNA, 1462 bp, Artificial Sequence
  Full 16sRNA of Haemophilus parainfluenzae group
attgaacgctggcggcaggcttaacacatgcaagtcgaacggtaacatgaagaagcttgcttctttgatgacgagtggcggacgggtgagtaatgcttgggaatctagcttatggagggggataactacgggaaactgtagctaataccgcgtagtatcggaagatgaaagtgtgggaccttcgggccacatgccataggatgagcccaagtgggattaggtagttggtgaggtaaaggctcaccaagccgacgatctctagctggtctgagaggatgaccagccacactgggactgagacacggcccagactcctacgggaggcagcagtggggaatattgcgcaatgggggcaaccctgacgcagccatgccgcgtgaatgaagaaggccttcgggttgtaaagttctttcggtagcgaggaaggcatttagtttaatagactaggtgattgacgttaactacagaagaagcaccggctaactccgtgccagcagccgcggtaatacggagggtgcgagcgttaatcggaataactgggcgtaaagggcacgcaggcggtgacttaagtgaggtgtgaaagccccgggcttaacctgggaattgcatttcatactgggtcgctagagtactttagggaggggtagaattccacgtgtagcggtgaaatgcgtagagatgtggaggaataccgaaggcgaaggcagccccttgggaatgtactgacgctcatgtgcgaaagcgtggggagcaaacaggattagataccctggtagtccacgctgtaaacgatgtcgatttgggggttgagctttaagcttggcgcccgtagctaacgtgataaatcgaccgcctggggagtacggccgcaaggttaaaactcaaatgaattgacgggggcccgcacaagcggtggagcatgtggtttaattcgatgcaacgcgaagaaccttacctactcttgacatccagagaacattccagagatggattggtgccttcgggaactctgagacaggtgctgcatggctgtcgtcagctcgtgttgtgaaatgttgggttaagtcccgcaacgagcgcaacccttatcctttgttgccagcgattcggtcgggaactcaaaggagactgccggtgataaaccggaggaaggtggggatgacgtcaagtcatcatggcccttacgagtagggctacacacgtgctacaatggcgtatacagagggaagcgagagtgcgagctggagcgaatctcacaaagtacgtctaagtccggattggagtctgcaactcgactccatgaagtcggaatcgctagtaatcgcaaatcagaatgttgcggtgaatacgttcccgggccttgtacacaccgcccgtcacaccatgggagtgggttgtaccagaagtagatagcttaaccttcgggggggcgtttaccacggtatgattcatgactggggtg

SEQ ID NO: 70 - DNA, 1492 bp, Artificial Sequence
  Full 16sRNA of Lactobacillus paracasei group
gatgaacgctggcggcgtgcctaatacatgcaagtcgaacgagttctcgttgatgatcggtgcttgcaccgagattcaacatggaacgagtggcggacgggtgagtaacacgtgggtaacctgcccttaagtgggggataacatttggaaacagatgctaataccgcatagatccaagaaccgcatggttcttggctgaaagatggcgtaagctatcgcttttggatggacccgcggcgtattagctagttggtgaggtaacggctcaccaaggcgatgatacgtagccgaactgagaggttgatcggccacattgggactgagacacggcccaaactcctacgggaggcagcagtagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagcgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcatttagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccggtggcgtaacccttttagggagcgagccgtctaaggtgggacaaatgattagggtg

SEQ ID NO: 71 - DNA, 1451 bp, Artificial Sequence
  Full 16sRNA of Bacteroides ovatus group
gatgaacgctagctacaggcttaacacatgcaagtcgaggggcagcattttagtttgcttgcaaactgaagatggcgaccggcgcacgggtgagtaacacgtatccaacctgccgataactccggaatagcctttcgaaagaaagattaataccggatagcatacgaatatcgcatgatatttttattaaagaatttcggttatcgatggggatgcgttccattagtttgttggcggggtaacggcccaccaagactacgatggataggggttctgagaggaaggtcccccacattggaactgagacacggtccaaactcctacgggaggcagcagtgaggaatattggtcaatgggcgagagcctgaaccagccaagtagcgtgaaggatgaaggctctatgggtcgtaaacttcttttatatgggaataaagttttccacgtgtggaattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgttactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaacagaatatattggaaacagtatagccgtaaggctgttgtgaaggtgctgcatggttgtcgtcagctcgtgccgtgaggtgtcggcttaagtgccataacgagcgcaacccttatctttagttactaacaggttatgctgaggactctagagagactgccgtcgtaagatgtgaggaaggtggggatgacgtcaaatcagcacggcccttacgtccggggctacacacgtgttacaatggggggtacagaaggcagctacacggcgacgtgatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagcgtcctagggtaaaactggtaattggggct

SEQ ID NO: 72 - DNA, 1498 bp, Artificial Sequence
  Full 16sRNA of Lactobacillus fermentum
gatgaacgccggcggtgtgcctaatacatgcaagtcgaacgcgttggcccaattgattgacggtgcttgcacctgattgattttggtcgccaacgagtggcggacgggtgagtaacacgtaggtaacctgcccagaagcgggggacaacatttggaaacagatgctaataccgcataacagcgttgttcgcatgaacaacgcttaaaagatggcttctcgctatcacttctggatggacctgcggtgcattagcttgttggtggggtaacggcctaccaaggcgatgatgcatagccgagttgagagactgatcggccacaatgggactgagacacggcccatactcctacgggaggcagcagtagggaatcttccacaatgggcgcaagcctgatggagcaacaccgcgtgagtgaagaagggtttcggctcgtaaagctctgttgttaaagaagaacacgtatgagagtaactgttcatacgttgacggtatttaaccagaaagtcacggctaactacgtgccagcagccgcggtaatacgtaggtggcaagcgttatccggatttattgggcgtaaagagagtgcaggcggttttctaagtctgatgtgaaagccttcggcttaaccggagaagtgcatcggaaactggataacttgagtgcagaagagggtagtggaactccatgtgtagcggtggaatgcgtagatatatggaagaacaccagtggcgaaggcggctacctggtctgcaactgacgctgagactcgaaagcatgggtagcgaacaggattagataccctggtagtccatgccgtaaacgatgagtgctaggtgttggagggtttccgcccttcagtgccggagctaacgcattaagcactccgcctggggagtacgaccgcaaggttgaaactcaaaggaattgacgggggcccgcacaagcggtggagcatgtggtttaattcgaagctacgcgaagaaccttaccaggtcttgacatcttgcgccaaccctagagatagggcgtttccttcgggaacgcaatgacaggtggtgcatggtcgtcgtcagctcgtgtcgtgagatgttgggttaagtcccgcaacgagcgcaacccttgttactagttgccagcattaagttgggcactctagtgagactgccggtgacaaaccggaggaaggtggggacgacgtcagatcatcatgccccttatgacctgggctacacacgtgctacaatggacggtacaacgagtcgcgaactcgcgagggcaagcaaatctcttaaaaccgttctcagttcggactgcaggctgcaactcgcctgcacgaagtcggaatcgctagtaatcgcggatcagcatgccgcggtgaatacgttcccgggccttgtacacaccgcccgtcacaccatgagagtttgtaacacccaaagtcggtggggtaaccttttaggagccagccgcctaaggtgggacagatgattagggtg

SEQ ID NO: 73 - DNA, 1453 bp, Artificial Sequence
  Full 16sRNA of Clostridium_g35
gatgaacgctggcggcgtgcctaacacatgcaagtcgaacgaagcgatttaacggaagttttcggatggaagttgaattgactgagtggcggacgggtgagtaacgcgtgggtaacctgccttgtactgggggacaacagttagaaatgactgctaataccgcataagcgcacagtattgcatgatacagtgtgaaaaactccggtggtacaagatggacccgcgtctgattagctagttggtaaggtaacggcttaccaaggcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggtaaagcaagtctgaagtgaaagcccgcggctcaactgcgggactgctttggaaactgtttaactggagtgtcggagaggtaagtggaattcctagtgtagcggtgaaatgcgtagatattaggaggaacaccagtggcgaaggcgacttactggacgataactgacgttgaggctcgaaagcgtggggagcaaacaggattagataccctggtagtccacgccgtaaacgatgaatactaggtgttggggagcaaagctcttcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaggtcttgacatcgatccgacgggggagtaacgtccccttcccttcggggcggagaagacaggtggtgcatggttgtcgtcagctcgtgtcgtgagatgttgggttaagtcccgcaacgagcgcaacccttattctaagtagccagcggttcggccgggaactcttgggagactgccagggataacctggaggaaggtggggatgacgtcaaatcatcatgccccttatgatctgggctacacacgtgctacaatggcgtaaacaaagagaagcaagaccgcgaggtggagcaaatctcaaaaataacgtctcagttcggactgcaggctgcaactcgcctgcacgaagctggaatcgctagtaatcgcgaatcagaatgtcgcggtgaatacgttcccgggtcttgtacacaccgcccgtcacaccatgggagtcagtaacgcccgaagtcagtgacccaaccgcaaggagggagctgccgaaggcgggaccgataactggggtg

SEQ ID NO: 74 - DNA, 1425 bp, Artificial Sequence
  Full 16sRNA of Intestinibacter
gatgaacgctggcggcgtgcctaacacatgcaagtcgagcgattctcttcggagaagagcggcggacgggtgagtaacgcgtgggtaacctgccctgtacacacggataacataccgaaaggtatgctaatacgggataacataagaaattcgcatgtttttcttatcaaagctccggcggtacaggatggacccgcgtctgattagctagttggtgaggtaacggctcaccaaggcgacgatcagtagccgacctgagagggtgatcggccacattggaactgagacacggtccaaactcctacgggaggcagcagtggggaatattgcacaatgggcgaaagcctgatgcagcaacgccgcgtgagcgatgaaggccttcgggtcgtaaagctctgtcctcaaggaagataatgacggtacttgaggaggaagccccggctaactacgtgccagcagccgcggtaatacgtagggggctagcgttatccggatttactgggcgtaaagggtgcgtaggcggtcttttaagtcaggagtgaaaggctacggctcaaccgtagtaagctcttgaaactggaggacttgagtgcaggagaggagagtggaattcctagtgtagcggtgaaatgcgtagatattaggaggaacaccagtagcgaaggcggctctctggactgtaactgacgctgaggcacgaaagcgtggggagcaaacaggattagataccctggtagtccacgccgtaaacgatgagtactaggtgtcgggggttacccccctcggtgccgcagctaacgcattaagtactccgcctggggagtacgctcgcaagagtgaaactcaaaggaattgacggggacccgcacaagtagcggagcatgtggtttaattcgaagcaacgcgaagaaccttacctaagcttgacatccttttgaccgatgcctaatcgcatctttcccttcggggacagaagtgacaggtggtgcatggttgtcgtcagctcgtgtcgtgagatgttgggttaagtcccgcaacgagcgcaacccttgcctttagttgccatcattaagttgggcactctagagggactgccagggataacctggaggaaggtggggatgacgtcaaatcatcatgccccttatgcttagggctacacacgtgctacaatgggtggtacagagggcagcgaagtcgtgaggccaagctaatcccttaaagccattctcagttcggattgtaggctgaaactcgcctacatgaagctggagttactagtaatcgcagatcagaatgctgcggtgaatgcgttcccgggtcttgtacacaccgcccgtcacaccatgggagttgggggcgcccgaagccggctagctaaccttttggaagcggtcgtcgaaggtgaaaccaataactggggtg

SEQ ID NO: 75 - DNA, 1409 bp, Artificial Sequence
  Full 16sRNA of Hungatella
atgcagtcgagcgaagcgattctctaggaagttttcggatggaataggatttgacttagcggcggacgggtgagtaacgcgtgggtaacctgccttacactgggggataacagttagaaatgactgctaataccgcataagcgcacagggccgcatggtctggtgtgaaaaactccggtggtgtaagatggacccgcgtctgattaggtagttggtggggtaacggcccaccaagccgacgatcagtagccgacctgagagggtgaccggccacattgggactgagacacggcccaaactcctacgggaggcagcagtggggaatattggacaatgggcgaaagcctgatccagcgacgccgcgtgagtgaagaagtgtttcggcatgtaaagctctatcagcagggaagaaaatgacggtacctgactaagaagccccggctaactacgtgccagcagccgcggtaatacgtagggggcaagcgttatccggatttactgggtgtaaagggagcgtagacggttaagcaagtctgaagtgaaagcccggggctcaaccccggtactgctttggaaactgtttgacttgagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatactaggtgtcgggggacaaagtccttcggtgccgccgctaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccattgaaaatcatttaaccggtatccctcttcggagcaatggagacaggtggtgcatggttgtcgtcagctcgtgtcgtgagatgttgggttaagtcccgcaacgagcgcaacccttatccttagtagccagcacataatggtgggcactctggggagactgccagggataacctggaggaaggtggggatgacgtcaaatcatcatgccccttatgatttgggctacacacgtgctacaatggcgtaaacaaagggaagcaaaggagcgatctggagcaaaccccaaaaataacgtctcagttcggattgcaggctgcaactcgcctgcatgaagctggaatcgctagtaatcgcgaatcagaatgtcgcggtgaatacgttcccgggtcttgtacacaccgcccgtcacaccatgggagttggtaacgcccgaagtcagtgacccaaccgcaaggagggagctgccgaaggcg

SEQ ID NO: 76 - DNA, 1453 bp, Artificial Sequence
  Full 16sRNA of Prevotella
gatgaacgctagctacaggcttaacacatgcaagtcgaggggaaacggcattgagtgcttgcactctttggacgtcgaccggcgcacgggtgagtaacgcgtatccaaccttcccattactgtgggataacctgccgaaaggcagactaataccgcatagtcttcgatgacggcatcagatttgaagtaaagatttatcggtaatggatggggatgcgtctgattagcttgttggcggggtaacggcccaccaaggcaacgatcagtaggggttctgagaggaaggtcccccacattggaactgagacacggtccaaactcctacgggaggcagcagtgaggaatattggtcaatggacggaagtctgaaccagccaagtagcgtgcaggatgacggccctatgggttgtaaactgcttttgtatggggataaagttagggacgtgtccctatttgcaggtaccatacgaataaggaccggctaattccgtgccagcagccgcggtaatacggaaggtccaggcgttatccggatttattgggtttaaagggagcgtaggctggagattaagtgtgttgtgaaatgtagacgctcaacgtctgaattgcagcgcatactggtttccttgagtacgcacaacgttggcggaattcgtcgtgtagcggtgaaatgcttagatatgacgaagaactccgattgcgaaggcagctgacgggagcgcaactgacgcttaagctcgaaggtgcgggtatcaaacaggattagataccctggtagtccgcacagtaaacgatggatgcccgctgttggtacctggtatcagcggctaagcgaaagcattaagcatcccacctggggagtacgccggcaacggtgaaactcaaaggaattgacgggggcccgcacaagcggaggaacatgtggtttaattcgatgatacgcgaggaaccttacccgggcttgaattgcagaggaaggatttagagataatgacgcccttcggggtctctgtgaaggtgctgcatggttgtcgtcagctcgtgccgtgaggtgtcggcttaagtgccataacgagcgcaacccctctcttcagttgccatcaggttaagctgggcactctggagacactgccaccgtaaggtgtgaggaaggtggggatgacgtcaaatcagcacggcccttacgtccggggctacacacgtgttacaatggccggtacagagggacggtgtaatgcaaattgcatccaatcttgaaagccggtcccagttcggactggggtctgcaacccgaccccacgaagctggattcgctagtaatcgcgcatcagccatggcgcggtgaatacgttcccgggccttgtacacaccgcccgtcaagccatgaaagccgggggtgcctgaagtccgtgaccgcaaggatcggcctagggcaaaactggtgattggggct

SEQ ID NO: 77 - DNA, 1471 bp, Artificial Sequence
  Full 16sRNA of Streptococcus
gacgaacgctggcggcgtgcctaatacatgcaagtagaacgctgagaactggtgcttgcaccggttcaaggagttgcgaacgggtgagtaacgcgtaggtaacctacctcatagcgggggataactattggaaacgatagctaataccgcataagagagactaacgcatgttagtaatttaaaaggggcaattgctccactatgagatggacctgcgttgtattagctagttggtgaggtaaaggctcaccaaggcgacgatacatagccgacctgagagggtgatcggccacactgggactgagacacggcccagactcctacgggaggcagcagtagggaatcttcggcaatgggggcaaccctgaccgagcaacgccgcgtgagtgaagaaggttttcggatcgtaaagctctgttgttagagaagaatgatggtgggagtggaaaatccaccaagtgacggtaactaaccagaaagggacggctaactacgtgccagcagccgcggtaatacgtaggtcccgagcgttgtccggatttattgggcgtaaagcgagcgcaggcggttttttaagtctgaagttaaaggcattggctcaaccaatgtacgctttggaaactggagaacttgagtgcagaaggggagagtggaattccatgtgtagcggtgaaatgcgtagatatatggaggaacaccggtggcgaaagcggctctctggtctgtaactgacgctgaggctcgaaagcgtggggagcaaacaggattagataccctggtagtccacgccgtaaacgatgagtgctaggtgttaggccctttccggggcttagtgccggagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcccgatgcccgctctagagatagagttttacttcggtacatcggtgacaggtggtgcatggttgtcgtcagctcgtgtcgtgagatgttgggttaagtcccgcaacgagcgcaacccctattgttagttgccatcattaagttgggcactctagcgagactgccggtaataaaccggaggaaggtggggatgacgtcaaatcatcatgccccttatgacctgggctacacacgtgctacaatggttggtacaacgagtcgcaagccggtgacggcaagctaatctcttaaagccaatctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaacctattaggagccagccgcctaaggtgggatagatgattggggtg

SEQ ID NO: 78 - DNA, 1464 bp, Artificial Sequence
  Full 16sRNA of Citrobacter
attgaacgctggcggcaggcctaacacatgcaagtcgaacggtagcacagaggagcttgctccttgggtgacgagtggcggacgggtgagtaatgtctgggaaactgcccgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcgaggaggaaggcgttgtggttaataaccgcagcgattgacgttactcgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcatccgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaacttagcagagatgctttggtgccttcgggaactctgagacaggtgctgcatggctgtcgtcagctcgtgttgtgaaatgttgggttaagtcccgcaacgagcgcaacccttatcctttgttgccagcggttcggccgggaactcaaaggagactgccagtgataaactggaggaaggtggggatgacgtcaagtcatcatggcccttacgagtagggctacacacgtgctacaatggcatatacaaagagaagcgacctcgcgagagcaagcggacctcataaagtatgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtg

SEQ ID NO: 79 - DNA, 1462 bp, Artificial Sequence
  Full 16sRNA of Klebsiella
attgaacgctggcggcaggcctaacacatgcaagtcgagcggtagcacagagagcttgctctcgggtgacgagcggcggacgggtgagtaatgtctgggaaactgcctgatggagggggataactactggaaacggtagctaataccgcataatgtcgcaagaccaaagtgggggaccttcgggcctcatgccatcagatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcggggaggaaggcgatgaggttaataacctcatcgattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgatttggaggttgtgcccttgaggcgtggcttccggagctaacgcgttaaatcgaccgcctggggagtacggccgcaaggttaaaactcaaatgaattgacgggggcccgcacaagcggtggagcatgtggtttaattcgatgcaacgcgaagaaccttacctggtcttgacatccacagaactttccagagatggattggtgccttcgggaactgtgagacaggtgctgcatggctgtcgtcagctcgtgttgtgaaatgttgggttaagtcccgcaacgagcgcaacccttatcctttgttgccagcggttaggccgggaactcaaaggagactgccagtgataaactggaggaaggtggggatgacgtcaagtcatcatggcccttacgaccagggctacacacgtgctacaatggcatatacaaagagaagcgacctcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtg

SEQ ID NO: 80 - DNA, 1462 bp, Artificial Sequence
  Full 16sRNA of Haemophilus
attgaacgctggcggcaggcttaacacatgcaagtcgaacggtagcaggagaaagcttgctttcttgctgacgagtggcggacgggtgagtaatgcttgggaatctggcttatggagggggataacgacgggaaactgtcgctaataccgcgtattatcggaagatgaaagtgcgggactgagaggccgcatgccataggatgagcccaagtgggattaggtagttggtggggtaaatgcctaccaagcctgcgatctctagctggtctgagaggatgaccagccacactggaactgagacacggtccagactcctacgggaggcagcagtggggaatattgcgcaatggggggaaccctgacgcagccatgccgcgtgaatgaagaaggccttcgggttgtaaagttctttcggtattgaggaaggttgatgtgttaatagcacatcaaattgacgttaaatacagaagaagcaccggctaactccgtgccagcagccgcggtaatacggagggtgcgagcgttaatcggaataactgggcgtaaagggcacgcaggcggttatttaagtgaggtgtgaaagccccgggcttaacctgggaattgcatttcagactgggtaactagagtactttagggaggggtagaattccacgtgtagcggtgaaatgcgtagagatgtggaggaataccgaaggcgaaggcagccccttgggaatgtactgacgctcatgtgcgaaagcgtggggagcaaacaggattagataccctggtagtccacgctgtaaacgctgtcgatttgggggttggggtttaactctggcacccgtagctaacgtgataaatcgaccgcctggggagtacggccgcaaggttaaaactcaaatgaattgacgggggcccgcacaagcggtggagcatgtggtttaattcgatgcaacgcgaagaaccttacctactcttgacatcctaagaagagctcagagatgagcttgtgccttcgggaacttagagacaggtgctgcatggctgtcgtcagctcgtgttgtgaaatgttgggttaagtcccgcaacgagcgcaacccttatcctttgttgccagcgacttggtcgggaactcaaaggagactgccagtgataaactggaggaaggtggggatgacgtcaagtcatcatggcccttacgagtagggctacacacgtgctacaatggcgtatacagagggaagcgaagctgcgaggtggagcgaatctcataaagtacgtctaagtccggattggagtctgcaactcgactccatgaagtcggaatcgctagtaatcgcgaatcagaatgtcgcggtgaatacgttcccgggccttgtacacaccgcccgtcacaccatgggagtgggttgtaccagaagtagatagcttaaccttttggagggcgtttaccacggtatgattcatgactggggtg

SEQ ID NO: 81 - DNA, 407 bp, Artificial Sequence
  V3V4 fragment of Bifidobacterium longum group
tggggaatattgcacaatgggcgcaagcctgatgcagcgacgccgcgtgagggatggaggccttcgggttgtaaacctcttttatcggggagcaagcgagagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaaca

SEQ ID NO: 82 - DNA, 427 bp, Artificial Sequence
  V3V4 fragment of Lactobacillus gasseri group
tagggaatcttccacaatggacgcaagtctgatggagcaacgccgcgtgagtgaagaagggtttcggctcgtaaagctctgttggtagtgaagaaagatagaggtagtaactggcctttatttgacggtaattacttagaaagtcacggctaactacgtgccagcagccgcggtaatacgtaggtggcaagcgttgtccggatttattgggcgtaaagcgagtgcaggcggttcaataagtctgatgtgaaagccttcggctcaaccggagaattgcatcagaaactgttgaacttgagtgcagaagaggagagtggaactccatgtgtagcggtggaatgcgtagatatatggaagaacaccagtggcgaaggcggctctctggtctgcaactgacgctgaggctcgaaagcatgggtagcgaaca

SEQ ID NO: 83 - DNA, 427 bp, Artificial Sequence
  V3V4 fragment of Streptococcus peroris group
tagggaatcttcggcaatgggggcaaccctgaccgagcaacgccgcgtgagtgaagaaggttttcggatcgtaaagctctgttgtaagagaagaacgagtgtgagagtggaaagttcacgctgtgacggtatcttaccagaaagggacggctaactacgtgccagcagccgcggtaatacgtaggtcccgagcgttatccggatttattgggcgtaaagcgagcgcaggcggttagataagtctgaagttaaaggctgtggcttaaccatagtacgctttggaaactgtttaacttgagtgcaagaggggagagtggaattccatgtgtagcggtgaaatgcgtagatatatggaggaacaccggtggcgaaagcggctctctggcttgtaactgacgctgaggctcgaaagcgtggggagcaaaca

SEQ ID NO: 84 - DNA, 409 bp, Artificial Sequence
  V3V4 fragment of Bifidobacterium bifidum
tggggaatattgcacaatgggcgcaagcctgatgcagcgacgccgcgtgagggatggaggccttcgggttgtaaacctcttttgtttgggagcaagccttcgggtgagtgtacctttcgaataagcgccggctaactacgtgccagcagccgcggtaatacgtagggcgcaagcgttatccggatttattgggcgtaaagggctcgtaggcggctcgtcgcgtccggtgtgaaagtccatcgcttaacggtggatctgcgccgggtacgggcgggctggagtgcggtaggggagactggaattcccggtgtaacggtggaatgtgtagatatcgggaagaacaccgatggcgaaggcaggtctctgggccgtcactgacgctgaggagcgaaagcgtggggagcgaaca

SEQ ID NO: 85 - DNA, 427 bp, Artificial Sequence
  V3V4 fragment of Enterococcus faecalis
tagggaatcttcggcaatggacgaaagtctgaccgagcaacgccgcgtgagtgaagaaggttttcggatcgtaaaactctgttgttagagaagaacaaggacgttagtaactgaacgtcccctgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaaca

SEQ ID NO: 86 - DNA, 427 bp, Artificial Sequence
  V3V4 fragment of Streptococcus pneumoniae group
tagggaatcttcggcaatggacggaagtctgaccgagcaacgccgcgtgagtgaagaaggttttcggatcgtaaagctctgttgtaagagaagaacgagtgtgagagtggaaagttcacactgtgacggtatcttaccagaaagggacggctaactacgtgccagcagccgcggtaatacgtaggtcccgagcgttgtccggatttattgggcgtaaagcgagcgcaggcggttagataagtctgaagttaaaggctgtggcttaaccatagtaggctttggaaactgtttaacttgagtgcaagaggggagagtggaattccatgtgtagcggtgaaatgcgtagatatatggaggaacaccggtggcgaaagcggctctctggcttgtaactgacgctgaggctcgaaagcgtggggagcaaaca

SEQ ID NO: 87 - DNA, 412 bp, Artificial Sequence
  V3V4 fragment of Bifidobacterium breve
tggggaatattgcacaatgggcgcaagcctgatgcagcgacgccgcgtgagggatggaggccttcgggttgtaaacctcttttgttagggagcaaggcactttgtgttgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaaca

SEQ ID NO: 88 - DNA, 407 bp, Artificial Sequence
  V3V4 fragment of Rothia mucilaginosa group
tggggaatattgcacaatgggcgcaagcctgatgcagcgacgccgcgtgagggatgacggccttcgggttgtaaacctctgttagcagggaagaagagaaattgacggtacctgcagagaaagcgccggctaactacgtgccagcagccgcggtaatacgtagggcgcgagcgttgtccggaattattgggcgtaaagagcttgtaggcggtttgtcgcgtctgctgtgaaaggccggagcttaactccggtattgcagtgggtacgggcagactagagtgcagtaggggagactggaactcctggtgtagcggtggaatgcgcagatatcaggaagaacaccgatggcgaaggcaggtctctgggctgtaactgacgctgagaagcgaaagcatggggagcgaaca

SEQ ID NO: 89 - DNA, 427 bp, Artificial Sequence
  V3V4 fragment of Streptococcus salivarius group
tagggaatcttcggcaatgggggcaaccctgaccgagcaacgccgcgtgagtgaagaaggttttcggatcgtaaagctctgttgtaagtcaagaacgagtgtgagagtggaaagttcacactgtgacggtagcttaccagaaagggacggctaactacgtgccagcagccgcggtaatacgtaggtcccgagcgttgtccggatttattgggcgtaaagcgagcgcaggcggtttgataagtctgaagttaaaggctgtggctcaaccatagttcgctttggaaactgtcaaacttgagtgcagaaggggagagtggaattccatgtgtagcggtgaaatgcgtagatatatggaggaacaccggtggcgaaagcggctctctggtctgtaactgacgctgaggctcgaaagcgtggggagcgaaca

SEQ ID NO: 90 - DNA, 402 bp, Artificial Sequence
  V3V4 fragment of Anaerostipes hadrus group
tggggaatattgcacaatgggggaaaccctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggaattactgggtgtaaagggtgcgtaggtggtatggcaagtcagaagtgaaaacccagggcttaactctgggactgcttttgaaactgtcagactggagtgcaggagaggtaagcggaattcctagtgtagcggtgaaatgcgtagatattaggaggaacatcagtggcgaaggcggcttactggactgaaactgacactgaggcacgaaagcgtggggagcaaaca

SEQ ID NO: 91 - DNA, 427 bp, Artificial Sequence
  V3V4 fragment of Enterococcus faecium group
tagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaaca

SEQ ID NO: 92 - DNA, 402 bp, Artificial Sequence
  V3V4 fragment of Eggerthella lenta
tggggaattttgcgcaatgggggaaaccctgacgcagcaacgccgcgtgcgggacgacggccttcgggttgtaaaccgctttcagcagggaagaaattcgacggtacctgcagaagaagctccggctaactacgtgccagcagccgcggtaatacgtagggagcgagcgttatccggattcattgggcgtaaagagcgcgtaggcggcctctcaagcgggatctctaatccgagggctcaacccccggccggatcccgaactgggaggctcgagttcggtagaggcaggcggaattcccggtgtagcggtggaatgcgcagatatcgggaagaacaccgatggcgaaggcagcctgctgggccgcaactgacgctgaggcgcgaaagctaggggagcgaaca

SEQ ID NO: 93 - DNA, 409 bp, Artificial Sequence; identical to SEQ ID NO: 84
  V3V4 fragment of Bifidobacterium

SEQ ID NO: 94 - DNA, 427 bp, Artificial Sequence; identical to SEQ ID NO: 85
  V3V4 fragment of Enterococcus

SEQ ID NO: 95 - DNA, 407 bp, Artificial Sequence
  V3V4 fragment of Rothia
tggggaatattgcacaatgggcgcaagcctgatgcagcgacgccgcgtgagggatgacggccttcgggttgtaaacctctgttagcatcgaagaagcgaaagtgacggtaggtgcagagaaagcgccggctaactacgtgccagcagccgcggtaatacgtagggcgcgagcgttgtccggaattattgggcgtaaagagcttgtaggcggttggtcgcgtctgctgtgaaaggctggggcttaaccctggttttgcagtgggtacgggctaactagagtgcagtaggggagactggaattcctggtgtagcggtggaatgcgcagatatcaggaggaacaccgatggcgaaggcaggtctctgggctgtaactgacgctgagaagcgaaagcatggggagcgaaca

SEQ ID NO: 96 - DNA, 402 bp, Artificial Sequence; identical to SEQ ID NO: 92
  V3V4 fragment of Eggerthella

SEQ ID NO: 97 - DNA, 427 bp, Artificial Sequence
  V3V4 fragment of Lactobacillus
tagggaatcttccacaatggacgcaagtctgatggagcaacgccgcgtgagtgaagaaggttttcggatcgtaaagctctgttgttggtgaagaaggataggggcagtaactggtctttatttgacggtaatcaaccagaaagtcacggctaactacgtgccagcagccgcggtaatacgtaggtggcaagcgttgtccggatttattgggcgtaaagcgagcgcaggcggaatgataagtctgatgtgaaagcccacggctcaaccgtggaactgcatcggaaactgtcattcttgagtgcagaagaggagagtggaactccatgtgtagcggtggaatgcgtagatatatggaagaacaccagtggcgaaggcggctctctggtctgcaactgacgctgaggctcgaaagcatgggtagcgaaca

SEQ ID NO: 98 - DNA, 403 bp, Artificial Sequence
  V3V4 fragment of Anaerostipes
tggggaatattgcacaatgggggaaaccctgatgcagcgacgccgcgtgagtgaagaagtatttcggtatgtaaagctctatcagcagggaagaaaacagacggtacctgactaagaagccccggctaactacgtgccagcagccgcggtaatacgtagggggcaagcgttatccggaattactgggtgtaaagggtgcgtaggtggcatggtaagtcagaagtgaaagcccggggcttaaccccgggactgcttttgaaactgtcatgctggagtgcaggagaggtaagcggaattcctagtgtagcggtgaaatgcgtagatattaggaggaacaccagtggcgaaggcggcttactggactgtcactgacactgatgcacgaaagcgtggggagcaaaca

SEQ ID NO: 99 - DNA, 402 bp, Artificial Sequence
  V3V4 fragment of Fusicatenibacter saccharivorans
tggggaatattgcacaatgggggaaaccctgatgcagcgacgccgcgtgagcgaagaagtatttcggtatgtaaagctctatcagcagggaagataatgacggtacctgactaagaagccccggctaactacgtgccagcagccgcggtaatacgtagggggcaagcgttatccggatttactgggtgtaaagggagcgtagacggcaaggcaagtctgatgtgaaaacccagggcttaaccctgggactgcattggaaactgtctggctcgagtgccggagaggtaagcggaattcctagtgtagcggtgaaatgcgtagatattaggaagaacaccagtggcgaaggcggcttactggacggtaactgacgttgaggctcgaaagcgtggggagcaaaca

SEQ ID NO: 100 - DNA, 402 bp, Artificial Sequence
  V3V4 fragment of Faecalibacterium prausnitzii group
tggggaatattgcacaatgggggaaaccctgatgcagcgacgccgcgtggaggaagaaggtcttcggattgtaaactcctgttgttgaggaagataatgacggtactcaacaaggaagtgacggctaactacgtgccagcagccgcggtaaaacgtaggtcacaagcgttgtccggaattactgggtgtaaagggagcgcaggcgggaaggcaagttggaagtgaaatccatgggctcaacccatgaactgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagtgtgggtagcaaaca

SEQ ID NO: 101 - DNA, 402 bp, Artificial Sequence
  V3V4 fragment of Blautia faecis
tggggaatattgcacaatgggggaaaccctgatgcagcgacgccgcgtgaaggaagaagtatctcggtatgtaaacttctatcagcagggaagataatgacggtacctgactaagaagccccggctaactacgtgccagcagccgcggtaatacgtagggggcaagcgttatccggatttactgggtgtaaagggagcgtagacggcgcagcaagtctgatgtgaaaggcaggggcttaacccctggactgcattggaaactgctgtgcttgagtgccggaggggtaagcggaattcctagtgtagcggtgaaatgcgtagatattaggaggaacaccagtggcgaaggcggcttactggacggtaactgacgttgaggctcgaaagcgtggggagcaaaca

SEQ ID NO: 102 - DNA, 409 bp, Artificial Sequence
  V3V4 fragment of Bifidobacterium catenulatum group
tggggaatattgcacaatgggcgcaagcctgatgcagcgacgccgcgtgcgggatgacggccttcgggttgtaaaccgcttttgatcgggagcaagccttcgggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatctgcgccgggtacgggcgggctggagtgcggtaggggagactggaattcccggtgtaacggtggaatgtgtagatatcgggaagaacaccaatggcgaaggcaggtctctgggccgttactgacgctgaggagcgaaagcgtggggagcgaaca

SEQ ID NO: 103 - DNA, 402 bp, Artificial Sequence
  V3V4 fragment of Gemmiger formicilis group
tgggggatattgcacaatgggggaaaccctgatgcagcgacgccgcgtggaggaagaaggttttcggattgtaaactcctgtcgttagggacgataatgacggtacctaacaagaaagcaccggctaactacgtgccagcagccgcggtaaaacgtagggtgcaagcgttgtccggaattactgggtgtaaagggagcgcaggcggaccggcaagttggaagtgaaaactatgggctcaacccataaattgctttcaaaactgctggccttgagtagtgcagaggtaggtggaattcccggtgtagcggtggaatgcgtagatatcgggaggaacaccagtggcgaaggcgacctactgggcaccaactgacgctgaggctcgaaagcatgggtagcaaaca

SEQ ID NO: 104 - DNA, 402 bp, Artificial Sequence
  V3V4 fragment of Eubacterium eligens group
tggggaatattgcacaatggaggaaactctgatgcagcgacgccgcgtgagtgaagaagtaattcgttatgtaaagctctatcagcagggaagatagtgacggtacctgactaagaagctccggctaaatacgtgccagcagccgcggtaatacgtatggagcaagcgttatccggatttactgggtgtaaagggagtgtaggtggccatgcaagtcagaagtgaaaatccggggctcaaccccggaactgcttttgaaactgtaaggctggagtgcaggaggggtgagtggaattcctagtgtagcggtgaaatgcgtagatattaggaggaacaccagtggcgaaggcggctcactggactgtaactgacactgaggctcgaaagcgtggggagcaaaca

SEQ ID NO: 105 - DNA, 402 bp, Artificial Sequence
  V3V4 fragment of Blautia wexlerae
tggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggtgtggcaagtctgatgtgaaaggcatgggctcaacctgtggactgcattggaaactgtcatacttgagtgccggaggggtaagcggaattcctagtgtagcggtgaaatgcgtagatattaggaggaacaccagtggcgaaggcggcttactggacggtaactgacgttgaggctcgaaagcgtggggagcaaaca

SEQ ID NO: 106 - DNA, 403 bp, Artificial Sequence
  V3V4 fragment of Ruminococcus bromii
tgggggatattgcgcaatgggggcaaccctgacgcagcaacgccgcgtgaaggatgaaggttttcggattgtaaacttcttttattaaggacgaaaaatgacggtacttaatgaataagctccggctaactacgtgccagcagccgcggtaatacgtagggagcaagcgttgtccggatttactgggtgtaaagggtgcgtaggcggctttgcaagtcagatgtgaaatctatgggctcaacccataaactgcatttgaaactgtagagcttgagtgaagtagaggcaggcggaattccccgtgtagcggtgaaatgcgtagagatggggaggaacaccagtggcgaaggcggcctgctgggctttaactgacgctgaggcacgaaagcgtgggtagcaaaca

SEQ ID NO: 107 - DNA, 402 bp, Artificial Sequence
  V3V4 fragment of Eubacterium hallii
tggggaatattgcacaatgggggaaaccctgatgcagcaacgccgcgtgagtgaagaagtatttcggtatgtaaagctctatcagcagggaagataatgacggtacctgactaagaagctccggctaaatacgtgccagcagccgcggtaatacgtatggagcaagcgttatccggatttactgggtgtaaagggtgcgtaggtggcagtgcaagtcagatgtgaaaggccggggctcaaccccggagctgcatttgaaactgctcggctagagtacaggagaggcaggcggaattcctagtgtagcggtgaaatgcgtagatattaggaggaacaccagtggcgaaggcggcctgctggactgttactgacactgaggcacgaaagcgtggggagcaaaca

SEQ ID NO: 108 - DNA, 404 bp, Artificial Sequence
  V3V4 fragment of Roseburia inulinivorans
tggggaatattgcacaatgggggaaaccctgatgcagcgacgccgcgtgagcgaagaagtatttcggtatgtaaagctctatcagcagggaagaagaaatgacggtacctgactaagaagcaccggctaaatacgtgccagcagccgcggtaatacgtatggtgcaagcgttatccggatttactgggtgtaaagggagcgcaggcggaaggctaagtctgatgtgaaagcccggggctcaaccccggtactgcattggaaactggtcatctagagtgtcggaggggtaagtggaattcctagtgtagcggtgaaatgcgtagatattaggaggaacaccagtggcgaaggcggcttactggacgataactgacgctgaggctcgaaagcgtggggagcaaaca

SEQ ID NO: 109 - DNA, 402 bp, Artificial Sequence
  V3V4 fragment of LT907848_s
tggggaatattgcacaatgggggaaaccctgatgcagcaacgccgcgtgagtgaagaagtatttcggtatgtaaagctctatcagcagggaagataatgacggtacctgactaagaagctccggctaaatacgtgccagcagccgcggtaatacgtatggagcaagcgttatccggatttactgggtgtaaagggtgcgtaggtggcagtgcaagtcagatgtgaaaggccggggctcaaccccggagctgcatttgaaactgcatagctagagtacaggagaggcaggcggaattcctagtgtagcggtgaaatgcgtagatattaggaggaacaccagtggcgaaggcggcctgctggactgttactgacactgaggcacgaaagcgtggggagcaaaca

SEQ ID NO: 110 - DNA, 403 bp, Artificial Sequence
  V3V4 fragment of Roseburia cecicola group
  misc_feature (14)..(15): n is a, g, c or t
  misc_feature (31): n is a, g, c or t
  misc_feature (230): n is a, g, c or t
  misc_feature (248): n is a, g, c or t
  misc_feature (287): n is a, g, c or t
  misc_feature (294): n is a, g, c or t
tggggaatattgcnnaatgggggaaaccctnatgcagcgacgccgcgtgagcgaagaagtatttcggtatgtaaagctctatcagcagggaagaaaaatgacggtacctgactaagaagcaccggctaaatacgtgccagcagccgcggtaatacgtatggtgcmagcgttatycggatttactgggtgtmaagggagcgcmggcggtgcggcaagtctgatgtgaaagnccggggctymaccccggnactgcattggaaactgtcgtactagagtgtyggaggggnaagtggnattcctagtgtagcggtgaaatgcgtagatattaggaggaacaccagtggcgaaggcggcttactggacgattactgacgctgaggctcgaaagcgtggggagcaaaca

SEQ ID NO: 111 - DNA, 402 bp, Artificial Sequence
  V3V4 fragment of Clostridium celatum group
tggggaatattgcacaatgggggaaaccctgatgcagcaacgccgcgtgagtgatgacggccttcgggttgtaaagctctgtcttcagggacgataatgacggtacctgaggaggaagccacggctaactacgtgccagcagccgcggtaatacgtaggtggcgagcgttgtccggatttactgggcgtaaagggagcgtaggcggacttttaagtgagatgtgaaatacccgggctcaacttgggtgctgcatttcaaactggaagtctagagtgcaggagaggagaatggaattcctagtgtagcggtgaaatgcgtagagattaggaagaacaccagtggcgaaggcgattctctggactgtaactgacgctgaggctcgaaagcgtggggagcaaaca

SEQ ID NO: 112 - DNA, 402 bp, Artificial Sequence
  V3V4 fragment of PAC001046_s
tggggaatattgcacaatgggggaaaccctgatgcagcaacgccgcgtgagtgaagaagtatttcggtatgtaaagctctatcagcaggaaagaaaatgacggtacctgactaagaagccccggctaactacgtgccagcagccgcggtaatacgtagggggcaagcgttatccggatttactgggtgtaaagggagcgtagacggttttgcaagtctgaagtgaaagcccggggcttaaccccgggactgctttggaaactgtaggactagagtgcaggagaggtaagtggaattcctagtgtagcggtgaaatgcgtagatattaggaggaacaccagtggcgaaggcggcttactggactgtaactgacgttgaggctcgaaagcgtggggagcaaaca

SEQ ID NO: 113 - DNA, 402 bp, Artificial Sequence
  V3V4 fragment of Lactobacillus rogosae group
tggggaatattgcacaatggaggaaactctgatgcagcgacgccgcgtgagtgaagaagtagttcgctatgtaaagctctatcagcagggaagatagtgacggtacctgactaagaagctccggctaaatacgtgccagcagccgcggtaatacgtatggagcaagcgttatccggatttactgggtgtaaagggagtgtaggtggccaggcaagtcagaagtgaaagcccggggctcaaccccgggactgcttttgaaactgcagggctagagtgcaggaggggcaagtggaattcctagtgtagcggtgaaatgcgtagatattaggaggaacaccagtggcgaaggcggcttgctggactgtaactgacactgaggctcgaaagcgtggggagcaaaca

SEQ ID NO: 114 - DNA, 422 bp, Artificial Sequence
  V3V4 fragment of Bacteroides uniformis
tgaggaatattggtcaatggacgagagtctgaaccagccaagtagcgtgaaggatgactgccctatgggttgtaaacttcttttatacgggaataaagtgaggcacgtgtgcctttttgtatgtaccgtatgaataaggatcggctaactccgtgccagcagccgcggtaatacggaggatccgagcgttatccggatttattgggtttaaagggagcgtaggcggacgcttaagtcagttgtgaaagtttgcggctcaaccgtaaaattgcagttgatactgggtgtcttgagtacagtagaggcaggcggaattcgtggtgtagcggtgaaatgcttagatatcacgaagaactccgattgcgaaggcagcttgctggactgtaactgacgctgatgctcgaaagtgtgggtatcaaaca

SEQ ID NO: 115 - DNA, 403 bp, Artificial Sequence; identical to SEQ ID NO: 106
  V3V4 fragment of Ruminococcus_g2

SEQ ID NO: 116 - DNA, 402 bp, Artificial Sequence
  V3V4 fragment of Lachnospira
tggggaatattgcacaatggaggaaactctgatgcagcgacgccgcgtgagcgaagaagtatttcggtatgtaaagctctatcagcagggaagataatgacggtacctgactaagaagctccggctaaatacgtgccagcagccgcggtaatacgtatggagcaagcgttatccggatttactgggtgtaaagggagtgtaggtggcaaagcaagtcagtagtgaaaatccggggctcaacctcggaactgctattgaaactgtttagctagagtgcaggagaggtaagtggaattcctagtgtagcggtgaaatgcgtagatattaggaggaacaccagtggcgaaggcggcttactggactgtaactgacactgaggctcgaaagcgtggggagcaaaca

SEQ ID NO: 117 - DNA, 422 bp, Artificial Sequence
  V3V4 fragment of Bacteroides
tgaggaatattggtcaatgggcgctagcctgaaccagccaagtagcgtgaaggatgaaggctctatgggtcgtaaacttcttttatataagaataaagtgcagtatgtatactgttttgtatgtattatatgaataaggatcggctaactccgtgccagcagccgcggtaatacggaggatccgagcgttatccggatttattgggtttaaagggagcgtaggtggactggtaagtcagttgtgaaagtttgcggctcaaccgtaaaattgcagttgatactgtcagtcttgagtacagtagaggtgggcggaattcgtggtgtagcggtgaaatgcttagatatcacgaagaactccgattgcgaaggcagctcactggactgcaactgacactgatgctcgaaagtgtgggtatcaaaca

SEQ ID NO: 118 - DNA, 402 bp, Artificial Sequence; identical to SEQ ID NO: 100
  V3V4 fragment of Faecalibacterium

SEQ ID NO: 119 - DNA, 402 bp, Artificial Sequence; identical to SEQ ID NO: 107
  V3V4 fragment of Eubacterium_g5

SEQ ID NO: 120 - DNA, 402 bp, Artificial Sequence; identical to SEQ ID NO: 99
  V3V4 fragment of Fusicatenibacter

SEQ ID NO: 121 - DNA, 403 bp, Artificial Sequence; identical to SEQ ID NO: 110
  V3V4 fragment of Roseburia
  misc_feature (14)..(15): n is a, g, c or t
  misc_feature (31): n is a, g, c or t
  misc_feature (230): n is a, g, c or t
  misc_feature (248): n is a, g, c or t
  misc_feature (287): n is a, g, c or t
  misc_feature (294): n is a, g, c or t

SEQ ID NO: 122 - DNA, 403 bp, Artificial Sequence
  V3V4 fragment of Subdoligranulum
tgggggatattgcacaatgggggaaaccctgatgcagcgacgccgcgtggaggaagaaggttttcggattgtaaactcctgtcgttagggacgaatcttgacggtacctaacaagaaagcaccggctaactacgtgccagcagccgcggtaaaacgtagggtgcaagcgttgtccggaattactgggtgtaaagggagcgcaggcggaccggcaagttggaagtgaaatctatgggctcaacccataaattgctttcaaaactgctggccttgagtagtgcagaggtaggtggaattcccggtgtagcggtggaatgcgtagatatcgggaggaacaccagtggcgaaggcgacctactgggcaccaactgacgctgaggctcgaaagcatgggtagcaaaca

SEQ ID NO: 123 - DNA, 402 bp, Artificial Sequence
  V3V4 fragment of Blautia
  misc_feature (113): n is a, g, c or t
  misc_feature (162)..(164): n is a, g, c or t
  misc_feature (213): n is a, g, c or t
  misc_feature (235): n is a, g, c or t
  misc_feature (305): n is a, g, c or t
  misc_feature (383): n is a, g, c or t
tggggaatattgcacaatgggggaaaccctgatgcagcgacgccgcgtgaaggaagaagtatctcggtatgtaaacttctatcagcagggaagaaaatgacggtacctgactnagaagccccggctaactacgtgccagcagccgcggtaatacgtaggggnnnagcgttatccggatttactgggtgtaaagggagcgtagacggaagagcnagtctgatgtgaaaggctgggncttaaccccaggactgcattggaaactgttgttcgagagtgccggagaggtaagcggaattcctagtgtngcggtgaaatgcgtagatattaggaggaacaccagtggcgaaggcggcttactggacggtaactgacgttgaggctcnaaagcgtggggagcaaaca

SEQ ID NO: 124 - DNA, 426 bp, Artificial Sequence
  V3V4 fragment of CCMM_g
tagggaattttcggcaatgggggaaaccctgaccgagcaacgccgcgtgaaggaagaagtaattcgttatgtaaacttctgtcatagaggaagaacggtggatatagggaatgatatccaagtgacggtactctataagaaagccacggctaactacgtgccagcagccgcggtaatacgtaggtggcgagcgttatccggaattattgggcgtaaagagggagcaggcggcactaagggtctgtggtgaaagatcgaagcttaacttcggtaagccatggaaaccgtagagctagagtgtgtgagaggatcgtggaattccatgtgtagcggtgaaatgcgtagatatatggaggaacaccagtggcgaaggcgacgatctggcgcataactgacgctcagtcccgaaagcgtggggagcaaata

SEQ ID NO: 125 - DNA, 402 bp, Artificial Sequence
  V3V4 fragment of Agathobacter
tggggaatattgcacaatgggcgaaagcctgatgcagcgacgccgcgtgagcgaagaagtatttcggtatgtaaagctctatcagcagggaagataatgacggtacctgactaagaagcaccggctaaatacgtgccagcagccgcggtaatacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaaca

SEQ ID NO: 126 - DNA, 427 bp, Artificial Sequence
  V3V4 fragment of Parasutterella
tggggaattttggacaatgggcgcaagcctgatccagctattccgcgtgtgggatgaaggccctcgggttgtaaaccacttttgtagagaacgaaaagacaccttcgaataaagggtgttgctgacggtactctaagaataagcaccggctaactacgtgccagcagccgcggtaatacgtagggtgcgagcgttaatcggaattactgggcgtaaagggtgcgcaggcggttgagtaagacagatgtgaaatccccgagcttaactcgggaatggcatatgtgactgctcgactagagtgtgtcagagggaggtggaattccacgtgtagcagtgaaatgcgtagatatgtggaagaacaccgatggcgaaggcagcctcctgggacataactgacgctcaggcacgaaagcgtggggagcaaaca

SEQ ID NO: 127 - DNA, 403 bp, Artificial Sequence
  V3V4 fragment of Romboutsia
tggggaatattgcacaatgggcgaaagcctgatgcagcaacgccgcgtgagcgatgaaggccttcgggtcgtaaagctctgtcctcaaggaagataatgacggtacttgaggaggaagccccggctaactacgtgccagcagccgcggtaatacgtagggggctagcgttattccgaaattactgggcgaaaagggtgcgtagggtggtttctaaagtcagaggtgaaaggctacggctcaaccgtagtaagcctttgaaactggggaacttgagtgcaggagaggagagtggaattcctagtgtagcggtgaaatgcgtagatattaggaggaacaccagttgcgaaggcggctctctggactgtaactgacactgaggcacgaaagcgtggggagcaaaca

SEQ ID NO: 128 - DNA, 402 bp, Artificial Sequence; identical to SEQ ID NO: 112
  V3V4 fragment of PAC001046_g

SEQ ID NO: 129 - DNA, 403 bp, Artificial Sequence
  V3V4 fragment of Eubacterium_g23
tggggaatattgcacaatgggcgcaagcctgatgcagcaacgccgcgtggaggaagacggttttcggattgtaaactcctgttcttagtgaagaaaaatgacggtagctaaggagcaagccacggctaactacgtgccagcagccgcggtaatacgtaggtggcaagcgttgtccggaattactgggtgtaaagggagcgcaggcgggggagcaagtcagctgtgaaatctatgggcttaacccataaactgcagttgaaactgttcttcttgagtgaagtagaggttggcggaattccgagtgtagcggtgaaatgcgtagatattcggaggaacaccggtggcgaaggcggccaactgggcttttactgacgctgaggctcgaaagtgtggggagcaaaca

SEQ ID NO: 130 - DNA, 427 bp, Artificial Sequence
  V3V4 fragment of FWNZ_s
tggggaatattgcacaatgggcgcaagcctgatgcagccatgccgcgtgtgtgaagaaggccttcgggttgtaaagcactttcagcggggaggaaggcggtgaggttaataacctcatcgattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaaca

SEQ ID NO: 131 - DNA, 405 bp, Artificial Sequence
  V3V4 fragment of Flavonifractor plautii
tggggaatattgggcaatgggcgcaagcctgacccagcaacgccgcgtgaaggaagaaggctttcgggttgtaaacttcttttgtcggggacgaaacaaatgacggtacccgacgaataagccacggctaactacgtgccagcagccgcggtaatacgtaggtggcaagcgttatccggatttactgggtgtaaagggcgtgtaggcgggattgcaagtcagatgtgaaaactgggggctcaacctccagcctgcatttgaaactgtagttcttgagtgctggagaggcaatcggaattccgtgtgtagcggtgaaatgcgtagatatacggaggaacaccagtggcgaaggcggattgctggacagtaactgacgctgaggcgcgaaagcgtggggagcaaaca

SEQ ID NO: 132 - DNA, 427 bp, Artificial Sequence
  V3V4 fragment of Streptococcus gallolyticus group
tagggaatcttcggcaatgggggcaaccctgaccgagcaacgccgcgtgagtgaagaaggttttcggatcgtaaagctctgttgtaagagaagaacgtgtgtgagagtggaaagttcacacagtgacggtaacttaccagaaagggacggctaactacgtgccagcagccgcggtaatacgtaggtcccgagcgttgtccggatttattgggcgtaaagcgagcgcaggcggtttaataagtctgaagttaaaggcagtggcttaaccattgttcgctttggaaactgttaaacttgagtgcagaaggggagagtggaattccatgtgtagcggtgaaatgcgtagatatatggaggaacaccggtggcgaaagcggctctctggtctgtaactgacgctgaggctcgaaagcgtggggagcaaaca

SEQ ID NO: 133 - DNA, 402 bp, Artificial Sequence
  V3V4 fragment of Clostridium neonatale
tggggaatattgcacaatgggcgaaagcctgatgcagcaacgccgcgtgagtgatgacggccttcgggttgtaaaactctgtcttcagggacgataatgacggtacctgaggaggaagccacggctaactacgtgccagcagccgcggtaatacgtaggtggcaagcgttgtccggatttactgggcgtaaagggagcgtaggcggatgtttaagtgggatgtgaaatactcgggctcaacttgagtgctgcattccaaactggatatctagagtgcaggagaggaaaggagaattcctagtgtagcggtgaaatgcgtagagattaggaagaataccagtggcgaaggcgcctttctggactgtaactgacgctgaggctcgaaagcgtggggagcaaaca

SEQ ID NO: 134 - DNA, 401 bp, Artificial Sequence
  V3V4 fragment of Clostridioides difficile group
tggggaatattgcacaatgggcgaaagcctgatgcagcaacgccgcgtgagtgatgaaggccttcgggtcgtaaaactctgtcctcaaggaagataatgacggtacttgaggaggaagccccggctaactacgtgccagcagccgcggtaatacgtagggggctagcgttatccggatttactgggcgtaaagggtgcgtaggcggtctttcaagtcaggagtgaaaggctacggctcaaccgtagtaagctcttgaaactgggagacttgagtgcaggagaggagagtggaattcctagtgtagcggtgaaatgcgtagatattaggaggaacaccagttgcgaaggcggctctctggactgtaactgacgctgaggcacgaaagcgtggggagcaaaca

SEQ ID NO: 135 - DNA, 427 bp, Artificial Sequence
  V3V4 fragment of Veillonella ratti group
tggggaatcttccgcaatggacgaaagtctgacggagcaacgccgcgtgagtgatgacggccttcgggttgtaaagctctgttaatcgggacgaatggtctttgtgtgaataatgcaaagatttgacggtaccggaatagaaagccacggctaactacgtgccagcagccgcggtaatacgtaggtggcaagcgttgtccggaattattgggcgtaaagcgcgcgcaggcggtttcataagtctgtcttaaaagtgcggggcttaaccccgtgaggggatggaaactatggaactggagtatcggagaggaaagcggaattcctagtgtagcggtgaaatgcgtagatattaggaagaacaccagtggcgaaggcggctttctggacgacaactgacgctgaggcgcgaaagccaggggagcgaacg

SEQ ID NO: 136 - DNA, 427 bp, Artificial Sequence
  V3V4 fragment of Escherichia coli group
tggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaaca

SEQ ID NO: 137 - DNA, 402 bp, Artificial Sequence
  V3V4 fragment of Clostridium paraputrificum
tggggaatattgcacaatgggggaaaccctgatgcagcaacgccgcgtgagtgatgacggccttcgggttgtaaagctctgtctttggggacgataatgacggtacccaaggaggaagccacggctaactacgtgccagcagccgcggtaatacgtaggtggcaagcgttgtccggatttactgggcgtaaagggagcgtaggcggatttttaagtgggatgtgaaatacccgggctcaacctgggtgctgcattccaaactggaaatctagagtgcaggaggggaaagtggaattcctagtgtagcggtgaaatgcgtagagattaggaagaacaccagtggcgaaggcgactttctggactgtaactgacgctgaggctcgaaagcgtggggagcaaaca

SEQ ID NO: 138 - DNA, 422 bp, Artificial Sequence
  V3V4 fragment of Bacteroides vulgatus
tgaggaatattggtcaatgggcgagagcctgaaccagccaagtagcgtgaaggatgactgccctatgggttgtaaacttcttttataaaggaataaagtcgggtatgcatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaggaactccgattgcgaaggcagcctgctaagctgcaactgacattgaggctcgaaagtgtgggtatcaaaca

SEQ ID NO: 139 - DNA, 427 bp, Artificial Sequence
  V3V4 fragment Veillonella atypica
tggggaatcttccgcaatggacgaaagtctgacggagcaacgccgcgtgagtgatgacggccttcgggttgtaaagctctgttaatcgggacgaatggttcttgtgcgaatagtgcgaggatttgacggtaccggaatagaaagccacggctaactacgtgccagcagccgcggtaatacgtaggtggcaagcgttgtccggaattattgggcgtaaagcgcgcgcaggcggatcagttagtctgtcttaaaagttcggggcttaaccccgtgatgggatggaaactgctgatctagagtatcggagaggaaagtggaattcctagtgtagcggtgaaatgcgtagatattaggaagaacaccagtggcgaaggcgactttctggacgaaaactgacgctgaggcgcgaaagccaggggagcgaacg

SEQ ID NO: 140 - DNA, 427 bp, Artificial Sequence
  V3V4 fragment of Veillonella dispar
tggggaatcttccgcaatggacgaaagtctgacggagcaacgccgcgtgagtgatgacggccttcgggttgtaaagctctgttaatcgggacgaaaggccttcttgcgaatagttagaaggattgacggtaccggaatagaaagccacggctaactacgtgccagcagccgcggtaatacgtaggtggcaagcgttgtccggaattattgggcgtaaagcgcgcgcaggcggattggtcagtctgtcttaaaagttcggggcttaaccccgtgatgggatggaaactgccaatctagagtatcggagaggaaagtggaattcctagtgtagcggtgaaatgcgtagatattaggaagaacaccagtggcgaaggcgactttctggacgaaaactgacgctgaggcgcgaaagccaggggagcgaacg

SEQ ID NO: 141 - DNA, 405 bp, Artificial Sequence
  V3V4 fragment of Pseudoflavonifractor
tggggaatattgggcaatgggcgcaagcctgacccagcaacgccgcgtgaaggatgaaggctttcgggttgtaaacttcttttatcagggacgaaataaatgacggtacctgatgaataagccacggctaactacgtgccagcagccgcggtaatacgtaggtggcaagcgttatccggatttactgggtgtaaagggcgtgtaggcgggactgcaagtcaggtgtgaaaaccacgggctcaacctgtggcctgcatttgaaactgtagttcttgagtgctggagaggcaatcggaattccgtgtgtagcggtgaaatgcgtagatatacggaggaacaccagtggcgaaggcggattgctggacagtaactgacgctgaggcgcgaaagcgtggggagcaaaca

SEQ ID NO: 142 - DNA, 401 bp, Artificial Sequence; identical to SEQ ID NO: 134
  V3V4 fragment of Clostridioides

SEQ ID NO: 143 - DNA, 427 bp, Artificial Sequence; identical to SEQ ID NO: 136
  V3V4 fragment of Escherichia

SEQ ID NO: 144 - DNA, 402 bp, Artificial Sequence
  V3V4 fragment of Clostridium_g24
  misc_feature (164): n is a, g, c or t
tggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcnagcgttatccggatttactgggtgtaaagggagcgtagacggcgaagcaagtctgaagtgaaaacccagggctcaaccctggcactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaaca

SEQ ID NO: 145 - DNA, 402 bp, Artificial Sequence
  V3V4 fragment of Clostridium
tggggaatattgcacaatgggggaaaccctgatgcagcaacgccgcgtgagtgatgacggtcttcggattgtaaagctctgtctttagggacgataatgacggtacctaaggaggaagccacggctaactacgtgccagcagccgcggtaatacgtaggtggcaagcgttgtccggatttactgggcgtaaagggagcgtaggtggatatttaagtgggatgtgaaatacccgggcttaacctgggtgctgcattccaaactggatatctagagtgcaggagaggaaaggagaattcctagtgtagcggtgaaatgcgtagagattaggaagaataccagtggcgaaggcgactttctggactgtaactgacactgaggctcgaaagcgtggggagcaaaca

SEQ ID NO: 146 - DNA, 427 bp, Artificial Sequence
  V3V4 fragment of Veillonella
tggggaatcttccgcaatggacgaaagtctgacggagcaacgccgcgtgagtgatgacggccttcgggttgtaaagctctgttaatcgggacgaaaggccttcttgcgaacagttagaaggattgacggtaccggaatagaaagccacggctaactacgtgccagcagccgcggtaatacgtaggtggcaagcgttgtccggaattattgggcgtaaagcgcgcgcaggcggatcagtcagtctgtcttaaaagttcggggcttaaccccgtgatgggatggaaactgctgatctagagtatcggagaggaaagtggaattcctagtgtagcggtgaaatgcgtagatattaggaagaacaccagtggcgaaggcgactttctggacgaaaactgacgctgaggcgcgaaagccaggggagcgaacg

SEQ ID NO: 147 - DNA, 422 bp, Artificial Sequence
  V3V4 fragment of Bacteroides dorei
tgaggaatattggtcaatgggcgatggcctgaaccagccaagtagcgtgaaggatgactgccctatgggttgtaaacttcttttataaaggaataaagtcgggtatgcatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatgtcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaaca

SEQ ID NO: 148 - DNA, 402 bp, Artificial Sequence
  V3V4 fragment of PAC001148_s
tggggaatattgcacaatgggggaaaccctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatagcaagtctggagtgaaagcccggggctcaaccccggtactgctttggaaactgttaagctagagtgctggagaggtaagtggaattcctagtgtagcggtgaaatgcgtagatattaggaggaacaccagtggcgaaggcggcttactggacagtaactgacgttgaggctcgaaagcgtggggagcaaaca

SEQ ID NO: 149 - DNA, 427 bp, Artificial Sequence
  V3V4 fragment of Haemophilus parainfluenzae group
tggggaatattgcgcaatgggggcaaccctgacgcagccatgccgcgtgaatgaagaaggccttcgggttgtaaagttctttcggtagcgaggaaggcatttagtttaatagactaggtgattgacgttaactacagaagaagcaccggctaactccgtgccagcagccgcggtaatacggagggtgcgagcgttaatcggaataactgggcgtaaagggcacgcaggcggtgacttaagtgaggtgtgaaagccccgggcttaacctgggaattgcatttcatactgggtcgctagagtactttagggaggggtagaattccacgtgtagcggtgaaatgcgtagagatgtggaggaataccgaaggcgaaggcagccccttgggaatgtactgacgctcatgtgcgaaagcgtggggagcaaaca

SEQ ID NO: 150 - DNA, 427 bp, Artificial Sequence
  V3V4 fragment of Lactobacillus paracasei group
tagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagcgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaaca

SEQ ID NO: 151 - DNA, 422 bp, Artificial Sequence
  V3V4 fragment of Bacteroides ovatus group
tgaggaatattggtcaatgggcgagagcctgaaccagccaagtagcgtgaaggatgaaggctctatgggtcgtaaacttcttttatatgggaataaagttttccacgtgtggaattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgttactgacactgatgctcgaaagtgtgggtatcaaaca

SEQ ID NO: 152 - DNA, 427 bp, Artificial Sequence
  V3V4 fragment of Lactobacillus fermentum
tagggaatcttccacaatgggcgcaagcctgatggagcaacaccgcgtgagtgaagaagggtttcggctcgtaaagctctgttgttaaagaagaacacgtatgagagtaactgttcatacgttgacggtatttaaccagaaagtcacggctaactacgtgccagcagccgcggtaatacgtaggtggcaagcgttatccggatttattgggcgtaaagagagtgcaggcggttttctaagtctgatgtgaaagccttcggcttaaccggagaagtgcatcggaaactggataacttgagtgcagaagagggtagtggaactccatgtgtagcggtggaatgcgtagatatatggaagaacaccagtggcgaaggcggctacctggtctgcaactgacgctgagactcgaaagcatgggtagcgaaca

SEQ ID NO: 153 - DNA, 402 bp, Artificial Sequence
  V3V4 fragment of Clostridium_g35
tggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggtaaagcaagtctgaagtgaaagcccgcggctcaactgcgggactgctttggaaactgtttaactggagtgtcggagaggtaagtggaattcctagtgtagcggtgaaatgcgtagatattaggaggaacaccagtggcgaaggcgacttactggacgataactgacgttgaggctcgaaagcgtggggagcaaaca

SEQ ID NO: 154 - DNA, 401 bp, Artificial Sequence
  V3V4 fragment of Intestinibacter
tggggaatattgcacaatgggcgaaagcctgatgcagcaacgccgcgtgagcgatgaaggccttcgggtcgtaaagctctgtcctcaaggaagataatgacggtacttgaggaggaagccccggctaactacgtgccagcagccgcggtaatacgtagggggctagcgttatccggatttactgggcgtaaagggtgcgtaggcggtcttttaagtcaggagtgaaaggctacggctcaaccgtagtaagctcttgaaactggaggacttgagtgcaggagaggagagtggaattcctagtgtagcggtgaaatgcgtagatattaggaggaacaccagtagcgaaggcggctctctggactgtaactgacgctgaggcacgaaagcgtggggagcaaaca

SEQ ID NO: 155 - DNA, 402 bp, Artificial Sequence
  V3V4 fragment of Hungatella
tggggaatattggacaatgggcgaaagcctgatccagcgacgccgcgtgagtgaagaagtgtttcggcatgtaaagctctatcagcagggaagaaaatgacggtacctgactaagaagccccggctaactacgtgccagcagccgcggtaatacgtagggggcaagcgttatccggatttactgggtgtaaagggagcgtagacggttaagcaagtctgaagtgaaagcccggggctcaaccccggtactgctttggaaactgtttgacttgagtgcaggagaggtaagtggaattcctagtgtagcggtgaaatgcgtagatattaggaggaacaccagtggcgaaggcggcttactggactgtaactgacgttgaggctcgaaagcgtggggagcaaaca

SEQ ID NO: 156 - DNA, 422 bp, Artificial Sequence
  V3V4 fragment of Prevotella
tgaggaatattggtcaatggacggaagtctgaaccagccaagtagcgtgcaggatgacggccctatgggttgtaaactgcttttgtatggggataaagttagggacgtgtccctatttgcaggtaccatacgaataaggaccggctaattccgtgccagcagccgcggtaatacggaaggtccaggcgttatccggatttattgggtttaaagggagcgtaggctggagattaagtgtgttgtgaaatgtagacgctcaacgtctgaattgcagcgcatactggtttccttgagtacgcacaacgttggcggaattcgtcgtgtagcggtgaaatgcttagatatgacgaagaactccgattgcgaaggcagctgacgggagcgcaactgacgcttaagctcgaaggtgcgggtatcaaaca

SEQ ID NO: 157 - DNA, 427 bp, Artificial Sequence
  V3V4 fragment of Streptococcus
tagggaatcttcggcaatgggggcaaccctgaccgagcaacgccgcgtgagtgaagaaggttttcggatcgtaaagctctgttgttagagaagaatgatggtgggagtggaaaatccaccaagtgacggtaactaaccagaaagggacggctaactacgtgccagcagccgcggtaatacgtaggtcccgagcgttgtccggatttattgggcgtaaagcgagcgcaggcggttttttaagtctgaagttaaaggcattggctcaaccaatgtacgctttggaaactggagaacttgagtgcagaaggggagagtggaattccatgtgtagcggtgaaatgcgtagatatatggaggaacaccggtggcgaaagcggctctctggtctgtaactgacgctgaggctcgaaagcgtggggagcaaaca

SEQ ID NO: 158 - DNA, 427 bp, Artificial Sequence
  V3V4 fragment of Citrobacter
tggggaatattgcacaatgggcgcaagcctgatgcagccatgccgcgtgtatgaagaaggccttcgggttgtaaagtactttcagcgaggaggaaggcgttgtggttaataaccgcagcgattgacgttactcgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcatccgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaaca

SEQ ID NO: 159 - DNA, 427 bp, Artificial Sequence
  V3V4 fragment of Klebsiella
tggggaatattgcacaatgggcgcaagcctgatgcagccatgccgcgtgtgtgaagaaggccttcgggttgtaaagcactttcagcggggaggaaggcgatgaggttaataacctcatcgattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaaca

SEQ ID NO: 160 - DNA, 427 bp, Artificial Sequence
  V3V4 fragment of Haemophilus
tggggaatattgcgcaatggggggaaccctgacgcagccatgccgcgtgaatgaagaaggccttcgggttgtaaagttctttcggtattgaggaaggttgatgtgttaatagcacatcaaattgacgttaaatacagaagaagcaccggctaactccgtgccagcagccgcggtaatacggagggtgcgagcgttaatcggaataactgggcgtaaagggcacgcaggcggttatttaagtgaggtgtgaaagccccgggcttaacctgggaattgcatttcagactgggtaactagagtactttagggaggggtagaattccacgtgtagcggtgaaatgcgtagagatgtggaggaataccgaaggcgaaggcagccccttgggaatgtactgacgctcatgtgcgaaagcgtggggagcaaaca

SEQ ID NO: 161 - DNA, 17 bp, Artificial Sequence
  Forward universal primer
  misc_feature (9): n is a, g, c or t
cctacgggnggcwgcag

SEQ ID NO: 162 - DNA, 21 bp, Artificial Sequence
  Reverse universal primer
gactachvgggtatctaatcc